(19) United States
(12) Patent Application Publication
(10) Pub. No.: US 2004/0197804 A1
(43) Pub. Date: Oct. 7, 2004

(54) Method for in vitro selection of 2'-substituted nucleic acids

(76) Inventors: Anthony D. Keefe, Cambridge, MA (US); Charles Wilson, Concord, MA (US); Paula Burmeister, Cambridge, MA (US); Sara Chesworth Keene, Tewksbury, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/729,581

(22) Filed: Dec. 3, 2003

Related U.S. Application Data
(60) Provisional application No. 60/430,761, filed on Dec. 3, 2002.
(60) Provisional application No. 60/487,474, filed on Jul. 15, 2003.
(60) Provisional application No. 60/517,039, filed on Nov. 4, 2003.

(51) Int. Cl. C12Q 1/68; C12P 19/34

(52) U.S. Cl. 435/6; 435/91.2

(57) ABSTRACT

Materials and methods are provided for producing aptamer therapeutics having modified nucleotide triphosphates incorporated into their sequence. The aptamers produced by the methods of the invention have increased stability and half life.

### REFERENCE TO RELATED APPLICATIONS

This application is related to and claims priority to U.S. Provisional Patent Application Serial No. 60/430,761, filed Dec. 3, 2002, U.S. Provisional Patent Application Serial No. 60/487,474, filed Jul. 15, 2003, and U.S. Provisional Patent Application Serial No. 60/517,039, filed Nov. 4, 2003, each of which is herein incorporated by reference in its entirety.

### FIELD OF THE INVENTION

The invention relates generally to the field of nucleic acids and more particularly to aptamers, and methods for selecting aptamers, incorporating modified nucleotides. The invention further relates to materials and methods for enzymatically producing pools of randomized oligonucleotides having modified nucleotides from which, e.g., aptamers to a specific target can be selected.

### BACKGROUND OF THE INVENTION

Aptamers are nucleic acid molecules having specific binding affinity to molecules through interactions other than classic Watson-Crick base pairing.

Aptamers, like peptides generated by phage display or monoclonal antibodies (MAbs), are capable of specifically binding to selected targets and, through binding, block their targets' ability to function. Created by an in vitro selection process from pools of random sequence oligonucleotides (FIG. 1), aptamers have been generated for over 100 proteins including growth factors, transcription factors, enzymes, immunoglobulins, and receptors. A typical aptamer is 10-15 kDa in size (30-45 nucleotides), binds its target with sub-nanomolar affinity, and discriminates against closely related targets (e.g., will typically not bind other proteins from the same gene family). A series of structural studies have shown that aptamers are capable of using the same types of binding interactions (hydrogen bonding, electrostatic complementarity, hydrophobic contacts, steric exclusion, etc) that drive affinity and specificity in antibody-antigen complexes.

Aptamers have a number of desirable characteristics for use as therapeutics (and diagnostics) including high specificity and affinity, biological efficacy, and excellent pharmacokinetic properties. In addition, they offer specific competitive advantages over antibodies and other protein biologics, for example:

1) Speed and control. Aptamers are produced by an entirely in vitro process, allowing for the rapid generation of initial (therapeutic) leads. In vitro selection allows the specificity and affinity of the aptarner to be tightly controlled and allows the generation of leads against both toxic and non-immunogenic targets.

2) Toxicity and Immunogenicity. Aptamers as a class have demonstrated little or no toxicity or immunogenicity. In chronic dosing of rats or woodchucks with high levels of aptamer (10 mg/kg daily for 90 days), no toxicity is observed by any clinical, cellular, or biochemical measure. Whereas the efficacy of many monoclonal antibodies can be severely limited by immune response to antibodies themselves, it is extremely difficult to elicit antibodies to aptamers (most likely because aptamers cannot be presented by T-cells via the MHC and the immune response is generally trained not to recognize nucleic acid fragments).

3) Administration. Whereas all currently approved antibody therapeutics are administered by intravenous infusion (typically over 2-4 hours), aptamers can be administered by subcutaneous injection. This difference is primarily due to the comparatively low solubility and thus large volumes necessary for most therapeutic MAbs. With good solubility (>150 mg/ml) and comparatively low molecular weight (aptarner: 10-50 kDa; antibody: 150 kDa), a weekly dose of aptarner may be delivered by injection in a volume of less than 0.5 ml. Aptamer bioavailability via subcutaneous administration is >80% in monkey studies (Tucker et al., J. Chromatography B. 732: 203-12, 1999). In addition, the small size of aptamers allows them to penetrate into areas of conformational constrictions that do not allow for antibodies or antibody fragments to penetrate, presenting yet another advantage of aptamer-based therapeutics or prophylaxis.

4) Scalability and cost. Therapeutic aptamers are chemically synthesized and consequently can be readily scaled as needed to meet production demand. Whereas difficulties in scaling production are currently limiting the availability of some biologics and the capital cost of a large-scale protein production plant is enormous, a single large-scale synthesizer can produce upwards of 100 kg oligonucleotide per year and requires a relatively modest initial investment. The current cost of goods for aptamer synthesis at the kilogram scale is estimated at $500/g, comparable to that for highly optimized antibodies. Continuing improvements in process development are expected to lower the cost of goods to <$100/g in five years.

5) Stability. Therapeutic aptamers are chemically robust. They are intrinsically adapted to regain activity following exposure to heat, denaturants, etc. and can be stored for extended periods (>1 yr) at room temperature as lyophilized powders. In contrast, antibodies must be stored refrigerated.

Given the advantages of aptamers as therapeutic agents, it would be beneficial to have materials and methods to prolong or increase the stability of aptamer therapeutics in vivo. The present invention provides materials and methods to meet these and other needs.

### BRIEF DESCRIPTION OF THE DRAWINGS

FIG. 1 is a schematic representation of the in vitro aptamer selection (SELEX™) process from pools of random sequence oligonucleotides.

FIG. 2 shows a 2′-O-methyl (2′-OMe) modified nucleotide, where “B” is a purine or pyrimidine base.

FIG. 3A is a graph of VEGF-binding by three 2′-OMe VEGF aptamers: ARC224, ARC245 and ARC259; FIG. 3B shows the sequences and putative secondary structures of these aptamers.

FIG. 4 is a graph of the VEGF-binding by various 2′-OH G variants of ARC224 and ARC225

FIG. 5 is a graph of ARC224 binding to VEGF in HUVEC.

FIG. 6 is a graph of ARC224 binding to VEGF before and after autoclaving, in the presence or absence of EDTA.

FIGS. 7A and 7B are graphs of the stability of ARC224 and ARC226, respectively, when incubated at 37° C. in rat plasma.

FIG. 8 is a graph of dRmY SELEX™ Round 6 sequences binding to IgE.

FIG. 9 is a graph of dRmY SELEX™ Round 6 sequences binding to thrombin.

FIG. 10 is a graph of dRmY SELEX™ Round 6 sequences binding to VEGF.

FIG. 11A is a degradation plot of an all 2′-OMe oligonucleotide with 3′-idT, in 95% rat plasma (citrated) at 37° C., and FIG. 11B is a degradation plot of the corresponding dRmY oligonucleotide in 95% rat plasma at 37° C.

FIG. 12 is a graph of rGmH h-IgE binding clones (Round 6).

FIG. 13A is a graph of round 12 pools for rRmY pool PDGF-BB selection, and FIG. 13B is a graph of Round 10 pools for rGmH pool PDGF-BB selection.

FIG. 14 is a graph of dRmY SELEX™ Round 6, 7, 8 and unselected sequences binding to IL-23.

FIG. 15 is a graph of dRmY SELEX™ Round 6, 7 and unselected sequences binding to PDGF-BB.

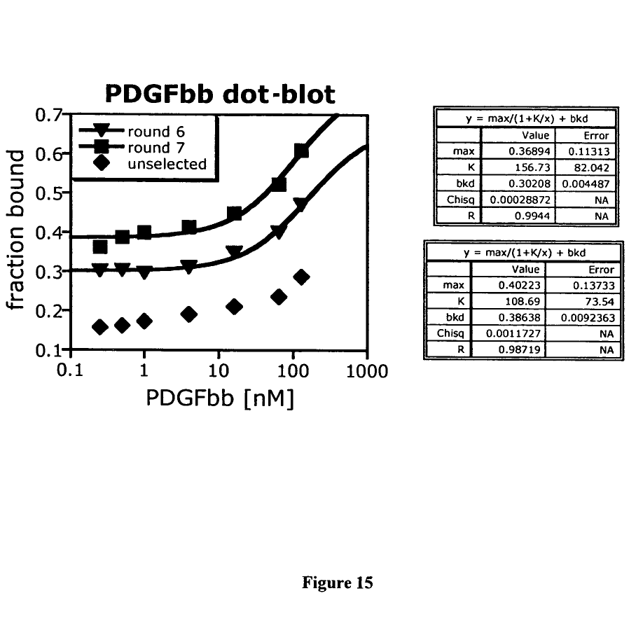

### SUMMARY OF THE INVENTION

The present invention provides materials and methods to produce oligonucleotides of increased stability by transcription under the conditions specified herein which promote the incorporation of modified nucleotides into the oligonucleotide. These modified oligonucleotides can be, for example, aptamers, antisense molecules, RNAi molecules, siRNA molecules, or ribozymes. Preferably, the oligonucleotide is an aptamer.

In one embodiment, the present invention provides an improved SELEX™ method (“2″-OMe SELEX™”) that uses randomized pools of oligonucleotides incorporating modified nucleotides from which aptamers to a specific target can be selected.

In one embodiment, the present invention provides methods that use modified enzymes to incorporate modified nucleotides into oligonucleotides under a given set of transcription conditions.

In one embodiment, the present invention provides methods that use a mutated polymerase. In one embodiment, the mutated polymerase is a T7 RNA polymerase. In one embodiment, a T7 RNA polymerase modified by having a mutation at position 639 (from a tyrosine residue to a phenylalanine residue “Y639F”) and at position 784 (from a histidine residue to an alanine residue “H784A”) is used in various transcription reaction conditions which result in the incorporation of modified nucleotides into the oligonucleotides of the invention.

In another embodiment, a T7 RNA polymerase modified with a mutation at position 639 (from a tyrosine residue to a phenylalanine residue) is used in various transcription reaction conditions which result in the incorporation of modified nucleotides into the oligonucleotides of the invention.

In another embodiment, a T7 RNA polymerase modified with a mutation at position 784 (from a histidine residue to an alanine residue) is used in various transcription reaction conditions which result in the incorporation of modified nucleotides into the aptamers of the invention.

In one embodiment, the present invention provides various transcription reaction mixtures that increase the incorporation of modified nucleotides by the modified enzymes of the invention.

In one embodiment, manganese ions are added to the transcription reaction mixture to increase the incorporation of modified nucleotides by the modified enzymes of the invention.

In another embodiment, 2′-OH GTP is added to the transcription mixture to increase the incorporation of modified nucleotides by the modified enzymes of the invention.

In another embodiment, polyethylene glycol, PEG, is added to the transcription mixture to increase the incorporation of modified nucleotides by the modified enzymes of the invention.

In another embodiment, GMP (or any substituted guanosine) is added to the transcription mixture to increase the incorporation of modified nucleotides by the modified enzymes of the invention.

In one embodiment, a leader sequence incorporated into the 5′ end of the fixed region (preferably 20-25 nucleotides in length) at the 5′ end of a template oligonucleotide is used to increase the incorporation of modified nucleotides by the modified enzymes of the invention. Preferably, the leader sequence is greater than about 10 nucleotides in length.

In one embodiment, a leader sequence that is composed of up to 100% (inclusive) purine nucleotides is used.

In another embodiment, a leader sequence at least 6 nucleotides long that is composed of up to 100% (inclusive) purine nucleotides is used.

In another embodiment, a leader sequence at least 8 nucleotides long that is composed of up to 100% (inclusive) purine nucleotides is used.

In another embodiment, a leader sequence at least 10 nucleotides long that is composed of up to 100% (inclusive) purine nucleotides is used.

In another embodiment, a leader sequence at least 12 nucleotides long that is composed of up to 100% (inclusive) purine nucleotides is used.

In another embodiment, a leader sequence at least 14 nucleotides long that is composed of up to 100% (inclusive) purine nucleotides is used.

In one embodiment, the present invention provides aptamer therapeutics having modified nucleotides incorporated into their sequence.

In one embodiment, the present invention provides for the use of aptamer therapeutics having modified nucleotides incorporated into their sequence.

In one embodiment, the present invention provides various compositions of nucleotides for transcription for the selection of aptamers with the SELEX™ process. In one embodiment, the present invention provides combinations of 2′-OH, 2′-F, 2′-deoxy, and 2′-OMe modifications of the ATP, GTP, CTP, TTP, and UTP nucleotides. In another embodiment, the present invention provides combinations of 2′-OH, 2′-F, 2′-deoxy, 2′-OMe, 2′-NH₂, and 2′-methoxyethyl modifications of the ATP, GTP, CTP, TTP, and UTP nucleotides. In one embodiment, the present invention provides 5 combinations of 2′-OH, 2′-F, 2′-deoxy, 2′-OMe, 2′-NH₂, and 2′-methoxyethyl modifications the ATP, GTP, CTP, TTP, and UTP nucleotides.

The invention relates to a method for identifying nucleic acid ligands to a target molecule, where the ligands include modified nucleotides, by: a) preparing a transcription reaction mixture comprising a mutated polymerase, one or more 2′-modified nucleotide triphosphates (NTPs), magnesium ions and one or more oligonucleotide transcription templates; b) preparing a candidate mixture of single-stranded nucleic acids by transcribing the one or more oligonucleotide transcription templates under conditions whereby the mutated polymerase incorporates at least one of the one or more modified nucleotides into each nucleic acid of the candidate mixture, wherein each nucleic acid of the candidate mixture comprises a 2′-modified nucleotide selected from the group consisting of a 2′-position modified pyrimidine and a 2′-position modified purine; c) contacting the candidate mixture with the target molecule; d) partitioning the nucleic acids having an increased affinity to the target molecule relative to the candidate mixture from the remainder of the candidate mixture; and e) amplifying the increased affinity nucleic acids, in vitro, to yield a ligand-enriched mixture of nucleic acids.

The 2′-position modified pyrimidines and 2′-position modified purines include 2′-OH, 2′-deoxy, 2′-O-methyl, 2′-NH₂, 2′-F, and 2′-methoxy ethyl modifications. Preferably, the 2′-modified nucleotides are 2′-O-methyl or 2′-F nucleotides.

In some embodiments, the mutated polymerase is a mutated T7 RNA polymerase, such as a T7 RNA polymerase having a mutation at position 639 from a tyrosine residue to a phenylalanine residue (Y639F); a T7 RNA polymerase having a mutation at position 784 from a histidine residue to an alanine residue (H784A); a T7 RNA polymerase having a mutation at position 639 from a tyrosine residue to a phenylalanine residue and a mutation at position 784 from a histidine residue to an alanine residue (Y639F/H784A).

In some embodiments, the oligonucleotide transcription template includes a leader sequence incorporated into the 5′ end of a fixed region at the 5′ end of the oligonucleotide transcription template. The leader sequence, for example, is an all-purine leader sequence. The leader sequence, for example, can be at least 6 nucleotides long; at least 8 nucleotides long; at least 10 nucleotides long; at least 12 nucleotides long; or at least 14 nucleotides long.

In some embodiments, the transcription reaction mixture also includes manganese ions. For example, the concentration of magnesium ions is between 3.0 and 3.5 times greater than the concentration of manganese ions.

In some embodiments of the transcription reaction mixture, each NTP is present at a concentration of 0.5 mM, the concentration of magnesium ions is 5.0 mM, and the concentration of manganese ions is 1.5 mM. In other embodiments of the transcription reaction mixture each NTP is present at a concentration of 1.0 mM, the concentration of magnesium ions is 6.5 mM, and the concentration of manganese ions is 2.0 mM. In other embodiments of the transcription reaction mixture each NTP is present at a concentration of 2.0 mM, the concentration of magnesium ions is 9.6 mM, and the concentration of manganese ions is 2.9 mM.

In some embodiments, the transcription reaction mixture also includes 2′-OH GTP.

In some embodiments, the transcription reaction mixture also includes a polyalkylene glycol. The polyalkylene glycol can be, e.g., polyethylene glycol (PEG).

In some embodiments, the transcription reaction mixture also includes GMP.

In some embodiments, the method for identifying nucleic acid ligands to a target molecule further includes repeating steps d) partitioning the nucleic acids having an increased affinity to the target molecule relative to the candidate mixture from the remainder of the candidate mixture; and e) amplifying the increased affinity nucleic acids, in vitro, to yield a ligand-enriched mixture of nucleic acids.

In some aspects, the invention relates to a nucleic acid ligand to thrombin which was identified according to the method of the invention.

In some aspects, the invention relates to a nucleic acid ligand to vascular endothelial growth factor (VEGF) which was identified according to the method of the invention.

In some aspects, the invention relates to a nucleic acid ligand to IgE which was identified according to the method of the invention.

In some aspects, the invention relates to a nucleic acid ligand to IL-23 which was identified according to the method of the invention.

In some aspects, the invention relates to a nucleic acid ligand to platelet-derived growth factor-BB (PDGF-BB) which was identified according to the method of the invention.

In some embodiments, the transcription reaction mixture includes 2′-OH adenosine triphosphate (ATP), 2′-OH guanosine triphosphate (GTP), 2′-O-methyl cytidine triphosphate (CTP) and 2′-O-methyl uridine triphosphate (UTP).

In some embodiments, the transcription reaction mixture includes 2′-deoxy purine nucleotide triphosphates and 2′-O-methylpyrimidine nucleotide triphosphates.

In some embodiments, the transcription reaction mixture includes 2′-O-methyl adenosine triphosphate (ATP), 2′-OH guanosine triphosphate (GTP), 2′-O-methyl cytidine triphosphate (CTP) and 2′-O-methyl uridine triphosphate (UTP).

In some embodiments, the transcription reaction mixture includes 2′-O-methyl adenosine triphosphate (ATP), 2′-O-methyl cytidine triphosphate (CTP) and 2′-O-methyl uridine triphosphate (UTP), 2′-O-methyl guanosine triphosphate (GTP) and deoxy guanosine triphosphate (GTP), wherein the deoxy guanosine triphosphate comprises a maximum of 10% of the total guanosine triphosphate population.

In some embodiments, the transcription reaction mixture includes 2′-O-methyl adenosine triphosphate (ATP), 2′-F guanosine triphosphate (GTP), 2′-O-methyl cytidine triphosphate (CTP) and 2′-O-methyl uridine triphosphate (UTP).

In some embodiments, the transcription reaction mixture includes 2′-deoxy adenosine triphosphate (ATP), 2′-O-methyl guanosine triphosphate (GTP), 2′-O-methyl cytidine triphosphate (CTP) and 2′-O-methyl uridine triphosphate (UTP).

The invention also relates to a method of preparing a nucleic acid comprising one or more modified nucleotides by: preparing a transcription reaction mixture comprising a mutated polymerase, one or more 2′-modified nucleotide triphosphates (NTPs), magnesium ions and one or more oligonucleotide transcription templates; and contacting the one or more oligonucleotide transcription templates with the mutated polymerase under conditions whereby the mutated polymerase incorporates the one or more 2′-modified nucleotides into a nucleic acid transcription product.

2′-position modified pyrimidines and 2′-position modified purines include 2′-OH, 2′-deoxy, 2′-O-methyl, 2′-NH₂, 2′-F, and 2′-methoxy ethyl modifications. Preferably, the 2′-modified nucleotides are 2′-O-methyl or 2′-F nucleotides.

In some embodiments, the mutated polymerase is a mutated T7 RNA polymerase, such as a T7 RNA polymerase having a mutation at position 639 from a tyrosine residue to a phenylalanine residue (Y639F); a T7 RNA polymerase having a mutation at position 784 from a histidine residue to an alanine residue (H784A); a T7 RNA polymerase having a mutation at position 639 from a tyrosine residue to a phenylalanine residue and a mutation at position 784 from a histidine residue to an alanine residue (Y639F/H784A).

In some embodiments, the oligonucleotide transcription template includes a leader sequence incorporated into the 5′ end of a fixed region at the 5′ end of the oligonucleotide transcription template. The leader sequence, for example, is an all-purine leader sequence. The leader sequence, for example, can be at least 6 nucleotides long; at least 8 nucleotides long; at least 10 nucleotides long; at least 12 nucleotides long; or at least 14 nucleotides long.

In some embodiments, the transcription reaction mixture also includes manganese ions. For example, the concentration of magnesium ions is between 3.0 and 3.5 times greater than the concentration of manganese ions.

In some embodiments of the transcription reaction mixture, each NTP is present at a concentration of 0.5 mM, the concentration of magnesium ions is 5.0 mM, and the concentration of manganese ions is 1.5 mM. In other embodiments of the transcription reaction mixture each NTP is present at a concentration of 1.0 mM, the concentration of magnesium ions is 6.5 mM, and the concentration of manganese ions is 2.0 mM. In other embodiments of the transcription reaction mixture each NTP is present at a concentration of 2.0 mM, the concentration of magnesium ions is 9.6 mM, and the concentration of manganese ions is 2.9 mM.

In some embodiments, the transcription reaction mixture also includes 2′-OH GTP.

In some embodiments, the transcription reaction mixture also includes a polyalkylene glycol. The polyalkylene glycol can be, e.g., polyethylene glycol (PEG).

In some embodiments, the transcription reaction mixture also includes GMP.

The invention also relates to an aptamer composition comprising a sequence where substantially all adenosine nucleotides are 2′-OH adenosine, substantially all guanosine nucleotides are 2′-OH guanosine, substantially all cytidine nucleotides are 2′-O-methyl cytidine, and substantially all uridine nucleotides are 2′-O-methyl uridine. In one embodiment, the aptamer has a sequence composition where at least 80% of all adenosine nucleotides are 2′-OH adenosine, at least 80% of all guanosine nucleotides are 2′-OH guanosine, at least 80% of all cytidine nucleotides are 2′-O-methyl cytidine and at least 80% of all uridine nucleotides are 2′-O-methyl uridine. In another embodiment, the aptamer has a sequence composition where at least 90% of all adenosine nucleotides are 2′-OH adenosine, at least 90% of all guanosine nucleotides are 2′-OH guanosine, at least 90% of all cytidine nucleotides are 2′-O-methyl cytidine and at least 90% of all uridine nucleotides are 2′-O-methyl uridine. In another embodiment, the aptamer has a sequence composition where 100% of all adenosine nucleotides are 2′-OH adenosine, at 100% of all guanosine nucleotides are 2′-OH guanosine, 100% of all cytidine nucleotides are 2′-O-methyl cytidine and 100% of all uridine nucleotides are 2′-O-methyl uridine.

The invention also relates to an aptamer composition comprising a sequence where substantially all purine nucleotides are 2′-deoxy purines and substantially all pyrimidine nucleotides are 2′-O-methylpyrimidines. In one embodiment, the aptamer has a sequence composition where at least 80% of all purine nucleotides are 2′-deoxy purines and at least 80% of all pyrimidine nucleotides are 2′-O-methylpyrimidines. In another embodiment, the aptamer has a sequence composition where at least 90% of all purine nucleotides are 2′-deoxy purines and at least 90% of all pyrimidine nucleotides are 2′-O-methylpyrimidines. In another embodiment, the aptamer has a sequence composition where 100% of all purine nucleotides are 2′-deoxy purines and 100% of all pyrimidine nucleotides are 2′-O-methylpyrimidines.

The invention also relates to an aptamer composition comprising a sequence where substantially all guanosine nucleotides are 2′-OH guanosine, substantially all cytidine nucleotides are 2′-O-methyl cytidine, substantially all uridine nucleotides are 2′-O-methyl uridine, and substantially all adenosine nucleotides are 2′-O-methyl adenosine. In one embodiment, the aptamer has a sequence composition where at least 80% of all guanosine nucleotides are 2′-OH guanosine, at least 80% of all cytidine nucleotides are 2′-O-methyl cytidine, at least 80% of all uridine nucleotides are 2′-O-methyl uridine, and at least 80% of all adenosine nucleotides are 2′-O-methyl adenosine. In another embodiment, the aptamer has a sequence composition where at least 90% of all guanosine nucleotides are 2′-OH guanosine, at least 90% of all cytidine nucleotides are 2′-O-methyl cytidine, at least 90% of all uridine nucleotides are 2′-O-methyl uridine, and at least 90% of all adenosine nucleotides are 2′-O-methyl adenosine. In another embodiment, the aptamer has a sequence composition where 100% of all guanosine nucleotides are 2′-OH guanosine, 100% of all cytidine nucleotides are 2′-O-methyl cytidine, 100% of all uridine nucleotides are 2′-O-methyl uridine, and 100% of all adenosine nucleotides are 2′-O-methyl adenosine.

The invention also relates to an aptamer composition comprising a sequence where substantially all adenosine nucleotides are 2′-O-methyl adenosine, substantially all cytidine nucleotides are 2′-O-methyl cytidine, substantially all guanosine nucleotides are 2′-O-methyl guanosine or deoxy guanosine, substantially all uridine nucleotides are 2′-O-methyl uridine, where less than about 10% of the guanosine nucleotides are deoxy guanosine. In one embodiment, the aptamer has a sequence composition where at least 80% of all adenosine nucleotides are 2′-O-methyl adenosine, at least 80% of all cytidine nucleotides are 2′-O-methyl cytidine, at least 80% of all guanosine nucleotides are 2′-O-methyl guanosine, at least 80% of all uridine nucleotides are 2′-O-methyl uridine, and no more than about 10% of all guanosine nucleotides are deoxy guanosine. In another embodiment, the aptamer has a sequence composition where at least 90% of all adenosine nucleotides are 2′-O-methyl adenosine, at least 90% of all cytidine nucleotides are 2′-O-methyl cytidine, at least 90% of all guanosine nucleotides are 2′-O-methyl guanosine, at least 90% of all uridine nucleotides are 2′-O-methyl uridine, and no more than about 10% of all guanosine nucleotides are deoxy guanosine. In another embodiment, the aptamer has a sequence composition where 100% of all adenosine nucleotides are 2′-O-methyl adenosine, 100% of all cytidine nucleotides are 2′-O-methyl cytidine, at least 90% of all guanosine nucleotides are 2′-O-methyl guanosine, 100% of all uridine nucleotides are 2′-O-methyl uridine, and no more than about 10% of all guanosine nucleotides are deoxy guanosine.

The invention also relates to an aptamer composition comprising a sequence where substantially all adenosine nucleotides are 2′-O-methyl adenosine, substantially all uridine nucleotides are 2′-O-methyl uridine, substantially all cytidine nucleotides are 2′-O-methyl cytidine, and substantially all guanosine nucleotides are 2′-F guanosine sequence. In one embodiment, the aptamer has a sequence composition where at least 80% of all adenosine nucleotides are 2′-O-methyl adenosine, at least 80% of all uridine nucleotides are 2′-O-methyl uridine, at least 80% of all cytidine nucleotides are 2′-O-methyl cytidine, and at least 80% of all guanosine nucleotides are 2′-F guanosine. In another embodiment, the aptamer has a sequence composition where at least 90% of all adenosine nucleotides are 2′-O-methyl adenosine, at least 90% of all uridine nucleotides are 2′-O-methyl uridine, at least 90% of all cytidine nucleotides are 2′-O-methyl cytidine, and at least 90% of all guanosine nucleotides are 2′-F guanosine. In another embodiment, the aptamer has a sequence composition where 100% of all adenosine nucleotides are 2′-O-methyl adenosine, 100% of all uridine nucleotides are 2′-O-methyl uridine, 100% of all cytidine nucleotides are 2′-O-methyl cytidine, and 100% of all guanosine nucleotides are 2′-F guanosine.

The invention also relates to an aptamer composition comprising a sequence where substantially all adenosine nucleotides are 2′-deoxy adenosine, substantially all cytidine nucleotides are 2′-O-methyl cytidine, substantially all guanosine nucleotides are 2′-O-methyl guanosine, and substantially all uridine nucleotides are 2′-O-methyl uridine. In one embodiment, the aptamer has a sequence composition where at least 80% of all adenosine nucleotides are 2′-deoxy adenosine, at least 80% of all cytidine nucleotides are 2′-O-methyl cytidine, at least 80% of all guanosine nucleotides are 2′-O-methyl guanosine, and at least 80% of all uridine nucleotides are 2′-O-methyl uridine. In another embodiment, the aptamer has a sequence composition where at least 90% of all adenosine nucleotides are 2′-deoxy adenosine, at least 90% of all cytidine nucleotides are 2′-O-methyl cytidine, at least 90% of all guanosine nucleotides are 2′-O-methyl guanosine, and at least 90% of all uridine nucleotides are 2′-O-methyl uridine. In another embodiment, the aptamer has a sequence composition where 100% of all adenosine nucleotides are 2′-deoxy adenosine, 100% of all cytidine nucleotides are 2′-O-methyl cytidine, 100% of all guanosine nucleotides are 2′-O-methyl guanosine, and 100% of all uridine nucleotides are 2′-O-methyl uridine.

The invention also relates to an aptamer composition comprising a sequence where substantially all adenosine nucleotides are 2′-OH adenosine, substantially all guanosine nucleotides are 2′-OH guanosine, substantially all cytidine nucleotides are 2′-OH cytidine, and substantially all uridine nucleotides are 2′-OH uridine. In one embodiment, the aptamer has a sequence composition where at least 80% of all adenosine nucleotides are 2′-OH adenosine, at least 80% of all cytidine nucleotides are 2′-OH cytidine, at least 80% of all guanosine nucleotides are 2′-OH guanosine, and at least 80% of all uridine nucleotides are 2′-OH uridine. In another embodiment, the aptamer has a sequence composition where at least 90% of all adenosine nucleotides are 2′-OH adenosine, at least 90% of all cytidine nucleotides are 2′-OH cytidine, at least 90% of all guanosine nucleotides are 2′-OH guanosine, and at least 90% of all uridine nucleotides are 2′-OH uridine. In another embodiment, the aptamer has a sequence composition where 100% of all adenosine nucleotides are 2′-OH adenosine, 100% of all cytidine nucleotides are 2′-OH cytidine, 100% of all guanosine nucleotides are 2′-OH guanosine, and 100% of all uridine nucleotides are 2′-OH uridine.

### DETAILED DESCRIPTION OF THE INVENTION

The details of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description. In the specification, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present Specification will control.

Modified Nucleotide Transcription

The present invention provides materials and methods to produce stabilized oligonucleotides (including, e.g., aptamers) that contain modified nucleotides (e.g., nucleotides which have a modification at the 2′position) which make the oligonucleotide more stable than the unmodified oligonucleotide. The stabilized oligonucleotides produced by the materials and methods of the present invention are also more stable to enzymatic and chemical degradation as well as thermal and physical degradation.

In order for an aptamer to be suitable for use as a therapeutic, it is preferably inexpensive to synthesize, safe and stable in vivo. Wild-type RNA and DNA aptamers are typically not stable in vivo because of their susceptibility to degradation by nucleases. Resistance to nuclease degradation can be greatly increased by the incorporation of modifying groups at the 2′-position. Fluoro and amino groups have been successfully incorporated into oligonucleotide libraries from which aptamers have been subsequently selected. However, these modifications greatly increase the cost of synthesis of the resultant aptamer, and may introduce safety concerns because of the possibility that the modified nucleotides could be recycled into host DNA, by degradation of the modified oligonucleotides and subsequent use of the nucleotides as substrates for DNA synthesis.

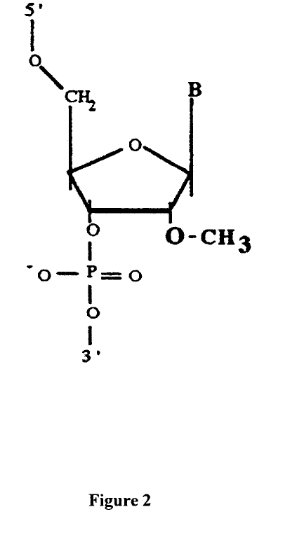

Aptamers that contain 2′-O-methyl (2′-OMe) nucleotides overcome many of these drawbacks. Oligonucleotides containing 2′-O-methyl nucleotides are nuclease-resistant and inexpensive to synthesize. Although 2′-O-methyl nucleotides are ubiquitous in biological systems, natural polymerases do not accept 2′-O-methyl NTPs as substrates under physiological conditions, thus there are no safety concerns over the recycling of 2′-O-methyl nucleotides into host DNA. A generic formula for a 2′-OMe nucleotide is shown in FIG. 2.

There are several examples of 2′-O-Mecontaining aptamers in the literature, see, for example Green et al., Current Biology 2, 683-695, 1995. These were generated by the in vitro selection of libraries of modified transcripts in which the C and U residues were 2′-fluoro (2′-F) substituted and the A and G residues were 2′-OH. Once functional sequences were identified then each A and G residue was tested for tolerance to 2′-OMe substitution, and the aptamer was re-synthesized having all A and G residues which tolerated 2′-OMe substitution as 2′-OMe residues. Most of the A and G residues of aptamers generated in this two-step fashion tolerate substitution with 2′-OMe residues, although, on average, approximately 20% do not. Consequently, aptamers generated using this method tend to contain from two to four 2′-OH residues, and stability and cost of synthesis are compromised as a result. By incorporating modified nucleotides into the transcription reaction which generate stabilized oligonucleotides used in oligonucleotide libraries from which aptamers are selected and enriched by SELEX™ (and/or any of its variations and improvements, including those described below), the methods of the current invention eliminate the need for stabilizing the selected aptamer oligonucleotides (e.g., by resynthesizing the aptamer oligonucleotides with modified nucleotides).

Furthermore, the modified oligonucleotides of the invention can be further stabilized after the selection process has been completed. (See “post-SELEX™ modifications”, including truncating, deleting and modification, below.)

The SELEX™ Method

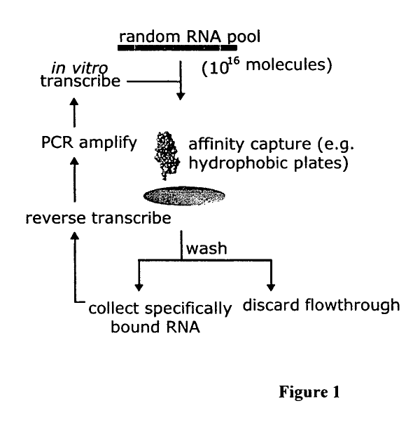

A suitable method for generating an aptamer is with the process entitled “Systematic Evolution of Ligands by EXponential enrichment” (“SELEX™”) depicted generally in FIG. 1. The SELEX™ process is a method for the in vitro evolution of nucleic acid molecules with highly specific binding to target molecules and is described in, e.g., U.S. patent application Ser. No. 07/536,428, filed Jun. 11, 1990, now abandoned, U.S. Pat. No. 5,475,096 entitled “Nucleic Acid Ligands”, and U.S. Pat. No. 5,270,163 (see also WO 91/19813) entitled “Nucleic Acid Ligands”. Each SELEX™-identified nucleic acid ligand is a specific ligand of a given target compound or molecule. The SELEX™ process is based on the unique insight that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets.

SELEX™ relies as a starting point upon a large library of single stranded oligonucleotide templates comprising randomized sequences derived from chemical synthesis on a standard DNA synthesizer. In some examples, a population of 100% random oligonucleotides is screened. In others, each oligonucleotide in the population comprises a random sequence and at least one fixed sequence at its 5′ and/or 3′ end which comprises a sequence shared by all the molecules of the oligonucleotide population. Fixed sequences include sequences such as hybridization sites for PCR primers, promoter sequences for RNA polymerases (e.g., T3, T4, T7, SP6, and the like), restriction sites, or homopolymeric sequences, such as poly A or poly T tracts, catalytic cores, sites for selective binding to affinity columns, and other sequences to facilitate cloning and/or sequencing of an oligonucleotide of interest.

The random sequence portion of the oligonucleotide can be of any length and can comprise ribonucleotides and/or deoxyribonucleotides and can include modified or non-natural nucleotides or nucleotide analogs. See, e.g., U.S. Pat. Nos. 5,958,691; 5,660,985; 5,958,691; 5,698,687; 5,817,635; and 5,672,695, and PCT publication WO 92/07065. Random oligonucleotides can be synthesized from phosphodiester-linked nucleotides using solid phase oligonucleotide synthesis techniques well known in the art (Froehler et al., Nucl. Acid Res. 14:5399-5467 (1986); Froehler et al., Tet. Lett. 27:5575-5578 (1986)). Oligonucleotides can also be synthesized using solution phase methods such as triester synthesis methods (Sood et al., Nucl. Acid Res. 4:2557 (1977); Hirose et al., Tet. Lett., 28:2449 (1978)). Typical syntheses carried out on automated DNA synthesis equipment yield 10¹⁵-10¹⁷ molecules. Sufficiently large regions of random sequence in the sequence design increases the likelihood that each synthesized molecule is likely to represent a unique sequence.

To synthesize randomized sequences, mixtures of all four nucleotides are added at each nucleotide addition step during the synthesis process, allowing for random incorporation of nucleotides. In one embodiment, random oligonucleotides comprise entirely random sequences; however, in other embodiments, random oligonucleotides can comprise stretches of nonrandom or partially random sequences. Partially random sequences can be created by adding the four nucleotides in different molar ratios at each addition step.

Template molecules typically contain fixed 5′ and 3′ terminal sequences which flank an internal region of 30-50 random nucleotides. A standard (1 μmole) scale synthesis will yield 10¹⁵-10¹⁶ individual template molecules, sufficient for most SELEX™ experiments. The RNA library is generated from this starting library by in vitro transcription using recombinant T7 RNA polymerase. This library is then mixed with the target under conditions favorable for binding and subjected to step-wise iterations of binding, partitioning and amplification, using the same general selection scheme, to achieve virtually any desired criterion of binding affinity and selectivity. Starting from a mixture of nucleic acids, preferably comprising a segment of randomized sequence, the SELEX™ method includes steps of contacting the mixture with the target under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand-enriched mixture of nucleic acids, then reiterating the steps of binding, partitioning, dissociating and amplifying through as many cycles as desired to yield highly specific high affinity nucleic acid ligands to the target molecule.

Within a nucleic acid mixture containing a large number of possible sequences and structures, there is a wide range of binding affinities for a given target. A nucleic acid mixture comprising, for example a 20 nucleotide randomized segment containing only natural unmodified nucleotides can have 420 candidate possibilities. Those which have the higher affinity constants for the target are most likely to bind to the target. After partitioning, dissociation and amplification, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands.

Cycles of selection and amplification are repeated until a desired goal is achieved. In the most general case, selection/amplification is continued until no significant improvement in binding strength is achieved on repetition of the cycle. The method may be used to sample as many as about 10¹⁸ different nucleic acid species. The nucleic acids of the test mixture preferably include a randomized sequence portion as well as conserved sequences necessary for efficient amplification. Nucleic acid sequence variants can be produced in a number of ways including synthesis of randomized nucleic acid sequences and size selection from randomly cleaved cellular nucleic acids. The variable sequence portion may contain fully or partially random sequence; it may also contain subportions of conserved sequence incorporated with randomized sequence. Sequence variation in test nucleic acids can be introduced or increased by mutagenesis before or during the selection/amplification iterations.

In one embodiment of SELEX™, the selection process is so efficient at isolating those nucleic acid ligands that bind most strongly to the selected target, that only one cycle of selection and amplification is required. Such an efficient selection may occur, for example, in a chromatographic-type process wherein the ability of nucleic acids to associate with targets bound on a column operates in such a manner that the column is sufficiently able to allow separation and isolation of the highest affinity nucleic acid ligands.

In many cases, it is not necessarily desirable to perform the iterative steps of SELEX™ until a single nucleic acid ligand is identified. The target-specific nucleic acid ligand solution may include a family of nucleic acid structures or motifs that have a number of conserved sequences and a number of sequences which can be substituted or added without significantly affecting the affinity of the nucleic acid ligands to the target. By terminating the SELEX™ process prior to completion, it is possible to determine the sequence of a number of members of the nucleic acid ligand solution family.

A variety of nucleic acid primary, secondary and tertiary structures are known to exist. The structures or motifs that have been shown most commonly to be involved in non-Watson-Crick type interactions are referred to as hairpin loops, symmetric and asymmetric bulges, pseudoknots and myriad combinations of the same. Almost all known cases of such motifs suggest that they can be formed in a nucleic acid sequence of no more than 30 nucleotides. For this reason, it is often preferred that SELEX™ procedures with contiguous randomized segments be initiated with nucleic acid sequences containing a randomized segment of between about 20-50 nucleotides.

The core SELEX™ method has been modified to achieve a number of specific objectives. For example, U.S. Pat. No. 5,707,796 describes the use of SELEX™ in conjunction with gel electrophoresis to select nucleic acid molecules with specific structural characteristics, such as bent DNA. U.S. Pat. No. 5,763,177 describes SELEX™ based methods for selecting nucleic acid ligands containing photoreactive groups capable of binding and/or photocrosslinking to and/or photoinactivating a target molecule. U.S. Pat. No. 5,567,588 and U.S. application Ser. No. 08/792,075, filed Jan. 31, 1997, entitled “Flow Cell SELEX™”, describe SELEX™ based methods which achieve highly efficient partitioning between oligonucleotides having high and low affinity for a target molecule. U.S. Pat. No. 5,496,938 describes methods for obtaining improved nucleic acid ligands after the SELEX™ process has been performed. U.S. Pat. No. 5,705,337 describes methods for covalently linking a ligand to its target.

SELEX™ can also be used to obtain nucleic acid ligands that bind to more than one site on the target molecule, and to obtain nucleic acid ligands that include non-nucleic acid species that bind to specific sites on the target. SELEX™ provides means for isolating and identifying nucleic acid ligands which bind to any envisionable target, including large and small biomolecules including proteins (including both nucleic acid-binding proteins and proteins not known to bind nucleic acids as part of their biological function) cofactors and other small molecules. For example, see U.S. Pat. No. 5,580,737 which discloses nucleic acid sequences identified through SELEX™ which are capable of binding with high affinity to caffeine and the closely related analog, theophylline.

Counter-SELEX™ is a method for improving the specificity of nucleic acid ligands to a target molecule by eliminating nucleic acid ligand sequences with cross-reactivity to one or more non-target molecules. Counter-SELEX™ is comprised of the steps of a) preparing a candidate mixture of nucleic acids; b) contacting the candidate mixture with the target, wherein nucleic acids having an increased affinity to the target relative to the candidate mixture may be partitioned from the remainder of the candidate mixture; c) partitioning the increased affinity nucleic acids from the remainder of the candidate mixture; d) contacting the increased affinity nucleic acids with one or more non-target molecules such that nucleic acid ligands with specific affinity for the non-target molecule(s) are removed; and e) amplifying the nucleic acids with specific affinity to the target molecule to yield a mixture of nucleic acids enriched for nucleic acid sequences with a relatively higher affinity and specificity for binding to the target molecule.

One potential problem encountered in the use of nucleic acids as therapeutics and vaccines is that oligonucleotides in their phosphodiester form may be quickly degraded in body fluids by intracellular and/or extracellular enzymes such as endonucleases and exonucleases before the desired effect is manifest. SELEX™ methods therefore encompass the identification of high-affinity nucleic acid ligands which are altered, after selection, to contain modified nucleotides which confer improved characteristics on the ligand, such as improved in vivo stability or improved delivery characteristics. Modifications of nucleic acid ligands include, but are not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole. Modifications include chemical substitutions at the ribose and/or phosphate and/or base positions, such as 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations, unusual base-pairing combinations such as the isobases isocytidine and isoguanidine and the like. Modifications can also include 3′ and 5′ modifications such as capping.

In oligonucleotides which comprise modified sugar groups, for example, one or more of the hydroxyl groups is replaced with halogen, aliphatic groups, or functionalized as ethers or amines. Examples of substitution at the 2′-posititution of the furanose residue include O-alkyl (e.g., O-methyl), O-allyl, S-alkyl, S-allyl, or a halo group. Methods of synthesis of 2′-modified sugars are described in Sproat, et al., Nucl. Acid Res. 19:733-738 (1991); Cotten, et al., Nucl. Acid Res. 19:2629-2635 (1991); and Hobbs, et al., Biochemistry 12:5138-5145 (1973). Other modifications are known to one of ordinary skill in the art.

SELEX™-identified nucleic acid ligands synthesized after selection to contain modified nucleotides are described in U.S. Pat. No. 5,660,985, which describes oligonucleotides containing nucleotide derivatives chemically modified at the 5′ and 2′ positions of pyrimidines. Additionally, U.S. Pat. No. 5,756,703 describes oligonucleotides containing various 2′-modified pyrimidines; and U.S. Pat. No. 5,580,737 describes highly specific nucleic acid ligands containing one or more nucleotides modified with 2′-amino (2′-NH₂), 2′-fluoro (2′-F), and/or 2′-O-methyl (2′-OMe) substituents.

The SELEX™ method encompasses combining selected oligonucleotides with other selected oligonucleotides and non-oligonucleotide functional units as described in U.S. Pat. No. 5,637,459 and U.S. Pat. No. 5,683,867. The SELEX™ method further encompasses combining selected nucleic acid ligands with lipophilic or non-immunogenic high molecular weight compounds in a diagnostic or therapeutic complex, as described in U.S. Pat. No. 6,011,020. VEGF nucleic acid ligands that are associated with a lipophilic compound, such as diacyl glycerol or dialkyl glycerol, in a diagnostic or therapeutic complex are described in U.S. Pat. No. 5,859,228.

VEGF nucleic acid ligands that are associated with a lipophilic compound, such as a glycerol lipid, or a non-immunogenic high molecular weight compound, such as polyalkylene glycol are further described in U.S. Pat. No. 6,051,698. VEGF nucleic acid ligands that are associated with a non-immunogenic, high molecular weight compound or a lipophilic compound are further described in PCT Publication No. WO 98/18480. These patents and applications describe the combination of a broad array of oligonucleotide shapes and other properties, and the efficient amplification and replication properties, of oligonucleotides with the desirable properties of other molecules.

The identification of nucleic acid ligands to small, flexible peptides via the SELEX™ method has also been explored. Small peptides have flexible structures and usually exist in solution in an equilibrium of multiple conformers, and thus it was initially thought that binding affinities may be limited by the conformational entropy lost upon binding a flexible peptide. However, the feasibility of identifying nucleic acid ligands to small peptides in solution was demonstrated in U.S. Pat. No. 5,648,214. In this patent, high affinity RNA nucleic acid ligands to substance P, an 11 amino acid peptide, were identified.

To generate oligonucleotide populations which are resistant to nucleases and hydrolysis, modified oligonucleotides can be used and can include one or more substitute internucleotide linkages, altered sugars, altered bases, or combinations thereof. In one embodiment, oligonucleotides are provided in which the P(O)O group is replaced by P(O)S (“thioate”), P(S)S (“dithioate”), P(O)NR₂ (“amidate”), P(O)R, P(O)OR′, CO or CH₂ (“formacetal”) or 3′-amine (—NH—CH₂—CH₂—), wherein each R or R′ is independently H or substituted or unsubstituted alkyl. Linkage groups can be attached to adjacent nucleotide through an —O—, —N—, or —S— linkage. Not all linkages in the oligonucleotide are required to be identical.

Nucleic acid aptamer molecules are generally selected in a 5 to 20 cycle procedure. In one embodiment, heterogeneity is introduced only in the initial selection stages and does not occur throughout the replicating process.

The starting library of DNA sequences is generated by automated chemical synthesis on a DNA synthesizer. This library of sequences is transcribed in vitro into RNA using T7 RNA polymerase or a modified T7 RNA polymerase, and purified. In one example, the 5′-fixed:random:3′-fixed sequence includes a random sequence having from 30 to 50 nucleotides.

Incorporation of modified nucleotides into the aptamers of the invention is accomplished before (pre-) the selection process (e.g., a pre-SELEX™ process modification). Optionally, aptamers of the invention in which modified nucleotides have been incorporated by pre-SELEX™ process modification can be further modified by post-SELEX™ process modification (i.e., a post-SELEX™ process modification after a pre-SELEX™ modification). Pre-SELEX™ process modifications yield modified nucleic acid ligands with specificity for the SELEX™ target and also improved in vivo stability. Post-SELEX™ process modifications (e.g., modification of previously identified ligands having nucleotides incorporated by pre-SELEX™ process modification) can result in a further improvement of in vivo stability without adversely affecting the binding capacity of the nucleic acid ligand having nucleotides incorporated by pre-SELEX™ process modification.

Modified Polymerases

A single mutant T7 polymerase (Y639F) in which the tyrosine residue at position 639 has been changed to phenylalanine readily utilizes 2′deoxy, 2′amino-, and 2′fluoro-nucleotide triphosphates (NTPs) as substrates and has been widely used to synthesize modified RNAs for a variety of applications. However, this mutant T7 polymerase reportedly can not readily utilize (e.g., incorporate) NTPs with bulkier 2′-substituents, such as 2′-O-methyl (2′-OMe) or 2′-azido (2′-N₃) substituents. For incorporation of bulky 2′ substituents, a double T7 polymerase mutant (Y639F/H784A) having the histidine at position 784 changed to an alanine, or other small amino acid, residue, in addition to the Y639F mutation has been described and has been used to incorporate modified pyrimidine NTPs. A single mutant T7 polymerase (H784A) having the histidine at position 784 changed to an alanine residue has also been described. (Padilla et al., Nucleic Acids Research, 2002, 30: 138). In both the Y639F/H784A double mutant and H784A single mutant T7 polymerases, the change to smaller amino acid residues allows for the incorporation of bulkier nucleotide substrates, e.g., 2′-O methyl substituted nucleotides.

The present invention provides methods and conditions for using these and other modified T7 polymerases having a higher incorporation rate of modified nucleotides having bulky substituents at the furanose 2′ position, than wild-type polymerases. Generally, it has been found that under the conditions disclosed herein, the Y693F single mutant can be used for the incorporation of all 2′-OMe substituted NTPs except GTP and the Y639F/H784A double mutant can be used for the incorporation of all 2′-OMe substituted NTPs including GTP. It is expected that the H784A single mutant possesses similar properties when used under the conditions disclosed herein.

The present invention provides methods and conditions for modified T7 polymerases to enzymatically incorporate modified nucleotides into oligonucleotides. Such oligonucleotides may be synthesized entirely of modified nucleotides, or with a subset of modified nucleotides. The modifications can be the same or different. All nucleotides may be modified, and all may contain the same modification. All nucleotides may be modified, but contain different modifications, e.g., all nucleotides containing the same base may have one type of modification, while nucleotides containing other bases may have different types of modification. All purine nucleotides may have one type of modification (or are unmodified), while all pyrimidine nucleotides have another, different type of modification (or are unmodified). In this way, transcripts, or libraries of transcripts are generated using any combination of modifications, for example, ribonucleotides, (2′-OH, “rN”), deoxyribonucleotides (2′-deoxy), 2′-F, and 2′-OMe nucleotides. A mixture containing 2′-OMe C and U and 2′-OH A and G is called “rRmY”; a mixture containing deoxy A and G and 2′-OMe U and C is called “dRmY”; a mixture containing 2′-OMe A, C, and U, and 2′-OH G is called “rGmH”; a mixture alternately containing 2′-OMe A, C, U and G and 2′-OMe A, U and C and 2′-F G is called “toggle”; a mixture containing 2′-OMe A, U, C, and G, where up to 10% of the G's are deoxy is called “r/mGmH”; a mixture containing 2′-O Me A, U, and C, and 2′-F G is called “fGmH”; and a mixture containing deoxy A, and 2′-OMe C, G and U is called “dAmB”.

A preferred embodiment includes any combination of 2′-OH, 2′-deoxy and 2′-OMe nucleotides. A more preferred embodiment includes any combination of 2′-deoxy and 2′-OMe nucleotides. An even more preferred embodiment is with any combination of 2′-deoxy and 2′-OMe nucleotides in which the pyrimidines are 2′-OMe (such as dRmY, mN or dGmH).

2′-Modified SELEX™

The present invention provides methods to generate libraries of 2′-modified (e.g., 2′-OMe) RNA transcripts in conditions under which a polymerase accepts 2′-modified NTPs. Preferably, the polymerase is the Y693F/H784A double mutant or the Y693F single mutant. Other polymerases, particularly those that exhibit a high tolerance for bulky 2′-substituents, may also be used in the present invention. Such polymerases can be screened for this capability by assaying their ability to incorporate modified nucleotides under the transcription conditions disclosed herein. A number of factors have been determined to be crucial for the transcription conditions useful in the methods disclosed herein. For example, great increases in the yields of modified transcript are observed when a leader sequence is incorporated into the 5′ end of a fixed sequence at the 5′ end of the DNA transcription template, such that at least about the first 6 residues of the resultant transcript are all purines.

Another important factor in obtaining transcripts incorporating modified nucleotides is the presence or concentration of 2′-OH GTP. Transcription can be divided into two phases: the first phase is initiation, during which an NTP is added to the 3′-hydroxyl end of GTP (or another substituted guanosine) to yield a dinucleotide which is then extended by about 10-12 nucleotides, the second phase is elongation, during which transcription proceeds beyond the addition of the first about 10-12 nucleotides. It has been found that small amounts of 2′-OH GTP added to a transcription mixture containing an excess of 2′-OMe GTP are sufficient to enable the polymerase to initiate transcription using 2′-OH GTP, but once transcription enters the elongation phase the reduced discrimination between 2′-OMe and 2′-OH GTP, and the excess of 2′-OMe GTP over 2′-OH GTP allows the incorporation of principally the 2′-OMe GTP.

Another important factor in the incorporation of 2′-OMe into transcripts is the use of both divalent magnesium and manganese in the transcription mixture. Different combinations of concentrations of magnesium chloride and manganese chloride have been found to affect yields of 2′-O-methylated transcripts, the optimum concentration of the magnesium and manganese chloride being dependent on the concentration in the transcription reaction mixture of NTPs which complex divalent metal ions. To obtain the greatest yields of maximally 2′ substituted O-methylated transcripts (i.e., all A, C, and U and about 90% of G nucleotides), concentrations of approximately 5 mM magnesium chloride and 1.5 mM manganese chloride are preferred when each NTP is present at a concentration of 0.5 mM. When the concentration of each NTP is 1.0 mM, concentrations of approximately 6.5 mM magnesium chloride and 2.0 mM manganese chloride are preferred. When the concentration of each NTP is 2.0 mM, concentrations of approximately 9.6 mM magnesium chloride and 2.9 mM manganese chloride are preferred. In any case, departures from these concentrations of up to two-fold still give significant amounts of modified transcripts.

Priming transcription with GMP or guanosine is also important. This effect results from the specificity of the polymerase for the initiating nucleotide. As a result, the 5′-terminal nucleotide of any transcript generated in this fashion is likely to be 2′-OH G. The preferred concentration of GMP (or guanosine) is 0.5 mM and even more preferably 1 mM. It has also been found that including PEG, preferably PEG-8000, in the transcription reaction is useful to maximize incorporation of modified nucleotides.

For maximum incorporation of 2′-OMe ATP (100%), UTP(100%), CTP(100%) and GTP (˜90%) (“r/mGmH”) into transcripts the following conditions are preferred: HEPES buffer 200 mM, DTT 40 mM, spermidine 2 mM, PEG-8000 10% (w/v), Triton X-100 0.01% (w/v), MgCl₂ 5 mM (6.5 mM where the concentration of each 2′-OMe NTP is 1.0 mM), MnCl₂ 1.5 mM (2.0 mM where the concentration of each 2′-OMe NTP is 1.0 mM), 2′-OMe NTP (each) 500 μM (more preferably, 1.0 mM), 2′-OH GTP 30 μM, 2′-OH GMP 500 μM, pH 7.5, Y639F/H784A T7 RNA Polymerase 15 units/ml, inorganic pyrophosphatase 5 units/ml, and an all-purine leader sequence of at least 8 nucleotides long. As used herein, one unit of the Y639F/H784A mutant T7 RNA polymerase, or any other mutant T7 RNA polymerase specified herein) is defined as the amount of enzyme required to incorporate 1 nmole of 2′-OMe NTPs into transcripts under the r/mGmH conditions. As used herein, one unit of inorganic pyrophosphatase is defined as the amount of enzyme that will liberate 1.0 mole of inorganic orthophosphate per minute at pH 7.2 and 25° C.

For maximum incorporation (100%) of 2′-OMe ATP, UTP and CTP (“rGmH”) into transcripts the following conditions are preferred: HEPES buffer 200 mM, DTT 40 mM, spermidine 2 mM, PEG-8000 10% (w/v), Triton X-100 0.01% (w/v), MgCl₂ 5 mM (9.6 mM where the concentration of each 2′-OMe NTP is 2.0 mM), MnCl₂ 1.5 mM (2.9 mM where the concentration of each 2′-OMe NTP is 2.0 mM), 2′-OMe NTP (each) 500 μM (more preferably, 2.0 mM), pH 7.5, Y639F T7 RNA Polymerase 15 units/ml, inorganic pyrophosphatase 5 units/ml, and an all-purine leader sequence of at least 8 nucleotides long.

For maximum incorporation (100%) of 2′-OMe UTP and CTP (“rRmY”) into transcripts the following conditions are preferred: HEPES buffer 200 mM, DTT 40 mM, spermidine 2 mM, PEG-8000 10% (w/v), Triton X-100 0.01% (w/v), MgCl₂ 5 mM (9.6 mM where the concentration of each 2′-OMe NTP is 2.0 mM), MnCl₂ 1.5 mM (2.9 mM where the concentration of each 2′-OMe NTP is 2.0 mM), 2′-OMe NTP (each) 500 μM (more preferably, 2.0 mM), pH 7.5, Y639F/H784A T7 RNA Polymerase 15 units/ml, inorganic pyrophosphatase 5 units/ml, and an all-purine leader sequence of at least 8 nucleotides long.

For maximum incorporation (100%) of deoxy ATP and GTP and 2′-OMe UTP and CTP (“dRmY”) into transcripts the following conditions are preferred: HEPES buffer 200 mM, DTT 40 mM, spermidine 2 mM, PEG-8000 10% (w/v), Triton X-100 0.01% (w/v), MgCl₂ 9.6 mM, MnCl₂ 2.9 mM, 2′-OMe NTP (each) 2.0 mM, pH 7.5, Y639F T7 RNA Polymerase 15 units/ml, inorganic pyrophosphatase 5 units/ml, and an all-purine leader sequence of at least 8 nucleotides long.

For maximum incorporation (100%) of 2′-OMe ATP, UTP and CTP and 2′-F GTP (“fGmH”) into transcripts the following conditions are preferred: HEPES buffer 200 mM, DTT 40 mM, spermidine 2 mM, PEG-8000 10% (w/v), Triton X-100 0.01% (w/v), MgCl₂ 9.6 mM, MnCl₂ 2.9 mM, 2′-OMe NTP (each) 2.0 mM, pH 7.5, Y639F T7 RNA Polymerase 15 units/ml, inorganic pyrophosphatase 5 units/ml, and an all-purine leader sequence of at least 8 nucleotides long.

For maximum incorporation (100%) of deoxy ATP and 2′-OMe UTP, GTP and CTP (“dAmB”) into transcripts the following conditions are preferred: HEPES buffer 200 mM, DTT 40 mM, spermidine 2 mM, PEG-8000 10% (w/v), Triton X-100 0.01% (w/v), MgCl₂ 9.6 mM, MnCl₂ 2.9 mM, 2′-OMe NTP (each) 2.0 mM, pH 7.5, Y639F T7 RNA Polymerase 15 units/ml, inorganic pyrophosphatase 5 units/ml, and an all-purine leader sequence of at least 8 nucleotides long.

For each of the above, (1) transcription is preferably performed at a temperature of from about 30° C. to about 45° C. and for a period of at least two hours and (2) 50-300 nM of a double stranded DNA transcription template is used (200 nm template was used for round 1 to increase diversity (300 nm template was used for dRmY transcriptions), and for subsequent rounds approximately 50 nM, a 1/10 dilution of an optimized PCR reaction, using conditions described herein, was used). The preferred DNA transcription templates are described below (where ARC254 and ARC256 transcribe under all 2′-OMe conditions and ARC255 transcribes under rRmY conditions). ARC254:

| | |
|---|---|
| ARC254: | |
| 5′-CATCGATGCTAGTCGTAACGATCCNNNNNNN | (SEQ ID NO:1) |
| NNNNNNNNNNNNNNNNNNNNNNNCGAGAACGTTC | |
| TCTCCTCTCCCTATAGTGAGTCGTATTA-3′ | |
| ARC255: | |
| 5′-CATGCATCGCGACTGACTAGCCGNNNNNNNN | (SEQ ID NO:2) |
| NNNNNNNNNNNNNNNNNNNNNNGTAGAACGTTCT | |
| CTCCTCTCCCTATAGTGAGTCGTATTA-3′ | |
| ARC256: | |
| 5′-CATCGATCGATCGATCGACAGCGNNNNNNNN | (SEQ ID NO:453) |
| NNNNNNNNNNNNNNNNNNNNNNGTAGAACGTTCT | |
| CTCCTCTCCCTATAGTGAGTCGTATTA-3′ | |

Under rN transcription conditions of the present invention, the transcription reaction mixture comprises 2′-OH adenosine triphosphates (ATP), 2′-OH guanosine triphosphates (GTP), 2′-OH cytidine triphosphates (CTP), and 2′-OH uridine triphosphates (UTP). The modified oligonucleotides produced using the rN transcription mixtures of the present invention comprise substantially all 2′-OH adenosine, 2′-OH guanosine, 2′-OH cytidine, and 2′-OH uridine. In a preferred embodiment of rN transcription, the resulting modified oligonucleotides comprise a sequence where at least 80% of all adenosine nucleotides are 2′-OH adenosine, at least 80% of all guanosine nucleotides are 2′-OH guanosine, at least 80% of all cytidine nucleotides are 2′-OH cytidine, and at least 80% of all uridine nucleotides are 2′-OH uridine. In a more preferred embodiment of rN transcription, the resulting modified oligonucleotides of the present invention comprise a sequence where at least 90% of all adenosine nucleotides are 2′-OH adenosine, at least 90% of all guanosine nucleotides are 2′-OH guanosine, at least 90% of all cytidine nucleotides are 2′-OH cytidine, and at least 90% of all uridine nucleotides are 2′-OH uridine. In a most preferred embodiment of rN transcription, the modified oligonucleotides of the present invention comprise 100% of all adenosine nucleotides are 2′-OH adenosine, of all guanosine nucleotides are 2′-OH guanosine, of all cytidine nucleotides are 2′-OH cytidine, and of all uridine nucleotides are 2′-OH uridine.

Under rRmY transcription conditions of the present invention, the transcription reaction mixture comprises 2′-OH adenosine triphosphates, 2′-OH guanosine triphosphates, 2′-O-methyl cytidine triphosphates, and 2′-O-methyl uridine triphosphates. The modified oligonucleotides produced using the rRmY transcription mixtures of the present invention comprise substantially all 2′-OH adenosine, 2′-OH guanosine, 2′-O-methyl cytidine and 2′-O-methyl uridine. In a preferred embodiment, the resulting modified oligonucleotides comprise a sequence where at least 80% of all adenosine nucleotides are 2′-OH adenosine, at least 80% of all guanosine nucleotides are 2′-OH guanosine, at least 80% of all cytidine nucleotides are 2′-O-methyl cytidine and at least 80% of all uridine nucleotides are 2′-O-methyl uridine. In a more preferred embodiment, the resulting modified oligonucleotides comprise a sequence where at least 90% of all adenosine nucleotides are 2′-OH adenosine, at least 90% of all guanosine nucleotides are 2′-OH guanosine, at least 90% of all cytidine nucleotides are 2′-O-methyl cytidine and at least 90% of all uridine nucleotides are 2′-O-methyl uridine In a most preferred embodiment, the resulting modified oligonucleotides comprise a sequence where 100% of all adenosine nucleotides are 2′-OH adenosine, 100% of all guanosine nucleotides are 2′-OH guanosine, 100% of all cytidine nucleotides are 2′-O-methyl cytidine and 100% of all uridine nucleotides are 2′-O-methyl uridine.

Under dRmY transcription conditions of the present invention, the transcription reaction mixture comprises 2′-deoxy purine triphosphates and 2′-O-methylpyrimidine triphosphates. The modified oligonucleotides produced using the dRmY transcription conditions of the present invention comprise substantially all 2′-deoxy purines and 2′-O-methyl pyrimidines. In a preferred embodiment, the resulting modified oligonucleotides of the present invention comprise a sequence where at least 80% of all purine nucleotides are 2′-deoxy purines and at least 80% of all pyrimidine nucleotides are 2′-O-methylpyrimidines. In a more preferred embodiment, the resulting modified oligonucleotides of the present invention comprise a sequence where at least 90% of all purine nucleotides are 2′-deoxy purines and at least 90% of all pyrimidine nucleotides are 2′-O-methylpyrimidines. In a most preferred embodiment, the resulting modified oligonucleotides of the present invention comprise a sequence where 100% of all purine nucleotides are 2′-deoxy purines and 100% of all pyrimidine nucleotides are 2′-methylpyrimidines.

Under rGmH transcription conditions of the present invention, the transcription reaction mixture comprises 2′-OH guanosine triphosphates, 2′-O-methyl cytidine triphosphates, 2′-O-methyl uridine triphosphates, and 2′-O-methyl adenosine triphosphates. The modified oligonucleotides produced using the rGmH transcription mixtures of the present invention comprise substantially all 2′-OH guanosine, 2′-O-methyl cytidine, 2′-O-methyl uridine, and 2′-O-methyl adenosine. In a preferred embodiment, the resulting modified oligonucleotides comprise a sequence where at least 80% of all guanosine nucleotides are 2′-OH guanosine, at least 80% of all cytidine nucleotides are 2′-O-methyl cytidine, at least 80% of all uridine nucleotides are 2′-O-methyl uridine, and at least 80% of all adenosine nucleotides are 2′-O-methyl adenosine. In a more preferred embodiment, the resulting modified oligonucleotides comprise a sequence where at least 90% of all guanosine nucleotides are 2′-OH guanosine, at least 90% of all cytidine nucleotides are 2′-O-methyl cytidine, at least 90% of all uridine nucleotides are 2′-O-methyl uridine, and at least 90% of all adenosine nucleotides are 2′-O-methyl adenosine. In a most preferred embodiment, the resulting modified oligonucleotides comprise a sequence where 100% of all guanosine nucleotides are 2′-OH guanosine, 100% of all cytidine nucleotides are 2′-O-methyl cytidine, 100% of all uridine nucleotides are 2′-O-methyl uridine, and 100% of all adenosine nucleotides are 2′-O-methyl adenosine.

Under r/mGmH transcription conditions of the present invention, the transcription reaction mixture comprises 2′-O-methyl adenosine triphosphate, 2′-O-methyl cytidine triphosphate, 2′-O-methyl guanosine triphosphate, 2′-O-methyl uridine triphosphate and deoxy guanosine triphosphate. The resulting modified oligonucleotides produced using the r/mGmH transcription mixtures of the present invention comprise substantially all 2′-O-methyl adenosine, 2′-O-methyl cytidine, 2′-O-methyl guanosine, and 2′-O-methyl uridine, wherein the population of guanosine nucleotides has a maximum of about 10% deoxy guanosine. In a preferred embodiment, the resulting r/mGmH modified oligonucleotides of the present invention comprise a sequence where at least 80% of all adenosine nucleotides are 2′-O-methyl adenosine, at least 80% of all cytidine nucleotides are 2′-O-methyl cytidine, at least 80% of all guanosine nucleotides are 2′-O-methyl guanosine, at least 80% of all uridine nucleotides are 2′-O-methyl uridine, and no more than about 10% of all guanosine nucleotides are deoxy guanosine. In a more preferred embodiment, the resulting modified oligonucleotides comprise a sequence where at least 90% of all adenosine nucleotides are 2′-O-methyl adenosine, at least 90% of all cytidine nucleotides are 2′-O-methyl cytidine, at least 90% of all guanosine nucleotides are 2′-O-methyl guanosine, at least 90% of all uridine nucleotides are 2′-O-methyl uridine, and no more than about 10% of all guanosine nucleotides are deoxy guanosine. In a most preferred embodiment, the resulting modified oligonucleotides comprise a sequence where 100% of all adenosine nucleotides are 2′-O-methyl adenosine, 100% of all cytidine nucleotides are 2′-O-methyl cytidine, 90% of all guanosine nucleotides are 2′-O-methyl guanosine, and 100% of all uridine nucleotides are 2′-O-methyl uridine, and no more than about 10% of all guanosine nucleotides are deoxy guanosine.

Under fGmH transcription conditions of the present invention, the transcription reaction mixture comprises 2′-O-methyl adenosine triphosphates (ATP), 2′-O-methyl uridine triphosphates (UTP), 2′-O-methyl cytidine triphosphates (CTP), and 2′-F guanosine triphosphates. The modified oligonucleotides produced using the fGmH transcription conditions of the present invention comprise substantially all 2′-O-methyl adenosine, 2′-O-methyl uridine, 2′-O-methyl cytidine, and 2′-F guanosine. In a preferred embodiment, the resulting modified oligonucleotides comprise a sequence where at least 80% of all adenosine nucleotides are 2-O-methyl adenosine, at least 80% of all uridine nucleotides are 2′-O-methyl uridine, at least 80% of all cytidine nucleotides are 2′-O-methyl cytidine, and at least 80% of all guanosine nucleotides are 2′-F guanosine. In a more preferred embodiment, the resulting modified oligonucleotides comprise a sequence where at least 90% of all adenosine nucleotides are 2′-O-methyl adenosine, at least 90% of all uridine nucleotides are 2′-O-methyl uridine, at least 90% of all cytidine nucleotides are 2′-O-methyl cytidine, and at least 90% of all guanosine nucleotides are 2′-F guanosine. The resulting modified oligonucleotides comprise a sequence where 100% of all adenosine nucleotides are 2′-O-methyl adenosine, 100% of all uridine nucleotides are 2′-O-methyl uridine, 100% of all cytidine nucleotides are 2′-O-methyl cytidine, and 100% of all guanosine nucleotides are 2′-F guanosine.

Under dAmB transcription conditions of the present invention, the transcription reaction mixture comprises 2′-deoxy adenosine triphosphates (dATP), 2′-O-methyl cytidine triphosphates (CTP), 2′-O-methyl guanosine triphosphates (GTP), and 2′-O-methyl uridine triphosphates (UTP). The modified oligonucleotides produced using the dAmB transcription mixtures of the present invention comprise substantially all 2′-deoxy adenosine, 2′-O-methyl cytidine, 2′-O-methyl guanosine, and 2′-O-methyl uridine. In a preferred embodiment, the resulting modified oligonucleotides comprise a sequence where at least 80% of all adenosine nucleotides are 2′-deoxy adenosine, at least 80% of all cytidine nucleotides are 2′-O-methyl cytidine, at least 80% of all guanosine nucleotides are 2′-O-methyl guanosine, and at least 80% of all uridine nucleotides are 2′-O-methyl uridine. In a more preferred embodiment, the resulting modified oligonucleotides comprise a sequence where at least 90% of all adenosine nucleotides are 2′-deoxy adenosine, at least 90% of all cytidine nucleotides are 2′-O-methyl cytidine, at least 90% of all guanosine nucleotides are 2′-O-methyl guanosine, and at least 90% of all uridine nucleotides are 2′-O-methyl uridine. In a most preferred embodiment, the resulting modified oligonucleotides of the present invention comprise a sequence where 100% of all adenosine nucleotides are 2′-deoxy adenosine, 100% of all cytidine nucleotides are 2′-O-methyl cytidine, 100% of all guanosine nucleotides are 2′-O-methyl guanosine, and 100% of all uridine nucleotides are 2′-O-methyl uridine.

In each case, the transcription products can then be used as the library in the SELEX™ process to identify aptamers and/or to determine a conserved motif of sequences that have binding specificity to a given target. The resulting sequences are already stabilized, eliminating this step from the process to arrive at a stabilized aptamer sequence and giving a more highly stabilized aptamer as a result. Another advantage of the 2′-OMe SELEX™ process is that the resulting sequences are likely to have fewer 2′-OH nucleotides required in the sequence, possibly none.

As described below, lower but still useful yields of transcripts fully incorporating 2′-OMe substituted nucleotides can be obtained under conditions other than the optimized conditions described above. For example, variations to the above transcription conditions include:

The HEPES buffer concentration can range from 0 to 1 M. The present invention also contemplates the use of other buffering agents having a pKa between 5 and 10, for example without limitation, Tris(hydroxymethyl)aminomethane.

The DTT concentration can range from 0 to 400 mM. The methods of the present invention also provide for the use of other reducing agents, for example without limitation, mercaptoethanol.

The spermidine and/or spermine concentration can range from 0 to 20 mM.

The PEG-8000 concentration can range from 0 to 50% (w/v). The methods of the present invention also provide for the use of other hydrophilic polymer, for example without limitation, other molecular weight PEG or other polyalkylene glycols.

The Triton X-100 concentration can range from 0 to 0.1% (w/v). The methods of the present invention also provide for the use of other non-ionic detergents, for example without limitation, other detergents, including other Triton-X detergents.

The MgCl₂ concentration can range from 0.5 mM to 50 mM. The MnCl₂ concentration can range from 0.15 mM to 15 mM. Both MgCl₂ and MnCl₂ must be present within the ranges described and in a preferred embodiment are present in about a 10 to about 3 ratio of MgCl₂:MnCl₂, preferably, the ratio is about 3-5, more preferably, the ratio is about 3 to about 4.

The 2′-OMe NTP concentration (each NTP) can range from 5 μM to 5 mM.

The 2′-OH GTP concentration can range from 0 μM to 300 μM.

The 2′-OH GMP concentration can range from 0 to 5 mM.

The pH can range from pH 6 to pH 9. The methods of the present invention can be practiced within the pH range of activity of most polymerases that incorporate modified nucleotides.

In addition, the methods of the present invention provide for the optional use of chelating agents in the transcription reaction condition, for example without limitation, EDTA, EGTA, and DTT.

Pharmaceutical Compositions

The invention also includes pharmaceutical compositions containing the aptamer molecules described herein. In some embodiments, the compositions are suitable for internal use and include an effective amount of a pharmacologically active compound of the invention, alone or in combination, with one or more pharmaceutically acceptable carriers. The compounds are especially useful in that they have very low, if any toxicity.

Compositions of the invention can be used to treat or prevent a pathology, such as a disease or disorder, or alleviate the symptoms of such disease or disorder in a patient. Compositions of the invention are useful for administration to a subject suffering from, or predisposed to, a disease or disorder which is related to or derived from a target to which the aptamers specifically bind.

For example, the target is a protein involved with a pathology, for example, the target protein causes the pathology.

Compositions of the invention can be used in a method for treating a patient having a pathology. The method involves administering to the patient a composition comprising aptamers that bind a target (e.g., a protein) involved with the pathology, so that binding of the composition to the target alters the biological function of the target, thereby treating the pathology.

The patient having a pathology, e.g. the patient treated by the methods of this invention can be a mammal, or more particularly, a human.

In practice, the compounds or their pharmaceutically acceptable salts, are administered in amounts which will be sufficient to exert their desired biological activity.

For instance, for oral administration in the form of a tablet or capsule (e.g., a gelatin capsule), the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol and the like. Disintegrators include, without limitation, starch, methyl cellulose, agar, bentonite, xanthan gum starches, agar, alginic acid or its sodium salt, or effervescent mixtures, and the like. Diluents, include, e.g., lactose, dextrose, sucrose, mannitol, sorbitol, cellulose and/or glycine.

Injectable compositions are preferably aqueous isotonic solutions or suspensions, and suppositories are advantageously prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating or coating methods, respectively, and contain about 0.1 to 75%, preferably about 1 to 50%, of the active ingredient.

The compounds of the invention can also be administered in such oral dosage forms as timed release and sustained release tablets or capsules, pills, powders, granules, elixirs, tinctures, suspensions, syrups and emulsions.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the injectable solution or suspension. Additionally, solid forms suitable for dissolving in liquid prior to injection can be formulated. Injectable compositions are preferably aqueous isotonic solutions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances.

The compounds of the present invention can be administered in intravenous (both bolus and infusion), intraperitoneal, subcutaneous or intramuscular form, all using forms well known to those of ordinary skill in the pharmaceutical arts. Injectables can be prepared in conventional forms, either as liquid solutions or suspensions.

Parental injectable administration is generally used for subcutaneous, intramuscular or intravenous injections and infusions. Additionally, one approach for parenteral administration employs the implantation of a slow-release or sustained-released systems, which assures that a constant level of dosage is maintained, according to U.S. Pat. No. 3,710,795, incorporated herein by reference.

Furthermore, preferred compounds for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels, wherein the concentration of active ingredient would range from 0.01% to 15%, w/w or w/v.

For solid compositions, excipients include pharmaceutical grades of mannitol, lactose, starch, magnesium stearate, sodium saccharin, talcum, cellulose, glucose, sucrose, magnesium carbonate, and the like may be used. The active compound defined above, may be also formulated as suppositories using for example, polyalkylene glycols, for example, propylene glycol, as the carrier. In some embodiments, suppositories are advantageously prepared from fatty emulsions or suspensions.

The compounds of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564. For example, the aptamer molecules described herein can be provided as a complex with a lipophilic compound or non-immunogenic, high molecular weight compound constructed using methods known in the art. An example of nucleic-acid associated complexes is provided in U.S. Pat. No. 6,011,020.

The compounds of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

If desired, the pharmaceutical composition to be administered may also contain minor amounts of non-toxic auxiliary substances such as wetting or emulsifying agents, pH buffering agents, and other substances such as for example, sodium acetate, triethanolamine, oleate, etc.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

Oral dosages of the present invention, when used for the indicated effects, will range between about 0.05 to 1000 mg/day orally. The compositions are preferably provided in the form of scored tablets containing 0.5, 1.0, 2.5, 5.0, 10.0, 15.0, 25.0, 50.0, 100.0, 250.0, 500.0 and 1000.0 mg of active ingredient. Effective plasma levels of the compounds of the present invention range from 0.002 mg to 50 mg per kg of body weight per day.

Compounds of the present invention may be administered in a single daily dose, or the total daily dosage may be administered in divided doses of two, three or four times daily.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same.

The invention having now been described by way of written description, those of skill in the art will recognize that the invention can be practiced in a variety of embodiments and that the foregoing description and examples below are for purposes of illustration and not limitation of the claims that follow.

### EXAMPLES

### Example 1

### 2′-OMe SELEX™ Against Thrombin and VEGF Targets

A library of approximately 3×10¹⁴ unique transcription templates, each containing a random region of thirty contiguous nucleotides, was synthesized as described below, and PCR amplified. Cloning and sequencing of this library demonstrated that the composition of the random region in this library was approximately 25% of each nucleotide. The DNA library was purified away from unincorporated dNTPs by gel-filtration and ethanol-precipitation. Modified transcripts were then generated from a mixture containing 500 uM of each of the four 2′-OMe NTPs, i.e., A, C, U and G, and 30 uM 2′-OH GTP (“r/mGmH”). In addition, modified transcripts were generated from mixtures containing part modified nucleotides and part ribonucleotides or all ribonucleotides namely, a mixture containing all 2′-OH nucleotides (rN); a mixture containing 2′-OMe C and U and 2′-OH A and G (rRmY); a mixture containing 2′-OMe A, C, and U, and 2′-OH G (“rGmH”); and a mixture alternately containing 2′-OMe A, C, U and G and 2′-OMe A, U and C and 2′-F G (“toggle”). These modified transcripts were then used in SELEX™ against targets—e.g., VEGF and thrombin.

Generally, after gel-purification and DNase-treatment these modified transcripts were dissolved in PBS for VEGF or 1×ASB (150 mM KCl, 20 mM HEPES, 10 mM MgCl₂, 1 mM DTT, 0.05% Tween20, pH 7.4) for thrombin, and incubated for one hour in an empty well on a hydrophobic multiwell plate to subtract plastic-binding sequences. The supernatant was then transferred to a well that had previously been incubated for one hour at room temperature in PBS for VEGF or in ASBND (150 mM KCl, 20 mM HEPES, 10 mM MgCl₂, 1 mM DTT, pH 7.4) for thrombin. After a one hour incubation the well was washed and bound sequences were reverse-transcribed in situ using thermoscript reverse transcriptase (Invitrogen) at 65° C. for one hour. The resultant cDNA was then PCR-amplified, separated from dNTPs by gel-filtration, and used to generate modified transcripts for input into the next round of selection. After 10 rounds of selection and amplification the ability of the resultant library to bind to VEGF or thrombin was assessed by Dot-Blot. At this point, the library was cloned, sequenced and individual clones were assayed for their ability to bind VEGF or thrombin. Using this combination of sequence and clonal binding data, sequence motifs were identified.

One VEGF aptamer motif, exemplified by ARC224, which was common to both the r/mGmH and toggle selections, was used to design smaller synthetic constructs which were also assayed for binding to VEGF and ultimately minimized aptamers to VEGF were identified, ARC245 and ARC259, both of which are 23 nucleotides long. Another VEGF aptamer motif, exemplified by ARC226, which was common to all 2′-OMe selections, was also identified. The ARC224 aptamer produced by the methods of the present invention has the sequence 5′-mCmGmAmUmAmUmGmCmAmGmUmUmUmGmAmGmAmAmGmUmCmGmCmGmC mAmUmUmCmG-3T (SEQ ID No. 184) where “m” represents a 2′-O-methyl substitution.

The ARC226 aptamer has the sequence:

| | |
|---|---|
| 5-mGmAmUmCmAmUmGmCmAmUGmUmGmGmAmUm | (SEQ ID No. 186) |
| CmGmCmGmGmAmUmC-[3T]-3′. | |

The ARC245 aptamer has sequence:

| | |
|---|---|
| 5′-mAmUmGmCmAmGmUmUmUmGmAmGmAmAmGm | (SEQ ID No. 187) |
| UmCmGmCmGmCmAmU-[3T]-3′. | |

The ARC259 aptamer has the sequence:

| | |
|---|---|
| 5′-mAmCmGmCmAmGmUmUmUmGmAmGmAmAmGm | (SEQ ID No. 188) |
| UmCmGmCmGmCmGMu-[3T]-3′. | |

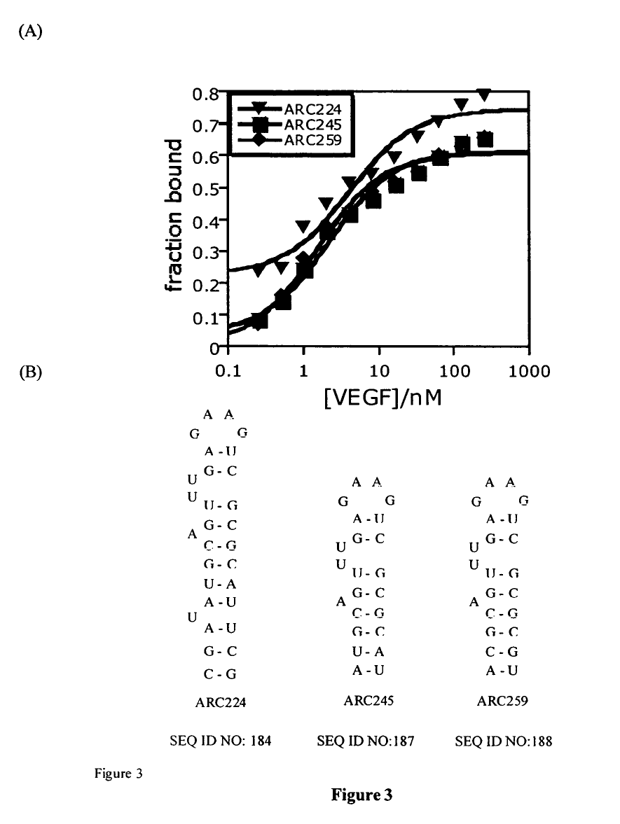

FIG. 3A is a graph of VEGF binding by ARC224, ARC245 and ARC259. A schematic representation of the secondary structure of these aptamers is presented in FIG. 3B.

All residues in ARC224, ARC226 and ARC245 are 2′-OMe and all constructs (initially identified by SELEX™) were generated by solid-phase chemical synthesis. The K_{D} values of these aptamers, determined by dot-blot in PBS, are as follows: ARC224 3.9 nM, ARC245 2.1 nM, ARC259 1.4 nM.

Reagents. All reagents were acquired from Sigma (St. Louis, Mo.) except where otherwise stated.

Oligonucleotide synthesis. DNA syntheses were undertaken according to standard protocols using an Expedite 8909 DNA synthesizer (Applied Biosystems, Foster City, Calif.). The DNA library used in this study had the following sequence: ARC254: 5′-CATCGATGCTAGTCGTAACGATCNCGAGAACGTTCTCTCCTCTCCCTATAGTGAGTCGTATTA-3′ (SEQ ID NO:1) in which each N has an equal probability of being each of the four nucleotides. 2′-OMe RNA syntheses, including those containing 2′-OH nucleotides, were undertaken according to standard protocols using a 3900 DNA Synthesizer (Applied Biosystems, Foster City, Calif.). All oligonucleotides were purified by denaturing PAGE except PCR and RT primers.

2′-OMe Library Generation. The synthetic DNA library (1.5 mmol) was amplified by PCR under standard conditions with the following primers:

| | |
|---|---|
| 3′-primer | |
| 5′-CATCGATGCTAGTCGTAACGATCC-3′ | (SEQ ID NO:454) |
| and | |
| 5′-primer | |
| 5′-TAATACGACTCACTATAGGGAGAGGAGAGAA | (SEQ ID NO:455) |
| ACGTTCTCG-3′. | |

The resultant library of double-stranded transcription templates was precipitated and separated from unincorporated nucleotides by gel-filtration. At no point was the library denatured, either by thermal means or by exposure to low-salt conditions. r/mGmH transcription was performed under the following conditions to produce template for the first round of selection: double-stranded DNA template 200 nM, HEPES 200 mM, DTT 40 mM, Triton X-100 0.01%, Spermidine 2 mM, 2′-O-methyl ATP, CTP, GTP and UTP 500 μM each, 2′-OH GTP 30 uM, GMP 500 μM, MgCl₂ 5.0 mM, MnCl₂ 1.5 mM, inorganic pyrophosphatase 0.5 units per 100 μL reaction, Y639F/H784A T7 RNA polymerase 1.5 units per 100 μl reaction pH 7.5 and 10% w/v PEG and were incubated at 37° C. overnight. The resultant transcripts were purified by denaturing 10% PAGE, eluted from the gel, incubated with RQ1 DNase (Promega, Madison Wis.), phenol-extracted, chloroform-extracted, precipitated and taken up in PBS. For the initiation of selection transcripts were additionally generated by the direct chemical synthesis of 2′-OMe RNA, these were purified by denaturing 10% polyacrylamide gel electrophoresis, eluted from the gel and taken up in PBS.

For the rN, rRmY and rGmH transcriptions, the transcription conditions were as follows, where 1×Tc buffer is: 200 mM HEPES, 40 mM DTT, 2 mM Spermidine, 0.01% Triton X-100, pH 7.5.

When 2′-OH A, C, U and G (rN) conditions were used, the transcription reaction conditions were MgCl₂ 25 mM, each NTP 5 mM, 1×Tc buffer, 10% w/v PEG, T7 RNA polymerase 1.5 units, and 50-200 nM double stranded template (200 nM of template was used in Round 1 to increase diversity and for subsequent rounds approximately 50 nM, a 1/10 dilution of an optimized PCR reaction using conditions described herein, was used).

When 2′-OMe C and U and 2′-OH A and G (rRmY) conditions were used, the transcription reaction conditions were 1×Tc buffer, 50-200 nM double stranded template (200 nM of template was used in Round 1 to increase diversity and for subsequent rounds approximately 50 nM, a 1/10 dilution of an optimized PCR reaction using conditions described herein, was used), 5.0 mM MgCl₂, 1.5 mM MnCl₂, 0.5 mM each base, 10% PEG-8000, 0.25 units inorganic pyrophosphatase, and 1.5 units Y639F/H784A T7 RNA polymerase.

When 2′-OMe A, C, and U and 2′-OH G (rGmH) conditions were used, the transcription reaction conditions were 1×Tc buffer, 50-200 nM double stranded DNA template (200 nM of template was used in Round 1 to increase diversity for subsequent rounds approximately 50 nM, a 1/10 dilution of an optimized PCR reaction using conditions described herein, was used), 5.0 mM MgCl₂, 1.5 mM MnCl₂, 0.5 mM each base, 10% PEG-8000, 0.25 units inorganic pyrophosphatase, and 1.5 units Y639F single mutant T7 RNA polymerase in 100 μl volume.

When 2′-OMe A, C, U and 2′-F G conditions were used, the transcription reaction conditions were as for rGmH, except 0.5 mM 2′-F GTP is used instead of 2′-OH GTP.

Reverse Transcription. The reverse transcription conditions used during SELEX™ are as follows (100 μL reaction volume): 1× Thermo buffer (Invitrogen), 4 μM primer, 10 mM DTT, 0.2 mM each dNTP, 200 μM Vanadate nucleotide inhibitor, 10 μg/ml tRNA, Thermoscript RT enzyme 1.5 units (Invitrogen). Reverse transcriptase reaction yields are lower for 2′-OMe templates. PCR reaction conditions are as follows 1× ThermoPol buffer (NEB), 0.5 μM 5′ primer, 0.5 μM 3′ primer 0.2 mM each DHTP, Taq DNA Polymerase 5 units (NEB).

2′-OMe SELEX™ Protocol. As noted above, SELEX™ was performed with the modified transcripts against each of two targets (VEGF and Thrombin) using 5 kinds of transcripts for a total of 10 selections. The five kinds of transcripts were: “rN” (all 2′-OH), “rRmY” (2′-OH A, G, 2′-OMe C, U), “rGmH” (2′-OH G, 2′-OMe C, U, A), “r/mGmH” (2′-OMe A, U, G, C 500 uM, 2′-OH G 30 uM), “toggle” (alternately “r/mGmH” and 2′-OMe A, U, C, 2′-F G).

All of the selections directed against VEGF generated VEGF specific aptamers while only the rN and rRmY selections against thrombin generated thrombin specific aptamers. The aptamer sequences identified in these selections are set forth in Tables 1 through 5 (VEGF) and Tables 6 through 10 (thrombin) below.

The sequences are from SELEX™ round 11 except for Thrombin “rGmH”, “r/mGmH” and “toggle” which are from round 5, VEGF “r/mGmH” which is from round 10 and VEGF “toggle” which is from round 8.

The selection was performed by initially immobilizing the protein by hydrophobic absorption to “NUNC MAXY” plates, washing away the protein that didn't bind, incubating the library of 2′-OMe-substituted transcripts with the immobilized protein, washing away the transcripts that didn't bind, performing RT directly in the plate, then PCR, and then transcribing the resultant double-stranded DNA template under the appropriate transcription conditions.

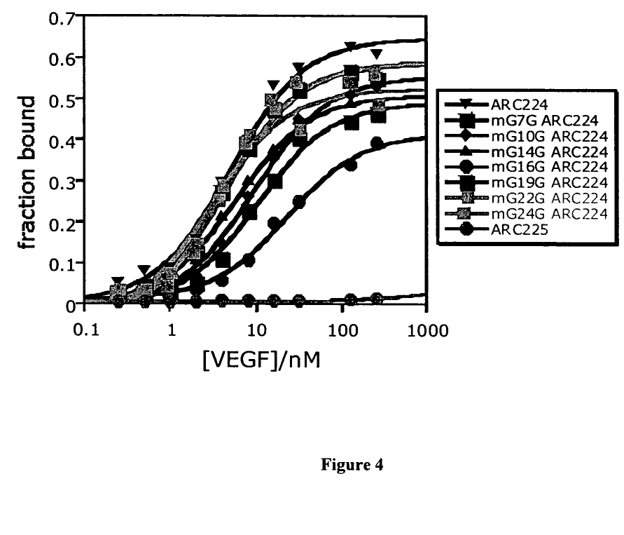

Binding assays were performed with trace ³²P-body-labelled transcripts that were incubated with various protein concentrations in silanized wells, these were then passed through a sandwich of a nitrocellulose membrane over a nylon membrane. Protein-bound RNA is visualized on the NC membrane, unbound RNA on the nylon membrane. The proportion binding is then used to calculate affinity (see FIGS. 4, 5, and 6). For example, the binding characteristics of various 2′-OH G variants of ARC224 (all 2-OMe) are shown in FIG. 4. The nomenclature “mGXG” indicates a substitution of 2′-OH G for 2′-OMe G at position “X”, as numbered sequentially from the 5′-terminus. Thus, mG7G ARC224 is ARC224 with a 2′-OH at position 7. ARC225 is ARC224 with 2′-OMe to 2′-OH substitutions at positions 7, 10, 14, 16, 19, 22 and 24. All constructs (initially identified by SELEX™) were generated by solid-phase chemical synthesis. These data were generated by dot-blot in PBS. The fully 2′-OMe aptamer, ARC224, has superior VEGF-binding characteristics when compared to any of the 2′-OH substituted variants studied.

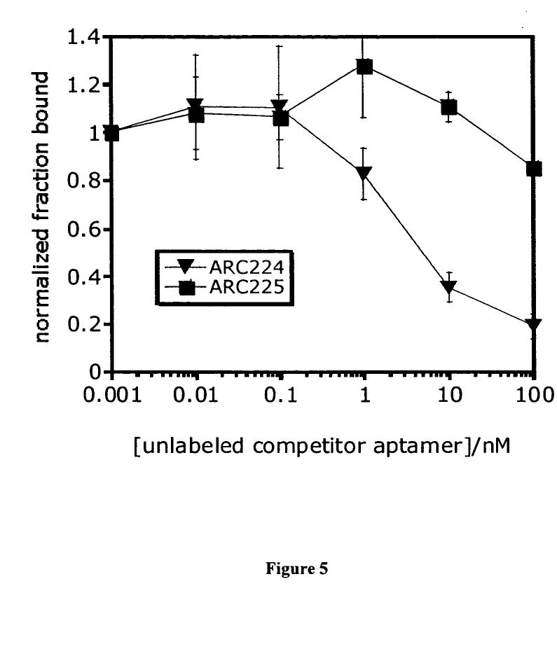

FIG. 5 is a plot of ARC224 and ARC225 binding to VEGF. This graph indicates that ARC224 binds VEGF in a manner which inhibits the biological function of VEGF. ¹²I-labeled VEGF was incubated with the aptamer and this mixture was then incubated with human umbilical cord vascular endothelial cells (HUVEC). The supernatant was removed, the cells were washed, and bound VEGF was counted in a scintillation counter. ARC225 has the same sequence as ARC224 and 2′-OMe to 2′-OH substitutions at positions 7, 10, 14, 16, 19, 22 and 24 numbered from the 5′-terminus. These data indicate that the IC₅₀ of ARC224 is approximately 2 nM.

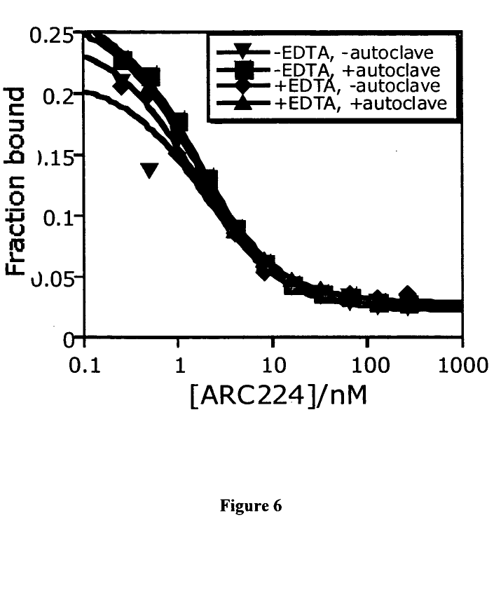

FIG. 6 is a binding curve plot of ARC224 binding to VEGF before and after autoclaving, with or without EDTA. FIG. 6 shows both the proportion of aptamer that is functional and the IC₅₀ for binding to VEGF before and after autoclaving for 25 minutes with a peak temperature of 125° C. These data were determined by the inhibition by unlabeled ARC224 of the binding of 5′-labeled ARC224 to 1 nM VEGF in PBS as measured by dot-blot in PBS. Where indicated, samples contained 1 mM EDTA. All constructs (initially identified by SELEX™) were generated by solid-phase chemical synthesis. No degradation of ARC224 was observed within the limitations of this assay.

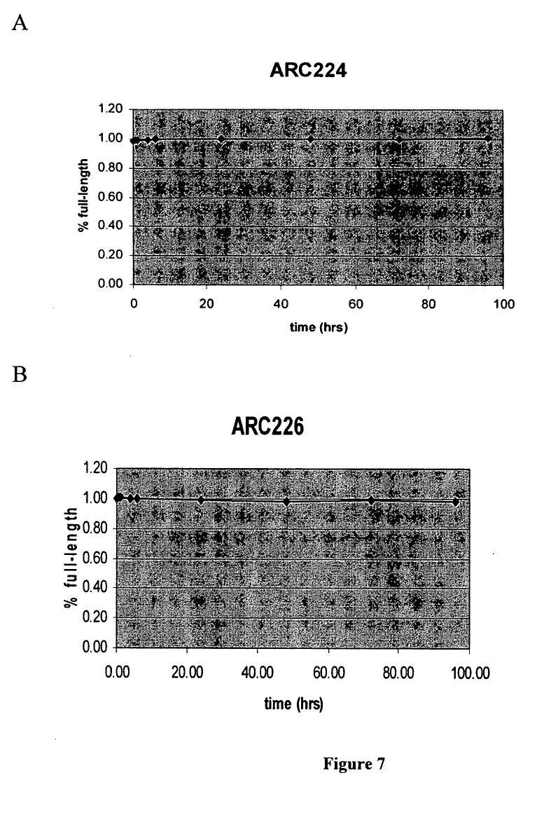

Degradation studies show that incubation in plasma at 37° C. over 4 days induces so little degradation that measuring a half-life is not possible, but is at least in excess of 4 days (see, e.g., FIG. 7). FIGS. 7A and 7B are plots of the stability of ARC224 and ARC226, respectively, when incubated at 37° C. in rat plasma. As indicated in the figure, both ARC224 and ACR226 showed no detectable degradation after for 4 days in rat plasma. In these experiments, 5′-labeled ARC224 and ARC226 were incubated in rat plasma at 37° C. and analyzed by denaturing PAGE. All constructs (initially identified by SELEX™) were generated by solid-phase chemical synthesis. The half-life appears to be in excess of 100 hours.

Tables 1 through Table 10 below show the DNA sequences of aptamers corresponding to the transcribed aptamers isolated from the various libraries, i.e. rN, rRmY, rGmH, and r/mGmH, as indicated. The sequence of the aptamers will have uridine residues instead of thymidine residues in the DNA sequences shown. Table 11 shows the stabilized aptamer sequences obtained by the methods of the present invention. As used herein, “3T” refers to an inverted thymidine nucleotide attached to the oligonucleotide phosphodiester backbone at the 5′ position, the resulting oligo having two 5′-OH ends and is thus resistant to 3′ nucleases.

Unless noted otherwise, individual sequences listed in the various tables represent the cDNA clones of the aptamers that were selected under the SELEX conditions provided. The actual aptamers provided in the invention are those corresponding sequences comprising the rN, mN, rRmY, rGmH, r/mGmH, dRmY and toggle combinations of residues, as indicated in the text.

2′-OMe SELEX™ Results.

TABLE 1

Corresponding cDNAs of the VEGF Aptamer
Sequences - all 2′-OH (rN)

SEQ ID No. 3 >PB.97.126.F_43-H1
GGGAGAGGAGAGAACGTTCTCGAAATGATGCATGTTCGTAAAATGGCAGT
ATTGGATCGTTACAACTAGCATCGATG
SEQ ID No. 4 >PB.97.126.F_43-A2
GGGAGAGGAGAGAACGTTCTCGTGCCGAGGTCCGGAACCTTGATGATTGG
CGGGATCGTTACGACTAGCATCGATG
SEQ ID No. 5 >PB.97.126.F_48-A1
GGGAGAGGAGAGAACGTTCTCGCATTTGGGCTAGTTGTGAAATGGCAGTA
TTGGATCGTTACGACTAGCATCGATG
SEQ ID No. 6 >PB.97.126.F_48-B1
GGGAGAGGAGAGAACGTTCTCGAATCGTAGATAGTCGTGAAATGGCAGTA
TTGGATCGTTACGACTAGCATCGATG
SEQ ID No. 7 >PB.97.126.F_48-C1
GGGAGAGGAGAGAACGTTCTCGTTCTAGTCGGTACGATATGTTGACGAAT
CCGGATCGTTACGACTAGCATCGATG
SEQ ID No. 8 >PB.97.126.F_48-D1
GGGAGAGGAGAGAACGTTCTCGTTTGATGAGGCGGACATAATCCGTGCCG
AGCGGGATCGTTACGACTAGCATCGATG
SEQ ID No. 9 >PB.97.126.F_48-E1
GGGAGAGGAGAGAACGTTCTCGAAGGAAAAGAGTTTAGTATTGGCCGTCC
GTGGGATCGTTACGACTAGCATCGATG
SEQ ID No. 10 >PB.97.126.F_48-F1
GGGAGAGGAGAGAACGTTCTCGTGCCGAGGTCCGGAACCTTGATGATTGG
CGGGATCGTTACGACTAGCATCGATG
SEQ ID No. 11 >PB.97.126.F_48-G1
GGGAGAGGAGAGAACGTTCTCGTACGGTCCATTGAGTTTGAGATGTCGCC
ATGGATCGTTACGACTAGCATCGATG
SEQ ID No. 12 >PB.97.126.F_48-B2
GGGAGAGGAGAGAACGTTCTCGAGTTAGTGGTAACTGATATGTTGAATTG
TCCGGATCGTTACGACTAGCATCGATG
SEQ ID No. 13 >PB.97.126.F_48-C2
GGGAGAGGAGAGAACGTTCTCGCACGGATGGCGAGAACAGAGATTGCTAG
GTGGATCGTTACGACTAGCATCGATG
SEQ ID No. 14 >PB.97.126.F_48-D2
GGGAGAGGAGAGAACGTTCTCGNTANCGNTNCGCCNTGCTAACGCNTANT
TGGGATCGTTACGACTAGCATCGATG
SEQ ID No. 15 >PB.97.126.F_48-E2
GGGAGAGGAGAGAACGTTCTCGAAGATGAGTTTTGTCGTGAAATGGCAGT
ATTGGATCGTTACGACTAGCATCGATG
SEQ ID No. 16 >PB.97.126.F_48-F2
GGGAGAGGAGAGAACGTTCTCGGGATGCCGGATTGATTTCTGATGGGTAC
TGGGATCGTTACGACTAGCATCGATG
SEQ ID No. 17 >PB.97.126.F_48-G2
GGGAGAGGAGAGAACGTTCTCGAATGGAATGCATGTCCATCGCTAGCATT
TGGGATCGTTACGACTAGCATCGATG
SEQ ID No. 18 >PB.97.126.F_48-H2
GGGAGAGGAGAGAACGTTCTCGTGCTGAGGTCCGGAACCTTGATGATTGG
CGGGATCGTTNCNACTAGCATCGATG
SEQ ID No. 19 >PB.97.126.F_48-A3
GGGAGAGGAGAGAACGTTCTCGCTAATTGCTGAGTCGTGAAGTGGCAGTA
TTGGATCGTTACGACTAGCATCGATG
SEQ ID No. 20 >PB.97.126.F_48-B3
GGGAGAGGAGAGAACGTTCTCGTAACGATGTCCGGGGCGAAAGGCTAGCA
TGGGATCGTTACGACTAGCATCGATG
SEQ ID No. 21 >PB.97.126.F_48-C3
GGGAGAGGAGAGAACGTTCTCGATGCGATTGTCGAGATTTGTAAGATAGC
TGTGGATCGTTACGACTAGCATCGATG

TABLE 2

Corresponding cDNAs of the VEGF Aptamer
Sequences - 2′-OH AG, 2′-OMe CU (rRmY)

SEQ ID No. 22 >PB.97.126.G_43-D3
GGGAGAGGAGAGAACGTTCTCGCAGAAAACATCTTTGCGGTTGAATACAT
GTGGATCGTTACGACTAGCATCGATG
SEQ ID No. 23 >PB.97.126.G_43-G3
GGGAGAGGAGAGAACGTTCTCGAAAAAAGANANCNNCCTTCNGAATACAT
GCGGATCGTTACGACTAGCATCGATG
SEQ ID No. 24 >PB.97.126.G_48-E3
GGGAGAGGAGAGAACGTTCTCGAGAGTGATTCGATGCTTCANGAATACAT
GTGGATCGTTACGACTAGCATCGATG
SEQ ID No. 25 >PB.97.126.G_48-F3
GGGAGAGGAGAGAACGTTCTCGAGAGTGATTCGATGCTTCANGAATACAT
GTGGATCGTTACGACTAGCATCGATG
SEQ ID No. 26 >PB.97.126.G_48-H3
GGGAGAGGAGAGAACGTTCTCGAAGAAGGAAAGCTGCAAGTCGAATACAC
GCGGATCGTTACGACTAGCATCGATG
SEQ ID No. 27 >PB.97.126.G_48-A4
GGGAGAGGAGAGAACGTTCTCGCAAAAACATCGATTACAGTTGAGTACAT
GTGGATCGTTACGACTAGCATCGATG
SEQ ID No. 28 >PB.97.126.G_48-B4
GGGAGAGGAGAGAACGTTCTCGAGACATCATTGCTCGTTGAATACATGTG
GATCGTTACGACTAGCATCGATG
SEQ ID No. 29 >PB.97.126.G_48-C4
GGGAGAGGAGAGAACGTTCTCGCCAAAGTAGCTTCGACAGTCGAATACAT
GTGGATCGTTACGACTAGCATCGATG
SEQ ID No. 30 >PB.97.126.G_48-D4
GGGAGAGGAGAGAACGTTCTCGAAAATCAGTACTGTGCAGTCGAATACAT
GCGGATCGTTACGACTAGCATCGATG
SEQ ID No. 31 >PB.97.126.G_48-E4
GGGAGAGGAGAGAACGTTCTCGTAATGACATCAATGCTTCTTGAATACAG
GTGGATCGTTACGACTAGCATCGATG
SEQ ID No. 32 >PB.97.126.G_48-F4
GGGAGAGGAGAGAACGTTCTCGAGAAAAACGATCTGTGACGTGTAATCCG
CGGATCGTTACGACTAGCATCGATG
SEQ ID No. 33 >PB.97.126.G_48-G4
GGGAGAGGAGAGAACGTTCTCGCAACAAACGTCGACGCTTCTGAATACAT
GTGGATCGTTACGACTAGCATCGATG
SEQ ID No. 34 >PB.97.126.G_48-H4
GGGAGAGGAGAGAACGTTCTCGTGATCATAGAAATGCTAGCTGAATACAT
GTGGATCGTTACGACTAGCATCGATG
SEQ ID No. 35 >PB.97.126.G_48-A5
GGGAGAGGAGAGAACGTTCTCGCAGCGTAAAATGCTTTTCGAAGTACATG
TGGATCGTTACGACTAGCATCGATG
SEQ ID No. 36 SEQ ID No. >PB.97.126.G_48-B5
GGGAGAGGAGAGAACGTTCTCGCCAAGAATCAATCGCTTGTCGAATACAT
GCGGATCGTTACGACTAGCATCGATG
SEQ ID No. 37 >PB.97.126.G_48-C5
GGGAGAGGAGAGAACGTTCTCGTGATCATAGAAATGCTAGCTGAGTACAT
GTGGATCGTTACGACTAGCATCGATG
SEQ ID No. 38 >PB.97.126.G_48-D5
GGGAGAGGAGAGAACGTTCTCGCAGAAAACATCTTTGCGGTTGAATACAT
GTGGATCGTTACGACTAGCATCGATG
SEQ ID No. 39 >PB.97.126.G_48-E5
GGGAGAGGAGAGAACGTTCTCGNAAACANNCATCTATTGNAGTTGAATAC
ATGTGGATCGTTACGACTAGCATCGATG
SEQ ID No. 40 >PB.97.126.G_48-F5
GGGAGAGGAGAGAACGTTCTCGCTAAAGATTCGCTGCTTGCCGAATACAT
GTGGATCGTTACGACTAGCATCGATG

TABLE 3

Corresponding cDNAs of the VEGF Aptamer
Sequences - 2′-OH G, 2′-OMe CUA (rGmH)

SEQ ID No. 41 >PB.97.126.H_43-H6
GGGAGAGGAGAGAACGTTCTCGGGTTTTGTCTGCGTTTGTGCGTTGAACC
CGGGATCGTTACGACTAGCATCGATG
SEQ ID No. 42 >PB.97.126.H_43-F7
GGGAGAGGAGAGAACGTTCTCGTGATTACGTGATGAGGATCCGCGTTTTC
TCGGGATCGTTACGACTAGCATCGATG
SEQ ID No. 43 >PB.97.126.H_43-H7
GGGAGAGGAGAGAACGTTCTCGTTAGTGAAAACGATCATGCATGTGGATC
GCGGATCGTTACGACTAGCATCGATG
SEQ ID No. 44 >PB.97.126.H_48-H5
GGGAGAGGAGAGAACGTTCTCGTGTTCATTCGTTTGCTTATCGTTGCATG
TGGATCGTTACGACTAGCATCGATG
SEQ ID No. 45 >PB.97.126.H_48-A6
AGGAGAGGAGAGAACGTTCTCGGCAGAGTGTGATGTGCATCCGCACGTGC
CGGGATCGTTACGACTAGCATCGATG
SEQ ID No. 46 >PB.97.126.H_48-B6
GGGAGAGGAGAGAACGTTCTCGTTAGTAAATACGATCGTGCATGTGGATC
GCGGATCGTTACGACTAGCATCGATG
SEQ ID No. 47 >PB.97.126.H_48-C6
GGGAGAGGAGAGAACGCCCCCCTGATTNCGTGAAGAGGATCCGCANTTTC
NCGGGATCGTTACGACTAGCATCGATG
SEQ ID No. 48 >PB.97.126.H_48-D6
GGGAGAGGAGAGAACGTTCTCGTGGCTTTGGAACGGGTACGGATTTGGCA
CGGGATCGTTACGACTAGCATCGATG
SEQ ID No. 49 >PB.97.126.H_48-E6
GGGAGAGGAGAGAACGTTCTCGTGATTACGTGATGAGGATCCGCGTTTTC
TCGGGATCGTTACGACTAGCATCGATG
SEQ ID No. 50 >PB.97.126.H_48-F6
GGGAGAGGAGAGAACGTTCTCGTCATTGGTGACNGCGTTGCATGTGGATC
GCGGATCGTTACGACTAGCATCGATG
SEQ ID No. 51 >PB.97.126.H_48-G6
GGGAGAGGAGAGAACGTTCTCGNTGGTNNAANGCTTTTGTNGGGNTANNT
GTGGATCGTTACGACTAGCATCGATG
SEQ ID No. 52 SEQ ID No. >PB.97.126.H_48-A7
GGGAGAGGAGAGAACGTTCTCGTGGCTTTGGAACGAATTCGGATTTGGCA
CGGGATCGTTACGACTAGCATCGATG
SEQ ID No. 53 >PB.97.126.H_48-B7
GGGAGAGGAGAGAACGTTCTCGTGCGATGTCGTGGATTTCCGTTTCGCAA
GGGATCGTTACGACTAGCATCGATG
SEQ ID No. 54 PB.97.126.H_48-C7
GGGAGAGGAGAGAACGTTCTCGTGAAGCAGATGTCGTTGGCGACTTAGAG
GGGGATCGTTACGACTAGCATCGATG
SEQ ID No. 55 >PB.97.126.H_48-D7
GGGAGAGGAGAGAACGTTCTCGTGATTTCGTGATGAGGATCCGCGTTTTC
TCGGGATCGTTACGACTAGCATCGATG
SEQ ID No. 56 >PB.97.126.H_48-E7
GGGAGAGGAGAGAACGTTCTCGCTAGTAACGATGACTTGATGAGCATCCG
AGGATCGTTACGACTAGCATCGATG
SEQ ID No. 57 >PB.97.126.H_48-G7
GGGAGAGGAGAGAACGTTCTCGTCATAAGTAACGACGTTGCATGTGGATC
GCGGATCGTTACGACTAGCATCGATG
SEQ ID No. 58 >PB.97.126.H_48-A8
GGGAGAGGAGAGAACGTTCTCGCAAGGAGATGGTTGCTAGCTGAGTACAT
GTGGATCGTTACGACTAGCATCGATG

TABLE 4

Corresponding cDNAs of the VEGF Aptamer
Sequences - 2′-OMe AUGC (r/mGmH, each G has a 90%
probability of having a 2′-OMe group
incorporated therein)

SEQ ID No. 59 PB.97.126.I_43-B8
GGGAGAGGAGAGAACGTTCTCGCGATATGCAGTTTGAGAAGTCGCGCATT
CGGGGGATCGTTACGACTAGCATCGATG
SEQ ID No. 60 >PB.97.126.I_48-C8
GGGAGAGGAGAGAACGTTCTCGTGCGACGGGCTTCTTGTGTCATTCGCAT
GGGATCGTTACGACTAGCATCGATG
SEQ ID No. 61 >PB.97.126.I_48-D8
GGGAGAGGAGAGAACGTTCTCGGCATTGCAGTTGATAGGTCGCGCAGTGC
TGGGATCGTTACGACTAGCATCGATG
SEQ ID No. 62 >PB.97.126.I_48-E8
GGGAGAGGAGAGAACGTTCTCGCGATATGCAGTCTGAGAAGTCGCGCATT
CGAGGGATCGTTACGACTAGCATCGATG
SEQ ID No. 63 >PB.97.126.I_48-F8
GGGAGAGGAGAGAACGTTCTCGTGTAGCAAGCATGTGGATCGCGACTGCA
CGGGATCGTTACGACTAGCATCGATG
SEQ ID No. 64 >PB.97.126.I_48-G8
GGGAGAGGAGAGAACGTTCTCGGATAAGCAGTTGAGATGTCGCGCTTTGA
CGGGATCGTTACGACTAGCATCGATG
SEQ ID No. 65 >PB.97.126.I_48-H8
GGGAGAGGAGAGAACGTTCTCGATGANCANTTTGAGAAGTCGCGCTTGTC
GGGATCGTTACGACTAGCATCGATG
SEQ ID No. 66 >PB.97.126.I_48-A9
GGGAGAGGAGAGAACGTTCTCGAGTAATGCAGTGGAAGTCGCGCATTACC
TGGGATCGTTACGACTAGCATCATG
SEQ ID No. 67 >PB.97.126.I_48-B9
GGGAGAGGAGAGAACGTTCTCGCGATATGCAGTTTGAGAAGTCGCGCATT
CGGGGGATCGTTACGACTAGCATCGATG
SEQ ID No. 68 >PB.97.126.I_48-C9
GGGAGAGGAGAGAACGTTCTCGTGATNCAGTTGANAAGTCNCGCATACAG
GATCGTTACGACTAGCATCGATG
SEQ ID No. 69 >PB.97.126.I_48-D9
GGGAGAGGAGAGAACGTTCTCGAGTAATGCTGTGGAAGTCGCGCATTTCC
TGGGATCGTTACGACTAGCATCGATG
SEQ ID No. 70 >PB.97.126.I_48-D8
GGGAGAGGAGAGAACGTTCTCGGCATTGCAGTTGATAGGTCGCGCAGTGC
TGGGATCGTTACGACTAGCATCGATG
SEQ ID No. 71 >PB.97.126.I_48-F9
GGGAGAGGAGAGAACGTTCTCGCGATATGCAGTTTGGGAAGTCGCGCATT
CGAGGGATCGTTACGACTAGCATCGATG
SEQ ID No. 72 >PB.97.126.I_48-G9
GGGAGAGGAGAGAACGTTCTCGCNATATGCTGTTTGANAANTCGCGCATT
CGGGGGATCGTTACGACTAGCATCGATG
SEQ ID No. 73 >PB.97.126.I_48-H9
GGGAGAGGAGAGAACGTTCTCGCGTAGATTGGGCTGAATGGGATATCTTT
AGCGGGATCGTTACGACTAGCATCGATG
SEQ ID No. 74 >PB.97.126.I_48-B10
GGGAGAGGAGAGAACGTTCTCGCGATATGCAGTTTGAGAAGTCGCGCTTT
CGAGGGATCGTTACGACTAGCATCGATG
SEQ ID No. 75 >PB.97.126.I_48-D10
GGGAGAGGAGAGAACGTTCTCGTCAATCTGATGTAGCCTCACGTGGGCGG
AGTCGGATCGTTACGACTAGCATCGATG

TABLE 5

Corresponding cDNAs of the VEGF Aptamer
Sequences - alternately “r/mGmH” and 2′-OMe
AUC, 2′-F G (toggle)

SEQ ID No. 76 >PB.97.126.J_48-F10
GGGAGAGGAGAGAACGTTCTCGGATCGTTACGACTAGCATCGATG
SEQ ID No. 77 >PB.97.126.J_48-G10
GGGAGAGGAGAGAACGTTCTCGGATCGTTACGACTAGCATCGATG
SEQ ID No. 78 >PB.97.126.J_48-H10
GGGAGAGGAGAGAACGTTCTCGGTGGTGTTGCTGAACTGTCGCGTTTCGC
CGGGATCGTTACGACTAGCATCGATG
SEQ ID No. 79 >PB.97.126.J_48-A11
GGGAGAGGAGAGAACGTTCTCGTCGCGATTGCATATTTTCCGCCTTGCTG
TGAGGATCGTTACGACTAGCATCGATG
SEQ ID No. 80 >PB.97.126.J_48-B11
GGGAGAGGAGAGAACGTTCTCGCGATTTGCAGTTTGAGATGTCGCGCATT
CGAGGGATCGTTACGACTAGCATCGATG
SEQ ID No. 81 >PB.97.126.J_48-C11
GGGAGAGGAGAGAACGTTCTCGCGATATGCAGTTTGAGAAGTCGCGCATT
CGGGGGATCGTTACGACTAGCATCGATG
SEQ ID No. 82 >PB.97.126.J_48-D11
GGGAGAGGAGAGAACGTTCTCGTTGGTGCAGTTTGAGATGTCGCGCACCT
TGGGATCGTTACGACTAGCATCGATG
SEQ ID No. 83 >PB.97.126.J_48-E11
GGGAGAGGAGAGAACGTTCTCGGTATTGGTTCCATTAAGCTGGACACTCT
GCTCCGGGATCGTTACGACTAGCATCGATG
SEQ ID No. 84 >PB.97.126.J_48-F11
GGGAGAGGAGAGAACGTTCTCGTTGGTGCAGTTTGAGATGTCGCGCGCCT
TGGGATCGTTACGACTAGCATCGATG
SEQ ID No. 85 >PB.97.126.J_48-G11
GGGAGAGGAGAGAACGTTCTCGCGATATGCAGTTTGAGAAGTCGCGCATT
CGAGGGATCGTTACNACTAGCATCGATG
SEQ ID No. 86 >PB.97.126.J_48-A12
GGGAGAGGAGAGAACGTTCTCGCGATATGCAGTTTGAGAAGTCGCGCATT
CGGGGGATCGTTACGACTAGCATCGATG
SEQ ID No. 87 >PB.97.126.J_48-B12
GGGAGAGGAGAGAACGCTCTCGGGGACNNAAANNCGAATTGNCGCGTGNG
TCCGGGGGAGCGCCCGACTAGTCATCGATG
SEQ ID No. 88 >PB.97.126.J_48-C12
GGGAGAGGAGAGAACGTTCTCGCGATATGNANTTTGAGAAGTCGCGCATT
CGGGGGATCGTTACGACTAGCATCGATG
SEQ ID No. 89 >PB.97.126.J_48-D12
GGGAGAGGAGAGAACGTTCTCGGTGTACAGCTTGAGATGTCGCGTACTCC
GGGATCGTTACGACTAGCATCGATG
SEQ ID No. 90 >PB.97.126.J_48-E12
GGGAGAGGAGAGAACGTTCTCGCGATATGCAGTTTGAGAAGTCGCGCATT
CGGGGGATCGTTACGACTAGCATCGATG
SEQ ID No. 91 >PB.97.126.J_48-F12
GGGAGAGGAGAGAACGTTCTCGAGTAAGAAAGCTGAATGGTCGCACTTCT
CGGGATCGTTACGACTAGCATCGATG
SEQ ID No. 92 >PB.97.126.J_48-G12
AGGGAGAGGAAGAACGTTCTCGCGATGTGCAGTTTGAGAAGTCGCGCATT
CGAGGGATCGTTACGACTAGCATCGATG
SEQ ID No. 93 >PB.97.126.J_48-H12
GGGAGAGGAGAGAACGTTCTCGAAAGAATCAGCATGCGGATCGCGGCTTT
CGGGATCGTTACGACTAGCATCGATG

TABLE 6
Corresponding cDNAs of the Thrombin Aptamer
Sequences - all 2′-OH (rN)

SEQ ID No. 94 >PB.97.126.A_44-A1
GGGAGAGGAGAGAACGTTCTCGANTCCANTNTNCNTGGAGGAGTAAGTAC
CTGAGGGATCGTTACGACTAGCATCGATG
SEQ ID No. 95 >PB.97.126.A_44-B1
GGGAGAGGAGAGAACGTTCTCGGGAAACAAGGAACTTAGAGTTANTTGAC
CGGGATCGTTACGACTAGCATCGATG
SEQ ID No. 96 >PB.97.126.A_44-C1
GGGAGAGGAGAGAACGTTCTCGTACCATGCAAGGAACATAATAGTTAGCG
TGGGATCGTTACGACTAGCATCGATG
SEQ ID No. 97 >PB.97.126.A_44-D1
GGGAGAGGAGAGAACGTTCTCGGGACACAAGGAACACAATAGTTAGTGTA
CGGGATCGTTACGACTAGCATCGATG
SEQ ID No. 98 >PB.97.126.A_44-E1
GGGAGAGGAGAGAACGTTCTCGTCTGCAAGGAACACAATAGTTAGCATTG
CGGGATCGTTACGACTAGCATCGATG
SEQ ID No. 99 >PB.97.126.A_44-F1
GGGAGAGGAGAGAACGTTCTCGCGCCAACAAAGCTGGAGTACTTAGAGCG
CGGGATCGTTACGACTAGCATCGATG
SEQ ID No. 100 PB.97.126.A_44-G1
GGGAGAGGAGAGAACGTTCTCGATTGCAAAATAGCTGTAGAACTAAGCAA
TCGGATCGTTACGACTAGCATCGATG
SEQ ID No. 101 >PB.97.126.A_44-H1
GGGAGAGGAGAGAACGTTCTCGTGAGATGACTATGTTAAGATGACGCTGT
TGGGATCGTTACGACTAGCATCGATG
SEQ ID No. 102 >PB.97.126.A_44-A2
GGGAGAGGAGAGAACGTTCTCGGGANACAAGGAACNCAATATTTAGTGAA
CGGGATCGTTACGACTAGCATCGATG
SEQ ID No. 103 >PB.97.126.A_44-B2
GGGAGAGGAGAGAACGTTCTCGCCAAGGAACACAATAGTTAGGTGAGAAT
CGGGATCGTTACGACTAGCATCGATG
SEQ ID No. 104 >PB.97.126.A_44-C2
GGGAGAGGAGAGACGTTCTCGGTACAAGGAACACAATAGTTAGTGCCGTG
GGATCGTTACGACTAGCATCGATG
SEQ ID No. 105 >PB.97.126.A_44-D2
GGGAGAGGAGAGAACGTTCTCGATTCAACGGTCCAAAAAAGCTGTAGTAC
TTAGGATCGTTACGACTAGCATCGATG
SEQ ID No. 106 >PB.97.126.A_44-E2
GGGAGAGGAGAGAACGTTCTCGCAATGCAAGGAACACAATAGTTAGCAGC
CGGGATCGTTACGACTAGCATCGATG
SEQ ID No. 107 >PB.97.126.A_44-F2
GGGAGAGGAGAGAACGTTCTCGAAAGGAGAAAGCTGAAGTACTTACTATG
CGGGATCGTTACGACTAGCATCGATG
SEQ ID No. 108 >PB.97.126.A_44-G2
GGGAGAGGAGAGAACGTTCTCGCACAAGGAACACAATAGTTAGTGCAAGA
CGGGATCGTTACGACTAGCATCGATG
SEQ ID No. 109 >PB.97.126.A_44-A3
GGGAGAGGAGAGAACGTTCTCGCACAAGGAACTACGAGTTAGTGTGGGAG
TGGGATCGTTACGACTAGCATCGATG
SEQ ID No. 110 >PB.97.126.A_44-B3
GGGAGAGGAGAGAACGTTCTCGCACAAGGAACACAATAGTTAGTGCAAGA
CGGGATCGTTACGACTAGCATCGATA
SEQ ID No. 111 >PB.97.126.A_44-C3
GGGAGAGGAGAGAACGTTCTCGGCGGGAAAATAGCTGTAGTACTAACCCA
CGGATCGTTACGACTAGCATCGATG

TABLE 7

Corresponding cDNAs of the Thrombin Aptamer
Sequences - 2′-OH AG, 2′-OMe CU (rRmY)

SEQ ID No. 112 >PB.97.126.B_44-E3
GGGAGAGGAGAGAACGTTCTCGGCCTCAAGGAAAAGAAAATTTAGAGGCC
CGGGATCGTTACGACTAGCATCGATG
SEQ ID No. 113 >PB.97.126.B_44-F3
GGGAGAGGAGAGAACGTTCTCGGAACAAGATAGCTGAAGGACTAAGTTTA
CGGGATCGTTACGACTAGCATCGATG
SEQ ID No. 114 >PB.97.126.B_44-G3
GGGAGAGGAGAGAACGTTCTCGGAACAAGATAGCTGAAGGACTAAGTTTA
CGGGATCGTTACGACTAGCATCGATG
SEQ ID No. 115 >PB.97.126.B_44-H3
GGGAGAGGAGAGAACGTTCTCGGAGCCAAGGAAACGAAGATTTAGGCTCA
TTGGGATCGTTACGACTAGCATCGATG
SEQ ID No. 116 >PB.97.126.B_44-A4
GGGAGAGGAGAGAACGTTCTCGATCACAAGAAATGTGGGANGGTAGTGAT
NCNNNTCGTTNCGACTAGCATCGATG
SEQ ID No. 117 >PB.97.126.B_44-B4
GGGAGAGGAGAGAACGTTCTCGTCGAAAGGGAGCTTTGTCTCGGGACAGA
ACGGATCGTTACGACTAGCATCGATG
SEQ ID No. 118 >PB.97.126.B_44-C4
GGGAGAGGAGAGAACGNTCTCGTGCAAAGATAGCTGGAGGACTAATGCGG
CGGGATCGTTACGACTAGCATCGATG
SEQ ID No. 119 >PB.97.126.B_44-D4
GGGAGAGGAGAGAACGTTCTCGTCGAAAGGGAGCTTTGTCTCGGGACAGA
ACGGATCGTTACGACTAGCATCGATG
SEQ ID No. 120 >PB.97.126.B_44-E4
GGGAGAGGAGAGAACGTTCTCGNCNAAGGNGAGCTTTGTCCCNGGACANA
ANGNATCGTTACAACTAGCATCGATG
SEQ ID No. 121 >PB.97.126.B_44-F4
GGGAGAGGAGAGAACGTTCTCGGAACAAGATAGCTGAAGGACTAAGTTTA
CGGGATCGTTACGACTAGCATCGATG
SEQ ID No. 122 >PB.97.126.B_44-G4
GGGAGAGGAGAGAACGTTCTCGGAACAAGATAGCTGAAGGACTAAGTTTA
CGGGATCGTTACGACTAGCATCGATG
SEQ ID No. 123 >PB.97.126.B_44-H4
GGGAGAGGAGAGAACGTTCTCGGCGCAAAAAAAGCTGGAGTACTTAGTGT
CGAGGGATCGTTACGACTAGCATCGATG
SEQ ID No. 124 >PB.97.126.B_44-A5
GGGAGAGGAGAGAACGTTCTCGTCGAAAGGGAGCTTTGTCTCGGGACAGA
ACGGATCGTTACGACTAGCATCGATG
SEQ ID No. 125 >PB.97.126.B_44-B5
GGGAGAGGAGAGAACGTTCTCGACACAAGAAAGCTGCAGAACTTAGGGTC
GTGGATCGTTACGACTAGCATCGATG
SEQ ID No. 126 >PB.97.126.B_44-C5
GGGAGAGGAGAGAACGTTCTCGGAACNGGATTGTTGAAGGACTAANTTTA
CGGGATCGTTACGACTAGCATCGATG
SEQ ID No. 127 >PB.97.126.B_44-D5
GGGAGAGGAGAGAACGTTCTCGGCCTCAAGGGAAAGAAAATTTAGAGGCC
CGGGATCGTTACGACTAGCATCGATG
SEQ ID No. 128 >PB.97.126.B_44-E5
GGGAGAGGAGAGAACGTTCTCGGAAACAAGCTTAGAAATTCGCACCCTTG
CCGGGATCGTTACGACTAGCATCGATG
SEQ ID No. 129 >PB.97.126.B_44-F5
GGGAGAGGAGAGAACGTTCTCGAAAGAAAAAAGCTGGAGAACTTACTTCC
GGGATCGTTACGACTAGCATCGATG
SEQ ID No. 130 >PB.97.126.B_44-G5
GGGAGAGGAGAGAACGTTCTCGGTGATTGTACTCACATAGAAATGGCAAC
ACTGGGATCGTTACGACTAGCATCGATG

TABLE 8

Corresponding cDNAs of the Thrombin Aptamer
Sequences - 2′-OH G, 2′-OMe CUA (rGmH)

SEQ ID No. 132 >PB.97.126.C_44-H5
GGGAGAGGAGAGAACGTTCTCGGGTTCAAGGAACATGATAGTTAGAACCC
GCGGATCGTTACGACTAGCATCGATG
SEQ ID No. 132 >PB.97.126.C_44-A6
GGGAGAGGAGAGAACGTTCTCGTTCCGAAAGGAACACAATAGTTATCGGA
TTGGGATCGTTACGACTAGCATCGATG
SEQ ID No. 133 >PB.97.126.C_44-B6
GGGAGAGGAGAGACGTTCTCGTCTGCAAGGAACACAATAGTTAGCATTGC
GGGATCGTTACGACTAGCATCGATG
SEQ ID No. 134 >PB.97.126.C_44-C6
GGGAGAGGAGAGAACGTTCTCGGTACAAGGAACACAATAGTTAGTGCCGG
GGATCGTTACGACTAGCATCGATG
SEQ ID No. 135 >PB.97.126.C_44-D6
GGGAGAGGAGAGAACGTTCTCGGAACTCAGAGATCCTATGTGGACCAGAG
AGGATCGTTACGACTAGCATCGATG
SEQ ID No. 136 >PB.97.126.C_44-E6
GGGAGAGGAGAGAACGTTCTCGCTGAGCAAGGAACGTAATAGTTAGCCTG
CGGGATCGTTACGACTAGCATCGATG
SEQ ID No. 137 >PB.97.126.C_44-F6
GGGAGAGGAGAGAACGTTCTCGNANNNATAAATGATGGATCNCTTATTG
TNNAGGATCGTTACGACTAGCATCGATG
SEQ ID No. 138 >PB.97.126.C_44-G6
GGGAGAGGAGAGAACGTTCTCGGCTTGGAAAAATAGCTTTTGGGCATCC
GGGATCGTTACGACTAGCATCGATG
SEQ ID No. 139 >PB.97.126.C_44-H6
GGGAGAGGAGAGAACGTTCTCGGGTTCAAGGAACATGATAGCTAGAACC
CGCGGATCGTTACGACTAGCATCGATG
SEQ ID No. 140 >PB.97.126.C_44-A7
GGGAGAGGAGAGAACGTTCTCGGGTTCAAGGAACATGATAGTTAGAACC
CGCGGATCGTTACGACTAGCATCGATG
SEQ ID No. 141 >PB.97.126.C_44-B7
GGGAGAGGAGAGAACGTTCTCGTGGGCAGGGAACACAATAGTTAGCCTA
CGCGGATCGTTACGACTAGCATCGATG
SEQ ID No. 142 >PB.97.126.C_44-C7
GGGAGAGGAGAGAACGTTCTCGCGTGAAAGGAACACAATAGTTATCGTG
CGGGATCGTTACGACTAGCATCGATG
SEQ ID No. 143 >PB.97.126.C_44-D7
GGGAGAGGAGAGAACGTTCTCGCGAGGTTTATCCTAGACGACTAACCGC
CTGGGGATCGTTACGACTAGCATCGATG
SEQ ID No. 144 >PB.97.126.C_44-F7
GGGAGAGGAGAGAACGTTCTCGTCTGCTAGGAACACAATAGTTAGCATT
GCGGGATCGTTACGACTAGCATCGATG
SEQ ID No. 145 >PB.97.126.C_44-G7
GGGAGAGGAGAGAACGTTCTCGCACAAGGAACTACGAGTTAGTGTGGGA
GTGGGGATCGTTACGACTAGCATCGATG
SEQ ID No. 146 >PB.97.126.C_44-H7
GGGAGAGGAGAGAACGTTCTCGTGACACGAGGAACTTAGAGTTAGTAGC
ACGAGGATCGTTACGACTAGCATCGATG
SEQ ID No. 147 >PB.97.126.C_44-A8
GGGAGAGGAGAGAACGTTCTCGGCGGCGAAGGAACACAATAGTTACGTC
CCGGGATCGTTACGACTAGCATCGATG
SEQ ID No. 148 >PB.97.126.C_44-B8
GGGAGAGGAGAGAACGTTCTCGAGCCCAAAAAAGCTGAAGTACTTTGGG
CAGGGATCGTTACGACTAGCATCGATG

TABLE 9

Corresponding cDNAs of the Thrombin Aptamer
Sequences - 2′-OMe AUGC (r/mGmH, each G has a 90%
probability of having a 2′-OMe group
incorporated therein)

SEQ ID No. 149 >PB.97.126.D_44-D8
GGGAGAGGAGAGAACGTTCTCGGTACAAGGAACACAATAGTTAGTGCCG
TGGGATCGTTACGACTAGCATCGATG
SEQ ID No. 150 >PB.97.126.D_44-E8
GGGAGAGGAGAGAACGTTCTCGGATCGTTACGACTAGCATCGATG
SEQ ID No. 151 >PB.97.126.D_44-G8
GGGAGAGGAGAGAACGTTCTCGTGCGCAAGGAACACAATAGTTAGGGCG
CGAGGATCGTTACGACTAGCATTGATG
SEQ ID No. 152 >PB.97.126.D_44-H8
GGGAGAGGAGAGAACGTTCTCGGAATGGAAGGAACACAATAGTTACCAG
ACGGGATCGTTACGACTAGCATCGATG
SEQ ID No. 153 >PB.97.126.D_44-A9
GGGAGAGGAGAGAACGTTCTCGTCTGCAAGGAACACAATAGTTAGCATT
GCGGGATCGTTACGACTAGCATCGATG
SEQ ID No. 154 >PB.97.126.D_44-B9
GGGAGAGGAGAGAACGTTCTCGAGACAAGACAGCTGGAGGACTAAGTCA
CGAGGATCGTTACGACTAGCATCGATG
SEQ ID No. 155 >PB.97.126.D_44-C9
GGGAGAGGAGAGAACGTTCTCGATGCCCGCAAAGGAACACGATAGTTAT
GCGGGATCGTTACGACTAGCATCGATG
SEQ ID No. 156 >PB.97.126.D_44-D9
GGGAGAGGAGAGAACGTTCTCGTCTGNNAGGAACACAATATTTAGCATT
GCGGGATCGTTACGACTAGCATCGATG
SEQ ID No. 157 >PB.97.126.D_44-E9
GGGAGAGGAGAGAACGTTCTCGAATGTGCGGAGCAGTATTGGTACACTT
TCGGGATCGTTACGACTAGCATCGATG
SEQ ID No. 158 >PB.97.126.D_44-F9
GGGAGAGGAGAGAACGTTCTCGCCAAGGAACACAATAGTTAGGTGAGAA
TCGGGATCGTTACGACTAGCATCGATG
SEQ ID No. 159 >PB.97.126.D_44-G9
GGGAGAGGAGAGAACGTTCTCGCCAAGGAACACAATAGTTAGGTGAGAA
TCGGGATCGTTACGACTAGCATCGATG
SEQ ID No. 160 >PB.97.126.D_44-H9
GGGAGAGGAGAGAACGTTCTCGGGAAGCAAGGAACTTAGAGTTAGTTGA
CCGGGATCGTTACGACTAGCATCGATG
SEQ ID No. 161 >PB.97.126.D_44-A10
GGGAGAGGAGAGAACGTTCTCGTGGGCAAGGAACACAATAGTTAGCCTA
CGCGGATCGTTACGACTAGCATCGATG
SEQ ID No. 162 >PB.97.126.D_44-B10
GGGAGAGGAGAGAACGTTCTCGTCGGGCATGGAACACAATAGTTAGACC
GCGGGATCGTTACGACTAGCATCGATG
SEQ ID No. 163 >PB.97.126.D_44-C10
GGGAGAGGAGAGAACGTTCTCGGTCGCAAGGAACATAATAGTTAGCGGA
GGGGATCGTTACGACTAGCATCGATG
SEQ ID No. 164 >PB.97.126.D_44-D10
GGGAGAGGAGAGAACGTTCTCGTCTGCAAGGAACACAATAGTTAGCATT
GCGGGATCGTTACGACTAGCATCGATG
SEQ ID No. 165 >PB.97.126.D_44-E10
GGGAGAGGAGAGAACGTTCTCGCCGACAATCAGCTCGGATCGTGTGCTA
CGCTGGATCGTTACGACTAGCATCGATG

TABLE 10

Corresponding cDNAs of the Thrombin Aptamer
Sequences - alternately “r/mGmH” and 2′-OMe AUC,
2′-F G (toggle).

SEQ ID No. 166 >PB.97.126.E_44-F10
GGGAGAGGAGAGAACGTTCTCGAGACAAGATAGCTGAAGGACTAAGTCA
CGAGGGATCGTTACGACTAGCATCGATG
SEQ ID No. 167 >PB.97.126.E_44-G10
GGGAGAGGAGAGAACGTTCTCGGAACAAGATAGCTGAAGGACTAAGTTT
GCGGGATCGTTACGACTAGCATCGATG
SEQ ID No. 168 >PB.97.126.E_44-H10
GGGAGAGGAGAGAACGTTCTCGGAGNCAAGGAAACNAATATTTAGGCTC
ANTGGNNNCNTTNCANCTAGCNNCNNTA
SEQ ID No. 169 >PB.97.126.E_44-A11
GGGAGAGGAGAGAACGTTCTCGTCTGCAAGGAACACAATAGTTAGCATT
GCGGGATCGTTACGACTAGCATCGATG
SEQ ID No. 170 >PB.97.126.E_44-B11
GGGAGAGGAGAGAACGTTCTCGGAACAAGATAGCTGAAGGACTAAGTTT
ACGGGATCGTTACGACTAGCATCGATG
SEQ ID No. 171 >PB.97.126.E_44-C11
GGGAGAGGAGAGAACGTTCTCGGATCGTTACGACTAGCATCGATG
SEQ ID No. 172 >PB.97.126.E_44-D11
GGGAGAGGAGAGAACGTTCTCGGTGATAGTACTCACATAGAAATGGCTA
CACTGGGATCGTTACGACTAGCATCGATG
SEQ ID No. 173 >PB.97.126.E_44-E11
GGGAGAGGAGAGAACGTTCTCGCCTGGGCAAGGAACAGAAAAGTTAGCG
CCAGGATCGTTACGACTAGCATCGATG
SEQ ID No. 174 >PB.97.126.E_44-F11
GGGAGAGGAGAGAACGTTCTCGTAACGGACAAAAGGAACCGGGAAGTTA
TCTGGATCGTTACGACTAGCATCGATG
SEQ ID No. 175 >PB.97.126.E_44-G11
GGGAGAGGAGAGAACGTTCTCGCGCACAAGATAGAGAAGACTAAGTCCG
CGGGGATCGTTACGACTAGCATCGATG
SEQ ID No. 176 >PB.97.126.E_44-H11
GGGAGAGGAGAGAACGTTCTCGCGCACAAGATAGAGAAGACTAAGTTCG
CGGGGATCGTTACGACTAGCATCGATG
SEQ ID No. 177 >PB.97.126.E_44-A12
GGGAGAGGAGAGAACGTTCTCGCGCCAATAAAGCTGGAGTACTTAGAGC
GCGGGATCGTTACGACTAGCATCGATG
SEQ ID No. 178 >PB.97.126.E_44-B12
GGGAGAGGAGAGAACGTTCTCGGGAAACAAGGAACTTAGAGTTAGTTGA
CCGGGATCGTTACGACTAGCATCGATG
SEQ ID No. 179 >PB.97.126.E_44-C12
GGGAGAGGAGAGAACGTTCTCGCTAGCAAGATAGGTGGGACTAAGCTAG
TGAGGATCGTTACGACTAGCATCGATG
SEQ ID No. 180 >PB.97.126.E_44-D12
GGGAGAGGAGAGAACGTTCTCGTCGAAGGGGAGCTTTGTCTCGGGACAG
AACGGATCGTTACGACTAGCATCGATG
SEQ ID No. 181 >PB.97.126.E_44-E12
GGGAGAGGAGAGAACGTTCTCGGAACAAGATAGCTGAAGGACTAAGTTT
ACGGGATCGTTACGACTAGCATCGATG
SEQ ID No. 182 >PB.97.126.E_44-G12
GGGAGAGGAGAGAACGTTCTCGGAACAAGATAGCTGAAGGACTAAGTTT
GCGGGATCGTTACGACTAGCATCGATG
SEQ ID No. 183 >PB.97.126.E_44-H12
GGGAGAGGAGANNTCCCCNCNCGGAAAAANAAAAAAGAAGAANTANGTT
NGGGGGATCGTTACGACTAGCATCGATG

TABLE 11

Stabilized Aptamer Sequences (each G residue has
90% probability of being substituted with a 2′-OMe
group, “3T” refers to an inverted thymidine
nucleotide attached to the phosphodiester backbone
at the 5′ position, the resulting oligo having two
5′-OH ends and is thus resistant to 3′ nucleases).

SEQ ID No. 184 ARC224 - Stabilized VEGF Aptamer
5′mCmGmAmUmAmUmGmCmAmGmUmUmUmGmAmGmAmAmGmUmCmGmCm
GmCmAmUmUmCmG-3T
SEQ ID No. 185 ARC225 - Stabilized VEGF Aptamer
5′mCmGmAmUmAmUGmCmAGmUmUmUGmAGmAmAGmUmCGmCGmCmAmUm
UmCmG-3T
SEQ ID No. 186 ARC226 Single-hydroxy VEGF aptamer
5′mGmAmUmCmAmUmGmCmAmUGmUmGmGmAmUmCmGmCmGmGmAmUmC-
3T
SEQ ID No. 187 ARC245 VEGF Aptamer
5′mAmUmGmCmAmGmUmUmUmGmAmGmAmAmGmUmCmGmCmGmCmAmU-
3T
SEQ ID No. 188 ARC259 hVEGF Aptamer - C-G base
pair swap of ARC245 (2nd base pair in) which has
improved binding over ARC245.
5′mAmCmGmCmAmGmUmUmUmGmAmGmAmAmGmUmCmGmCmGmCmGmU-
3′

### Example 2

### 2′-OMe SELEX™

Libraries of transcription templates were used to generate pools of RNA oligonucleotides incorporating 2′-O-methyl NTPs under various transcription conditions. The transcription template (ARC256) and the transcription conditions are described below as rRmY (SEQ ID NO:456), rGmH (SEQ ID NO:462), r/mGmH (SEQ ID NO:463), and dRmY (SEQ ID NO:464). The unmodified RNA transcript is represented by SEQ ID NO:468.

| | |
|---|---|
| ARC256: DNA transcription template | |
| 5′-CATCGATCGATCGATCGACAGCGNNNNNNNN | (SEQ ID NO:453) |
| NNNNNNNNNNNNNNNNNNNNNNGTAGAACGTTCT | |
| CTCCTCTCCCTATAGTGAGTCGTATTA-3′ | |

The ARC256 RNA transcription product is:

| | |
|---|---|
| 5′-GGGAGAGGAGAGAACGUUCUACNNNNNNNNN | (SEQ ID NO:468) |
| NNNNNNNNNNNNNNNNNNNNNCGCUGUCGAUCGA | |
| UCGAUCGAUG-3′ | |

The transcription conditions were varied as follows where 1×Tc buffer is 200 mM HEPES, 40 mM DTT, 2 mM Spermidine, 0.01% Triton X-100, pH 7.5.

When 2′-OMe C and U and 2′-OH A and G (rRmY) conditions were used, the transcription reaction conditions were 1×Tc buffer, 50-200 nM double stranded template (200 nm template was used for round 1, and for subsequent rounds approximately 50 nM, a 1/10 dilution of an optimized PCR reaction, using conditions described herein, was used), 9.6 mM MgCl₂, 2.9 mM MnCl₂, 2 mM each base, 10% PEG-8000, 0.25 units inorganic pyrophosphatase, and 1.5 units Y639F/H784A T7 RNA polymerase. One unit of the Y639F/H784A mutant T7 RNA polymerase is defined as the amount of enzyme required to incorporate 1 nmole of 2′-OMe NTPs into transcripts under the r/mGmH conditions. One unit of inorganic pyrophosphatase is defined as the amount of enzyme that will liberate 1.0 mole of inorganic orthophosphate per minute at pH 7.2 and 25° C.

When 2′-OMe A, C, and U and 2′-OH G (rGmH) conditions were used, the transcription reaction conditions were 1×Tc buffer, 50-200 nM double stranded DNA template (200 nm template was used for round 1, and for subsequent rounds approximately 50 nM, a 1/10 dilution of an optimized PCR reaction, using conditions described herein was used), 9.6 mM MgCl₂, 2.9 mM MnCl₂, 2 mM each base, 10% PEG-8000, 0.25 units inorganic pyrophosphatase, and 1.5 units Y639F single mutant T7 RNA polymerase. One unit of the Y639F mutant T7 RNA polymerase is defined as the amount of enzyme required to incorporate 1 nmole of 2′-OMe NTPs into transcripts under the r/mGmH conditions.

When all 2′-OMe nucleotides (r/mGmH) conditions were used, the reaction conditions were 1×Tc buffer, 50-200 nM double stranded template (200 nm template was used for round 1, and for subsequent rounds approximately 50 nM, a 1/10 dilution of an optimized PCR reaction, using conditions described herein was used), 6.5 mM MgCl₂, 2 mM MnCl₂, 1 mM each base, 30 μM GTP, 1 mM GMP, 10% PEG-8000, 0.25 units inorganic pyrophosphatase, and 1.5 units Y639F/H784A T7 RNA polymerase.

When deoxy purines, A and G, and 2′-OMe pyrimidines (dRmY) conditions were used, the reaction conditions were 1×Tc buffer, 50-300 nM double stranded template (300 nm template was used for round 1, and for subsequent rounds approximately 50 nM, a 1/10 dilution of an optimized PCR reaction, using conditions described herein was used), 9.6 mM MgCl₂, 2.9 mM MnCl₂, 2 mM each base, 30 μM GTP, 2 mM Spermine, 10% PEG-8000, 0.25 units inorganic pyrophosphatase, and 1.5 units Y639F single mutant RNA polymerase.

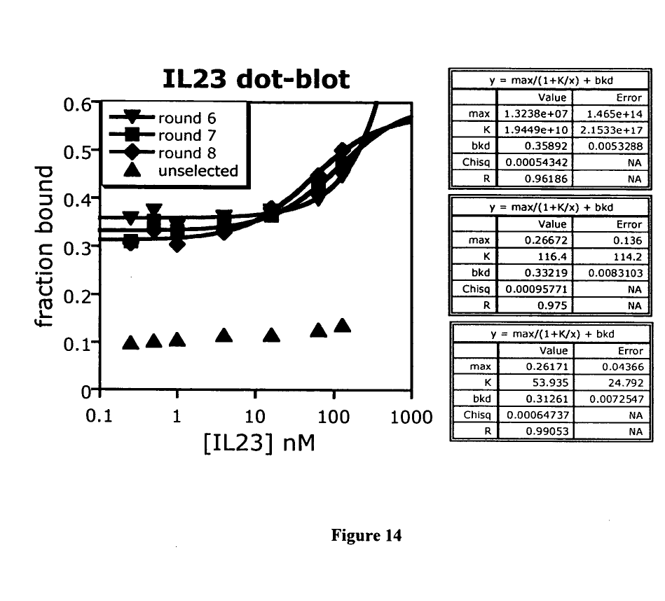

These pools were then used in SELEX™ to select for aptamers against the following targets: IgE, IL-23, PDGF-BB, thrombin and VEGF. A plot of dRmY Round 6, 7, 8, and unselected sequences binding to target IL-23 is shown in FIG. 14, and a plot of dRmY Round 6, 7, and unselected sequences binding to target PDGF-BB is shown in FIG. 14.

### Example 3

### dRmY SELEX™ of Aptamers Against IgE

While fully 2′-OMe substituted oligonucleotides are the most stable modified aptamers, substituting the purines with deoxy purine nucleotides also results in stable transcripts. When dRmY (deoxy purines, A and G, and 2′-OMe pyrimidines) transcription conditions are used, the products are very DNase-resistant and useful as stable therapeutics. This result is surprising since the composition of the dRmY transcripts is approximately 50% DNA, which is notoriously easily degraded by nucleases. Also, when dRmY transcription conditions are used, there is no requirement for a 2′-OH GTP spike. Studies have shown that approximately the same amount of dRmY transcripts having modified nucleotides are produced with 2′-OH GTP doping as without 2′-OH GTP doping. Accordingly, under dRmY transcription conditions, 2′-OH GTP doping is optional. Libraries of transcription templates were used to generate pools of oligonucleotides incorporating 2′-O-methylpyrimidine NTPs (U and C) and deoxy purines (A and G) NTPs under various transcription conditions. The transcription template (ARC256) and the transcription conditions are described below as dRmY.

| | |
|---|---|
| ARC256: DNA transcription template | |
| 5′-CATCGATCGATCGATCGACAGCGNNNNNNNN | (SEQ ID NO:453) |
| NNNNNNNNNNNNNNNNNNNNNNGTAGAACGTTCT | |
| CTCCTCTCCCTATAGTGAGTCGTATTA-3′ | |

The ARC256 dRmY RNA transcription product is:

| | |
|---|---|
| 5′-GGGAGAGGAGAGAACGUUCUACNNNNNNNNN | (SEQ ID NO:464) |
| NNNNNNNNNNNNNNNNNNNNNCGCUGUCGAUCGA | |
| UCGAUCGAUG-3′ | |

When deoxy purines, A and G, and 2′-OMe pyrimidines (dRmY) conditions were used, the reaction conditions were 1×Tc buffer, 50-300 nM double stranded template (300 nm template was used for round 1, and for subsequent rounds approximately 50 nM, a 1/10 dilution of an optimized PCR reaction, using conditions described herein, was used), 9.6 mM MgCl₂, 2.9 mM MnCl₂, 2 mM each base, 30 μM GTP, 2 mM Spermine, 10% PEG-8000, 0.25 units inorganic pyrophosphatase, and 1.5 units Y639F single mutant RNA polymerase.

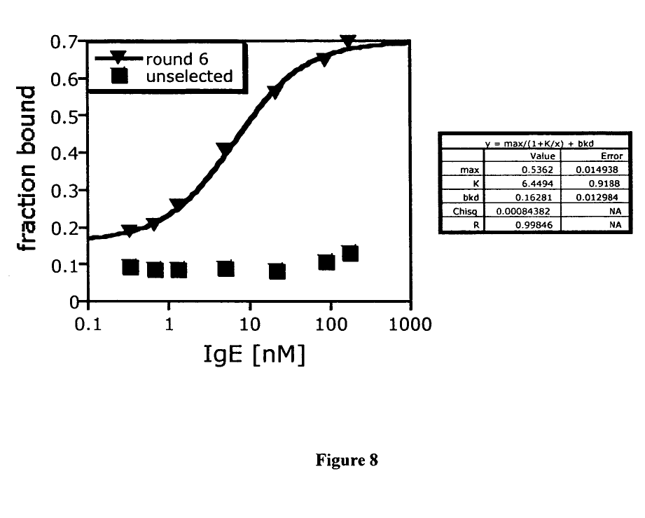

These pools were then used in SELEX™ to select for aptamers against IgE as a target. The sequences obtained after round 6 of SELEX™ as described above are listed in Table 12 below. A plot of Round 6 sequences bound with increasing target IgE concentration is shown in FIG. 8.

TABLE 12

Corresponding cDNAs of the Round 6 sequences of
dRmY SELEX ™ against IgE.

SEQ ID No.190 IgE A5
GGGAGAGGAGAGAACGTTCTACAGGCAGTTCTGGGGACCCATGGGGGAAG
TGCGCTGTCGATCGATCGATCGATG
SEQ ID No.191 IgE A6
GGGAGAGGAGAGAACGTTCTACGATTAGCAGGGAGGGAGAGTGCGAAGAG
GACGCTGTCGATCGATCGATCGATG
SEQ ID No.192 IgE A7
GGGAGAGGAGAGAACGTTCTACACTCTGGGGACCCGTGGGGGAGTGCAG
CAACGCTGTCGATCGATCGATCGATG
SEQ ID No.193 IgE A8
GGGAGAGGAGAGAACGTTCTACAAGCAGTTCTGGGGACCCATGGGGGAA
GTGCGCTGTCGATCGATCGATCGATG
SEQ ID No.194 IgE B5
GGGAGAGGAGAGAACGTTCTACGAGGTGAGGGTCTACAATGGAGGGATG
GTCGCTGTCGATCGATCGATCGATG
SEQ ID No.195 IgE B6
GGGAGAGGAGAGAACGTTCTACCCGCAGCATAGCCTGNGGACCCATGNG
GGGCGCTGTCGATCGATCGATCGATG
SEQ ID No.196 IgE B7
GGGAGAGGAGAGAACGTTCTACTGGGGGGCGTGTTCATTAGCAGCGTCG
TGTCGCTGTCGATCGATCGATCGATG
SEQ ID No.197 IgE B8
GGGAGAGGAGAGAACGTTCTACAGGCAGTTCTGGGGACCCATGGGGGAA
GTGCGCTGTCGATCGATCGATCGATG
SEQ ID No.198 IgE C5
GGGAGAGGAGAGAACGTTCTACGCAGCGCATCTGGGGACCCAAGAGGGG
ATTCGCTGTCGATCGATCGATCGATG
SEQ ID No.199 IgE C6
GGGAGAGGAGAGAACGTTCTACAGGCAGTTCTGGGGACCCATGGGGGAA
GTGCGCTGTCGATCGATCGATCGATG
SEQ ID No.200 IgE C7
GGGAGAGGAGAGAACGTTCTACGGGATGGGTAGTTGGATGGAAATGGGA
ACGCTGTCGATCGATCGATCGATG
SEQ ID No.201 IgE C8
GGGAGAGGAGAGAACGTTCTACGAGGTGTAGGGATAGAGGGGTGTAGGT
AACGCTGTCGATCGATCGATCGATG
SEQ ID No.202 IgE D5
GGGAGAGGAGAGAACGTTCTACAGGAGTGGAGCTACAGAGAGGGTTAGG
GGTCGCTGTCGATCGATCGATCGATG
SEQ ID No.203 IgE D6
GGGAGAGGAGAGAACGTTCTACGGATGTTGGGAGTGATAGAAGGAAGGG
GAGCGCTGTCGATCGATCGATCGATG
SEQ ID No.204 IgE D7
GGGAGAGGAGAGAACGTTCTACAGGCAGTTCTGGGGACCCATGGGGGAA
GTGCGCTGTCGATCGATCGATCGATG
SEQ ID No.205 IgE D8
GGGAGAGGAGAGAACGTTCTACAGGCAGTTCTGGGGACCCATGGGGGAA
GTGCGCTGTCGATCGATCGATCGATG
SEQ ID No.206 IgE E5
GGGAGAGGAGAGAACGTTCTACAGGCAGTTCTGGGGACCCATGGGGGAA
GTGCGCTGTCGATCGATCGATCGATG
SEQ ID No.207 IgE E6
GGGAGAGGAGAGAACGTTCTACTTGGGGTGGAAGGAGTAAGGGAGGTGC
TGATCGCTGTCGATCGATCGATCGATG
SEQ ID No.208 IgE E7
GGGAGAGGAGAGAACGTTCTACGTATTAGGGGGGAAGGGGAGGAATAGA
TCACGCTGTCGATCGATCGATCGATG
SEQ ID No.209 IgE E8
GGGAGAGGAGAGAACGTTCTACAGGGAGAGAGTGTTGAGTGAAGAGGAG
GAGTCGCTGTCGATCGATCGATCGATG
SEQ ID No.210 IgE F5
GGGAGAGGAGAGAACGTTCTACATTGTGCTCCTGGGGCCCAGTGGGGAG
CCACGCTGTCGATCGATCGATCGATG
SEQ ID No.211 IgE F6
GGGAGAGGAGAGAACGTTCTACGAGCAGCCCTGGGGCCCGGAGGGGGAT
GGTCGCTGTCGATCGATCGATCGATG
SEQ ID No.212 IgE F7
GGGAGAGGAGAGAACGTTCTACAGGCAGTTCTGGGGACCCATGGGGGAA
GTGCGCTGTCGATCGATCGATCGATG
SEQ ID No.213 IgE F8
GGGAGAGGAGAGAACGTTCTACCAACGGCATCCTGGGCCCCACAGGGGA
TGTCGCTGTCGATCGATCGATCGATG
SEQ ID No.214 IgE G5
GGGAGAGGAGAGAACGTTCTACGAGTGGATAGGGAAGAAGGGGAGTAGT
CACGCTGTCGATCGATCGATCGATG
SEQ ID No.215 IgE G6
GGGAGAGGAGAGAACGTTCTACCCGCAGCATAGCCTGGGGACCCATGGG
GGGCGCTGTCGATCGATCGATCGATG
SEQ ID No.216 IgE G7
GGGAGAGGAGAGAACGTTCTACGGTCGCGTGTGGGGGACGGATGGGTAT
TGGTCGCTGTCNATCGATCGATCNATG
SEQ ID No.217 IgE G8
GGGAGAGGAGAGAACGTTCTACCCGCAGCATAGCCTGGGGACCCATGGG
GGGCGCTGTCGATCGATCGATCGATG
SEQ ID No.218 IgE H5
GGGAGAGGAGAGAACGTTCTACCCGCAGCATAGCCTGGGGACCCATGGG
GGGCGCTGTCGATCGATCGATCGATG
SEQ ID No.219 IgE H6
GGGAGAGGAGAGAACGTTCTACGGGGTTACGTCGCACGATACATGCATT
CATCGCTGTCGATCGATCGATCGATG
SEQ ID No.220 IgE H7
GGGAGAGGAGAGAACGTTCTACTAGCGAGGAGGGGTTTTCTATTTTTGC
GATCGCTGTCGATCGATCGATCGATG

### Example 4

### dRmY SELEX™ of Aptamers Against Thrombin

While fully 2′-OMe substituted oligonucleotides are the most stable modified aptamers, substituting the purines with deoxy purine nucleotides also results in stable transcripts. When dRmY (deoxy purines, A and G, and 2′-OMe pyrimidines) transcription conditions are used, the products are very DNase-resistant and useful as stable therapeutics. This result is surprising since the composition of the dRmY transcripts is approximately 50% DNA, which is notoriously easily degraded by nucleases. Also, when dRmY transcription conditions are used, there is no requirement for a 2′-OH GTP spike. Libraries of transcription templates were used to generate pools of oligonucleotides incorporating 2′-O-methylpyrimidine NTPs (U and C) and deoxy purines (A and G) NTPs under various transcription conditions. The transcription template (ARC256) and the transcription conditions are described below as dRmY.

| | |
|---|---|
| ARC256: DNA transcription template | |
| 5′-dCATCGATCGATCGATCGACAGCGNNNNNNN | (SEQ ID NO:453) |
| NNNNNNNNNNNNNNNNNNNNNNNGTAGAACGTTC | |
| TCTCCTCTCCCTATAGTGAGTCGTATTA-3′ | |

The ARC256 dRmY RNA transcription product is:

| | |
|---|---|
| 5′-GGGAGAGGAGAGAACGUUCUACNNNNNNNNN | (SEQ ID NO:464) |
| NNNNNNNNNNNNNNNNNNNNNCGCUGUCGAUCGA | |
| UCGAUCGAUG-3′ | |

When deoxy purines, A and G, and 2′-OMe pyrimidines (dRmY) conditions were used, the reaction conditions were 1×Tc buffer, 50-300 nM double stranded template (300 nm template was used for round 1, and for subsequent rounds a 1/10 dilution of an optimized PCR reaction, using conditions described herein, was used), 9.6 mM MgCl₂, 2.9 mM MnCl₂, 2 mM each base, 30 μM GTP, 2 mM Spermine, 10% PEG-8000, 0.25 units inorganic pyrophosphatase, and 1.5 units Y639F single mutant RNA polymerase.

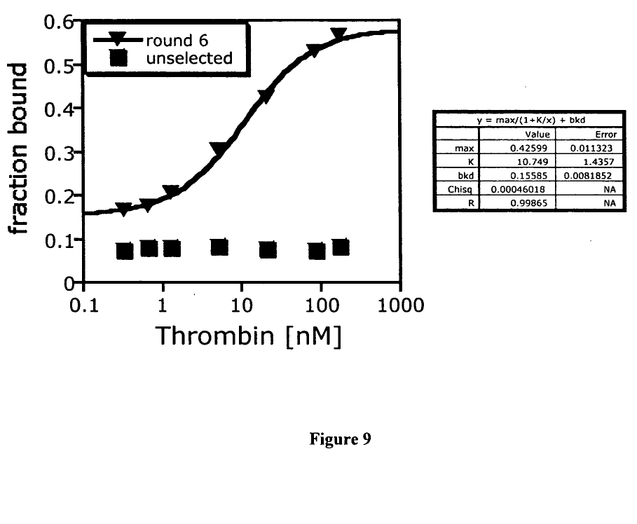

These pools were then used in SELEX™ to select for aptamers against thrombin as a target. The sequences obtained after round 6 of SELEX™ as described above are listed in Table 13 below. A plot of Round 6 sequences bound to target thrombin is shown in FIG. 9.

TABLE 13

Corresponding cDNAs of the Round 6 sequences of
dRmY SELEX ™ against thrombin.

SEQ ID No.221 Thrombin A1
GGGAGAGGAGAGAACGTTCTACGTGTGATGGGGTGAGAGGATGAGTTAGT
GACGCTGTCGATCGATCGATCGATG
SEQ ID No.222 Thrombin A2
GGGAGAGGAGAGAACGTTCTACAATGGGAGGGTAATAGTGATGAGGAGAG
GCGCTGTCGATCGATCGATCGATG
SEQ ID No.223 Thrombin A3
GGGAGAGGAGAGAACGTTCTACGGGTCGTGAGATAATGGCTCCCGTATTC
AGCGCTGTCGATCGATCGATCGATG
SEQ ID No.224 Thrombin A4
GGGAGAGGAGAGAACGTTCTACGGGTCGTGAGATAATGGCTCCCGTATTC
AGCGCTGTCGATCGATCGATCGATG
SEQ ID No.225 Thrombin B1
GGGAGAGGAGAGAACGTTCTACAGGTAGCGTGAGGGGGTGTTAATAGAGG
GGCGCTGTCGATCGATCGATCGATG
SEQ ID No.226 Thrombin B2
GGGAGAGGAGAGAACGTTCTACGATAGGATGGGTGGGACAGGAGAGGGAG
TGCGCTGTCGATCGATCGATCGATG
SEQ ID No.227 Thrornbin B3
GGGAGAGGAGAGAACGTTCTACCAGTGAGGGCAGTGTCAGATTGAGAGGA
GGCGCTGTCGATCGATCGATCGATG
SEQ ID No.228 Thrombin B4
GGGAGAGGAGAGAACGTTCTACCTTGCCTAACAGGAGGTGGAGTATTGGA
CCCGCTGTCGATCGATCGATCGATG
SEQ ID No.229 Thrombin C1
GGGAGAGGAGAGAACGTTCTACCTTGCCTAACAGGAGGTGGAGTATTGGA
CCCGCTGTCGATCGATCGATCGATG
SEQ ID No.230 Thrombin C2
GGGAGAGGAGAGAACGTTCTACGTCGTGAGTAATGGCTCGTAGATGAGGT
CGCTGTCGATCGATCGATCGATG
SEQ ID No.231 Throinbin C3
GGGAGAGGAGAGAACGTTCTACGGGATTAAGAGGGGAGAGGAGCAGTTGA
GCGCTGTCGATCGATCGATCGATG
SEQ ID No.232 Thrombin C4
GGGAGAGGAGAGAACGTTCTACTCCGGTTGGGGTATCAGGTCTACGGACT
GACGCTGTCGATCGATCGATCGATG
SEQ ID No.233 Thrombin D1
GGGAGAGGAGAGAACGTTCTACGGGTCGTGAGATAATGGCTCCCGTATTC
AGCGCTGTCGATCGATCGATCGATG
SEQ ID No.234 Thrombin D2
GGGAGAGGAGAGAACGTTCTACGGGTCGTGAGATAATGGCTCCCGTATTC
AGCGCTGTCGATCGATCGATCGATG
SEQ ID No.235 Thrombin D3
GGGAGAGGAGAGAACGTTCTACATGACAAGAGGGGGTTGTGTGGGATGGC
AGCGCTGTCGATCGATCGATCGATG
SEQ ID No.236 Thrombin D4
GGGAGAGGAGAGAACGTTCTACACAGGGAGGGGAGCGGAGAGGAGAGAGG
GTACGCTGTCGATCGATCGATCGATG
SEQ ID No.237 Thrombin E1
GGGAGAGGAGAGAACGTTCTACGGGTCGTGAGATAATGGCTCCCGTATTC
AGCGCTGTCGATCGATCGATCGATG
SEQ ID No.238 Thrombin E2
GGGAGAGGAGAGAACGTTCTACGTCGTGAGTAATGGCTCGTAGATGAGGT
CGCTGTCGATCGATCGATCGATG
SEQ ID No.239 Thrombin E4
GGGAGAGGAGAGAACGTTCTACGGGTCGTGAGATAATGGCTCCCGTATTC
AGCGCTGTCGATCGATCGATCGATG
SEQ ID No.240 Thrombin F1
GGGAGAGGAGAGAACGTTCTACGGGTCGTGAGATAATGGCTCCCGTATTC
AGCGCTGTCGATCGATCGATCGATG
SEQ ID No.241 Thrombin F2
GGGAGAGGAGAGAACGTTCTACCTTGCCTAACAGGAGGTGGAGTATTGGA
CCCGCTGTCGATCGATCGATCGATG
SEQ ID No.242 Thrombin F3
GGGAGAGGAGAGAACGTTCTACGGCTATGCGTCGTGAGTCAATGGCCCGC
ATCGCTGTCGATCGATCGATCGATG
SEQ ID No.243 Thrombin F4
GGGAGAGGAGAGAACGTTCTACGGGTCGTGAGATAGTGGCTCCCGTATTC
AGCGCTGTCGATCGATCGATCGATG
SEQ ID No.244 Thrombin G1
GGGAGAGGAGAGAACGTTCTACGGGTCGTGAGATAATGGCTCCCGTATTC
AGCGCTGTCGATCGATCGATCGATG
SEQ ID No.245 Thrombin G2
GGGAGAGGAGAGAACGTTCTACGGGTCGTGAGATAATGGCTCCCGTATTC
AGCGCTGTCGATCGATCGATCGATG
SEQ ID No.246 Thrombin G3
GGGAGAGGAGAGAACGTTCTACCTTGTCTAACAGGAGGTGGAGTATTGGA
CCCGCTGTCGATCGATCGATCGATG
SEQ ID No.247 Thrombin G4
GGGAGAGGAGAGAACGTTCTACGACTTTGAGGGTGGTGAGAGTGGAAGAG
AGCGCTGTCGATCGATCGATCGATG
SEQ ID No.248 Thrombin H1
GGGAGAGGAGAGAACGTTCTACGGTAGGGTATGACCAGGGAGGTATTGGA
GGCGCTGTCGATCGATCGATCGATG
SEQ ID No.249 Thrombin H2
GGGAGAGGAGAGAACGTTCTACGGGTCGTGAGATAATGGCTCCCGTATTC
AGCGCTGTCGATCGATCGATCGATG
SEQ ID No.250 Thrombin H3
GGGAGAGGAGAGAACGTTCTACGGGTCGTGAGATAATGGCTCCCGTATTC
AGCGCTGTCGATCGATCGATCGATG
SEQ ID No.251 Thrombin H4
GGGAGAGGAGAGAACGTTCTACGTTATGCATGTGGAGAGTGAGAGAGGGC
GCTGTCGATCGATCGATCGATG

### Example 5

### dRmY SELEX™ of Aptamers Against VEGF

While fully 2′-OMe substituted oligonucleotides are the most stable modified aptamers, substituting the purines with deoxy purine nucleotides also results in stable transcripts. When dRmY (deoxy purines, A and G, and 2′-OMe pyrimidines) transcription conditions are used, the products are very DNase-resistant and useful as stable therapeutics. This result is surprising since the composition of the dRmY transcripts is approximately 50% DNA RNA, which is notoriously easily degraded by nucleases. Also, when dRmY transcription conditions are used, there is no requirement for a 2′-OH GTP spike. Libraries of transcription templates were used to generate pools of oligonucleotides incorporating 2′-O-methylpyrimidine NTPs (U and C) and deoxy purines (A and G) NTPs under various transcription conditions. The transcription template (ARC256) and the transcription conditions are described below as dRmY.

| | |
|---|---|
| ARC2S6: DNA transcription template | |
| 5′-CATCGATCGATCGATCGACAGCGNNNNNNNN | (SEQ ID NO:453) |
| NNNNNNNNNNNNNNNNNNNNNNGTAGAACGTTCT | |
| CTCCTCTCCCTATAGTGAGTCGTATTA-3′ | |

ARC256 dRmY transcription product is:

| | |
|---|---|
| 5′-GGGAGAGGAGAGAACGUUCUACNNNNNNNNN | (SEQ ID NO:464) |
| NNNNNNNNNNNNNNNNNNNNNCGCUGUCGAUCGA | |
| UCGAUCGAUG-3′ | |

When deoxy purines, A and G, and 2′-OMe pyrimidines (dRmY) conditions were used, the reaction conditions were 1×Tc buffer, 50-300 nM double stranded template (300 nm template was used for round 1, and for subsequent rounds a 1/10 dilution of an optimized PCR reaction, using conditions described herein, was used), 9.6 mM MgCl₂, 2.9 mM MnCl₂, 2 mM each base, 30 μM GTP, 2 mM Spermine, 10% PEG-8000, 0.25 units inorganic pyrophosphatase, and 1.5 units Y639F single mutant RNA polymerase.

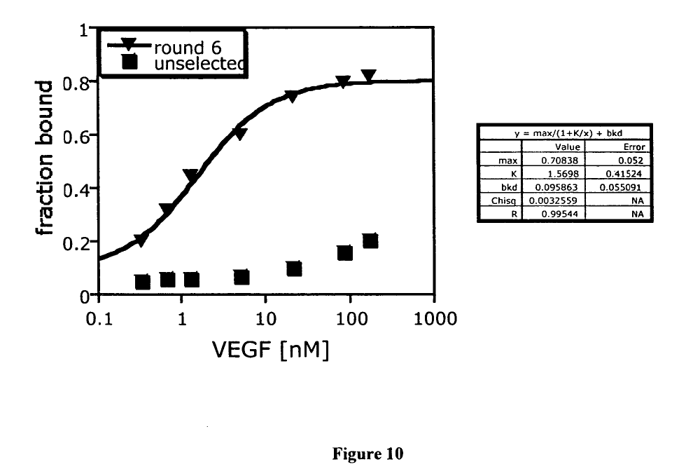

These pools were then used in SELEX™ to select for aptamers against VEGF as a target. The sequences obtained after round 6 of SELEX™ as described above are listed in an alignment show in Table 14 below. A plot of Round 6 sequences bound to target VEGF is shown in FIG. 10.

TABLE 14

Corresponding cDNAs of the Round 6 sequences of
dRmY SELEX ™ against VEGF.

SEQ ID No.252 VEGF A9
GGGAGAGGAGAGAACGTTCTACCATGTCTGCGGGAGGTGAGTAGTGATCC
TGCGCTGTCGATCGATCGATCGATG
SEQ ID No.253 VEGF A10
GGGAGAGGAGAGAACGTTCTACAGAGTGGGAGGGATGTGTGACACAGGTA
GGCGCTGTCGATCGATCGATCGATG
SEQ ID No.254 VEGF A11
GGGAGAGGAGAGAACGTTCTACGCTCCATGACAGTGAGGTGAGTAGTGAT
CGCTGTCGATCGATCGATCGATG
SEQ ID No.255 VEGF A12
GGGAGAGGAGAGAACGTTCT CGATGCTGACAGGGTGTGTTCAGTAATGG
CTCGCTGTCGATCGATCGATCGATG
SEQ ID No.256 VEGF B9
GGGAGAGGAGAGAACGTTCTACCAGCAAACAGGGTCAGGTGAGTAGTGAT
GACGCTGTCGATCGATCGATCGATG
SEQ ID No.257 VEGF B10
GGGAGAGGAGAGAACGTTCTACGACAAGCCGGGGGTGTTCAGTAGTGGCA
ACCGCTGTCGATCGATCGATCGATG
SEQ ID No.258 VEGF B11
GGGAGAGGAGAGAACGTTCTACATATGGCGCTGGAGGTGAGTAATGATCG
TGCGCTGTCGATCGATCGATCGATG
SEQ ID No.259 VEGF B12
GGGAGAGGAGAGAACGTTCTACGGGGCGATAGCGTTCAGTAGTGGCGCCG
GTCGCTGTCGATCGATCGATCGATG
SEQ ID No.260 VEGF C9
GGGAGAGGAGAGAACGTTCTACATAGCGGACTGGGTGCATGGAGCGGCGC
ACGCTGTCGATCGATCGATCGATG
SEQ ID No.261 VEGF C10
GGGAGAGGAGAGAACGTTCTACGGGTCAACAGGGGCGTTCAGTAGTGGCG
GCGCTGTCGATCGATCGATCGATG
SEQ ID No.262 VEGF C11
GGGAGAGGAGAGAACGTTCTACGCATGCGAGCTGAGGTGAGTAGTGATCA
GTCGCTGTCGATCGATCGATCGATG
SEQ ID No.263 VEGF C12
GGGAGAGGAGAGAACGTTCTACATGCGACAGGGGAGTGTTCAGTAGTGGC
ACGCTGTCGATCGATCGATCGATG
SEQ ID No.264 VEGF D9
GGGAGAGGAGAGAACGTTCTACCCCATCGTATGGAGTGCGGAACGGGGCA
TACGCTGTCGATCGATCGATCGATG
SEQ ID No.265 VEGF D10
GGGAGAGGAGAGAACGTTCTACAGTGAGGCGGGAGCGTTTCAGTAATGGC
GCTGTCGATCGATCGATCGATG
SEQ ID No.266 VEGF D12
GGGAGAGGAGAGAACGTTCTACACAGCGTCGGGTGTTCAGTAATGGCGCA
GCGCTGTCGATCGATCGATCGATG
SEQ ID No.267 VEGF E9
GGGAGAGGAGAGAACGTTCTACGGTGTTCAGTAGTGGCACAGGAGGAAGG
GATGCTGTCGATCGATCGATCGATG
SEQ ID No.268 VEGF E10
GGGAGAGGAGAGAACGTTCTACAGTTCAGGCGTTAGGCATGGGTGTCGCT
TTCGCTGTCGATCGATCGATCGATG
SEQ ID No.269 VEGF E11
GGGAGAGGAGAGAACGTTCTACATGCGACATGCGAGTGTTCAGTAGCGGC
AGCGCTGTCGATCGATCGATCGATG
SEQ ID No.270 VEGF E12
GGGAGAGGAGAGAACGTTCTACCTATGGCGTTACAGCGAGGTGAGTAGTG
ATCGCTGTCGATCGATCGATCGATG
SEQ ID No.271 VEGF F9
GGGAGAGGAGAGAACGTTCTACCAGCCGATCCAGCCAGGCGTTCAGTAGT
GGCGCTGTCGATCGATCGATCGATG
SEQ ID No.272 VEGF F10
GGGAGAGGAGAGAACGTTCTACGGCACAGGCACGGCGAGGTGAGTAATGA
TCGCTGTCGATCGATCGATCGATG
SEQ ID No.273 VEGF G9
GGGAGAGGAGAGAACGTTCTACTGTGGACAGCGGGAGTGCGGAACGGGGT
CGCTGTCGATCGATCGATCGATG
SEQ ID No.274 VEGF G10
GGGAGAGGAGAGAACGTTCTACTGATGCTGCGAGTGCATGGGGCAGGCGC
TTCGCTGTCGATCGATCGATCGATG
SEQ ID No.275 VEGF G11
GGGAGAGGAGAGAACGTTCTACGGTACAATGGGAATGACAGTGATGGGTA
GCCGCTGTCGATCGATCGATCGATG
SEQ ID No.276 VEGF G12
GGGAGAGGAGAGAACGTTCTACATGGACAGCGAAGCATGGGGGAGGCGCA
CGCTGTCGATCGATCGATCGATG
SEQ ID No.277 VEGF H9
GGGAGAGGAGAGAACGTTCTACTGGGAGCGACAGTGAGCATGGGGTAGGC
GCCGCTGTCGATCGATCGATCGATG
SEQ ID No.278 VEGF H11
GGGAGAGGAGAGAACGTTCTACCGGCGAGCAGGTGTTCAGTAGTGGCTTT
GCGCTGTCGATCGATCGATCGATG
SEQ ID No.279 VEGF H12
GGGAGAGGAGAGAACGTTCTACGATCAGTGAGGGAGTGCAGTAGTGGCTC
GTCGCTGTCGATCGATCGATCGATG

### Example 6

### Plasma Stability of 2′-OMe NTPs (mN) and dRmY Oligonucleotides

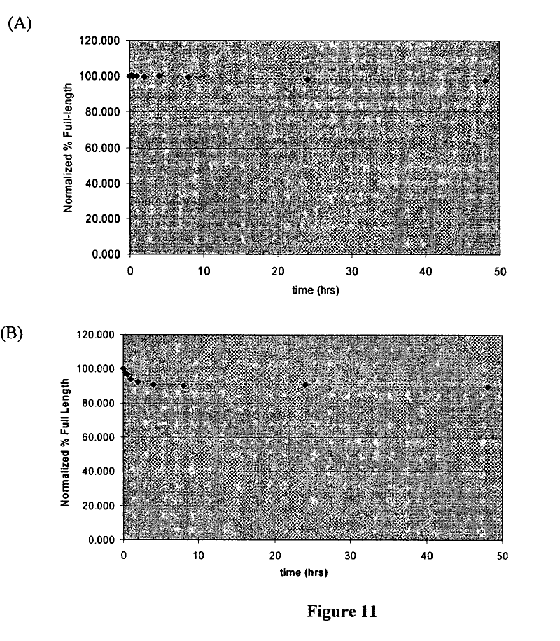

An oligonucleotide of two sequences linked by a polyethylene glycol polymer (PEG) was synthesized in two versions: (1) with all 2′-OMe NTPs (mN): 5′-GGAGCAGCACC-3′ (SEQ ID NO:457)-[PEG]-GGUGCCAAGUCGUUGCUCC-3′ (SEQ ID NO:458) and (2) with 2′-OH purine NTPs and 2′-OMe pyrimidines (dRmY) GGAGCAGCACC-3′ (SEQ ID NO:465)-[PEG]-GGUGCCAAGUCGUUGCUCC-3′ (SEQ ID NO:466). These oligonucleotides were evaluated for full length stability. FIG. 11A shows a degradation plot of the all 2′-OMe oligonucleotide with 3′idT and FIG. 11B shows a degradation plot of the dRmY oligonucleotide. The oligonucleotides were incubated at 50 nM in 95% rat plasma at 37° C. and show a plasma half-life of much greater than 48 hours for each, and that they have very similar plasma stability profiles.

### Example 7

### rRmY and rGmH 2′-OMe SELEX™ Against Human IL-23

Selections were performed to identify aptamers containing 2′-OMe C, U and 2′-OH G, A (rRmY), and 2′-O-Methyl A, C, and U and 2′-OH G (rGmH). All selections were direct selections against human IL-23 protein target which had been immobilized on a hydrophobic plate. Selections yielded pools significantly enriched for h-IL-23 binding versus naïve, unselected pool. Individual clone sequences for h-IL-23 are reported herein, but h-IL-23 binding data for the individual clones are not shown.

Pool Preparation. A DNA template with the sequence 5′-GGGAGAGGAGAGAACGTTCTACNNNNNNNNNNNNNNNNNNNNNNNNNNNNNNCG CTGTCGATCGATCGATCGATG-3′ (SEQ ID NO:459) was synthesized using an ABI EXPEDITE™ DNA synthesizer, and deprotected by standard methods. The templates were amplified with the primers PB.118.95.G: 5′-GGGAGAGGAGAGAACGTTCTAC-3′ (SEQ ID NO:460) and STC.104.102.A (5′-CATCGATCGATCGATCGACAGC-3′ (SEQ ID NO:461) and then used as a template (200 nm template was used for round 1, and for subsequent rounds approximately 50 nM, a 1/10 dilution of an optimized PCR reaction, using conditions described herein, was used) for in vitro transcription with Y639F single mutant T7 RNA polymerase. Transcriptions were done using 200 mM HEPES, 40 mM DTT, 2 mM spermidine, 0.01% TritonX-100, 10% PEG-8000, 5 mM MgCl₂, 1.5 mM MnCl₂, 500 μM NTPs, 500 μM GMP, 0.01 units/μl inorganic pyrophosphatase, and Y639F single mutant T7 polymerase. Two different compositions were transcribed rRmY and rGmH.

Selection. Each round of selection was initiated by immobilizing 20 pmoles of h-IL-23 to the surface Nunc Maxisorp hydrophobic plates for 2 hours at room temperature in 100 μL of 1×Dulbecco's PBS. The supernatant was then removed and the wells were washed 4 times with 120 μL wash buffer (1×DPBS, 0.2% BSA, and 0.05% Tween-20). Pool RNA was heated to 90° C. for 3 minutes and cooled to room temperature for 10 minutes to refold. In round 1, a positive selection step was conducted. Briefly, 1×10¹⁴ molecules (0.2 nmoles) of pool RNA were incubated in 100 μL binding buffer (1×DPBS and 0.05% Tween-20) in the wells with immobilized protein target for 1 hour. The supernatant was then removed and the wells were washed 4× with 120 μL wash buffer. In subsequent rounds a negative selection step was included. The pool RNA was also incubated for 30 minutes at room temperature in empty wells to remove any plastic binding sequences from the pool before the positive selection step. The number of washes was increased after round 4 to increase stringency. In all cases, the pool RNA bound to immobilized h-IL-23 was reverse transcribed directly in the selection plate after by the addition of RT mix (3′ primer, STC.104.102.A, and Thermoscript RT, Invitrogen) followed by incubated at 65° C. for 1 hour. The resulting cDNA was used as a template for PCR (Taq polymerase, New England Biolabs) “Hot start” PCR conditions coupled with a 60° C. annealing temperature were used to minimize primer-dimer formation. Amplified pool template DNA was desalted with a Centrisep column according to the manufacturer's recommended conditions and used to program transcription of the pool RNA for the next round of selection. The transcribed pool was gel purified on a 10% polyacrylamide gel every round. Table 15 shows the RNA pool concentrations used per round of selection.

TABLE 15
RNA pool concentrations per round of selection.

| pmoles | | | | | | | | |
|---|---|---|---|---|---|---|---|---|
| Pool | rRmY | | | PD- | rGmH | | | |
| used | 2OMe | | | GF- | 3OMe | | | PDGF- |
| Round | IL23 | hIgE | mIgE | BB | IL23 | hIgE | mIgE | BB |
| 1 | 200 | 200 | 200 | 200 | 200 | 200 | 200 | 200 |
| 2 | 110 | 140 | 130 | 135 | 40 | 50 | 40 | 60 |
| 3 | 65 | 115 | 60 | 160 | 100 | 190 | 90 | 160 |
| 4 | 50 | 40 | 40 | 30 | 170 | 120 | 40 | 240 |
| 5 | 80 | 130 | 130 | 110 | 100 | 60 | 40 | 70 |
| 6 | 100 | 80 | 90 | 39 | 110 | 140 | 90 | 90 |
| 7 | 50 | 90 | 130 | 170 | 70 | 80 | 130 | 90 |
| 8 | 120 | | 190 | 150 | 60 | 90 | 110 | 130 |
| 9 | 120 | | 210 | 170 | 80 | 80 | 100 | 100 |
| 10 | 130 | | 210 | 180 | | | | |
| 11 | 110 | | | 210 | | | | |

The selection progress was monitored using a sandwich filter binding assay. The 5′-⁼P-labeled pool RNA was refolded at 90° C. for 3 minutes and cooled to room temperature for 10 minutes. Next, pool RNA (trace concentration) was incubated with h-IL-23 DPBS plus 0.1 mg/ml tRNA for 30 minutes at room temperature and then applied to a nitrocellulose and nylon filter sandwich in a dot blot apparatus (Schleicher and Schuell). The percentage of pool RNA bound to the nitrocellulose was calculated and monitored approximately every 3 rounds with a signal point screen (+/−250 nM h-IL-23). Pool K_{D} measurements were measured using a titration of protein and the dot blot apparatus as described above.

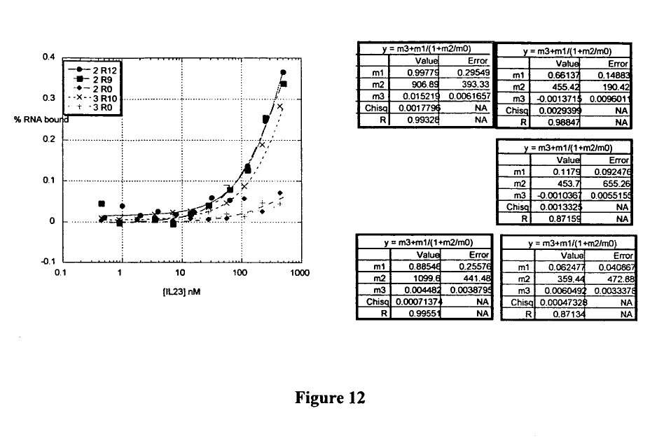

Selection. The rRmY h-IL-23 selection was enriched for h-IL-23 binding vs. the naïve pools after 4 rounds of selection. The selection stringency was increased and the selection was continued for 8 more rounds. At round 9 the pool K_{D} was approximately 500 nM or higher. The rGmH selection was enriched over the naïve pool binding at round 10. The pool K_{D} is also approximately 500 nM or higher. The pools were cloned using TOPO TA cloning kit (Invitrogen) and individual sequences were generated. FIG. 12 shows pool binding data to h-IL-23 for the rGmH round 10 and rRmY round 12 pools. Dissociation constants were estimated fitting data to the equation: fraction RNA bound=amplitude*K_{D}/(K_{D}+[h-IL-23]). Table 16 shows the individual clone sequences for round 12 of the rRmY selection. There is one group of 6 duplicate sequences and 4 pairs of 2 duplicate sequences out of 48 clones. All 48 clones will be labeled and tested for binding to 200 mM h-IL-23. Table 17 shows the individual clone sequences for round 10 of the rGmH selection. Binding data is shown in FIG. 14.

TABLE 16

Corresponding cDNAs of the Individual Clone
Sequences for Round 12 of the rRmY Selection.

SEQ ID No.280 ARX34P2.G01
GGGAGAGGAGAGAACGTTCTACAAATGAGAGCAGGCCGAAAAGGAGTCGC
TCGCTGTCGATCGATCGATCGATGAAGGGCG
SEQ ID No.281 ARX34P2.A06
GGGAGAGGAGAGAACGTTCTACAAAGGATCAATCTTTCGGCGTATGTGTG
AGCGCTGTCGATCGATCGATCGATGAAGGGCG
SEQ ID No.282 ARX34P2.E02
GGGAGAGGAGAGAACGTTCTACGGTAAAGCAGGCTGACTGAAAGGTTGAA
GTCGCTGTCGATCGATCGATCGATGAAGGGCG
SEQ ID No.283 ARX34P2.H05
GGGAGAGGAGAGAACGTTCTACAGGTTAAAGCAGGCTCAGGAATGGAAGT
CGCTGTCGATCGATCGATCGATGAAGGGCG
SEQ ID No.284 ARX34P2.G04
GGGAGAGGAGAGAACGTTCTACCAAAGCAGGCTCATAGTAATATGGAAGT
CGCTGTCGATCGATCGATCGATGAAGGGCG
SEQ ID No.285 ARX34P2.G03
GGGAGAGGAGAGAACGTTCTACAAAAGAGAGCAGGCCGAAAAGGAGTCGC
TCGCTGTCGATCGATCGATCGATGAAGGGCG
SEQ ID No.286 ARX34P2.N06
GGGAGAGGAGAGAACGTTCTACAAAAGGCAGGCTCAGGGGATCACTGGAA
GTCGCTGTCGATCGATCGATCGATGAAGGGCG
SEQ ID No.287 ARX34P2.B01
GGGAGAGGAGAGAACGTTCTACAAAAAGCAGGCCGTATGGATATAAGGGA
GTCGCTGTCGATCGATCGATCGATGAAGGGCG
SEQ ID No.288 ARX34P2.B03
GGGAGAGGAGAGAACGTTCTACAAGTGCAGGCTGCAGACATATGCGAAGT
CGCTGTCGATCGATCGATCGATGAAGGGCG
SEQ ID No.289 ARX34P2.D05
GGGAGAGGAGAGAACGTTCTACAAAGGAGAGCAGGCCGAAAAGGAGTCGC
TCGCTGTCGATCGATCGATCGATGAAGGGCG
SEQ ID No.290 ARX34P2.C05
GGGAGAGGAGAGAACGTTCTACAAGATATAATTAAGGATAAGTGCAAAGG
AGACGCTGTCGATCGATCGATCGATGAAGGGCG
SEQ ID No.291 ARX34P2.C04
GGGAGAGGAGAGAACGTTCTACAGACAACAGCNAGAGGGAATCNCANACA
AAGACGCTGTCGATCGATCGATCGATGAAGGGCG
SEQ ID No.292 ARX34P2.E06
GGGAGAGAGAGAACGTTCTACAGATTCTAAGCGCAGGAATAAGTCACCAG
ACGCTGTCGATCGATCGATCGATGAAGGGCG
SEQ ID No.293 ARX34P2.A01
GGGAGAGGAGAGAACGTTCTACGAAAATGAGCATGGAAGTGGGAGTACGT
GCCGCTGTCGATCGATCGATCGATGAAGGGCG
SEQ ID No.294 ARX34P2.C06
GGGAGAGGAGAGAACGTTCTACGAAGAGGCGCCGGAAGTGAGAGTAAGTG
CGCTGTCGATCGATCGATCGATGAAGGGCG
SEQ ID No.295 ARX34P2.E04
GGGAGAGGAGAGAACGTTCTACGAAGTGAGTTTCCGAAGTGAGAGTACGA
ACGCTGTCGATCGATCGATCGATGAAGGGCG
SEQ ID No.296 ARX34P2.E04
GGGAGAGGAGAGAACGTTCTACGAATGAGAGCAGGCCGAAAAGGAGTCGC
TCGCTGTCGATCGATCGATCGATGAAGGGCG
SEQ ID No.297 ARX34P2.E04
GGGAGAGGAGAGAACGTTCTACGAGAGGCAAGAGAGAGTCGCATAAAAAA
GACGCTGTCGATCGATCGATCGATGAAGGGCG
SEQ ID No.298 ARX34P2.D06
GGGAGAGGAGAGAACGTTCTACGCAGGCTGTCGTAGACAAACGATGAAGT
CGCGCTGTCGATCGATCGATCGATGAAGGGCG
SEQ ID No.299 ARX34P2.F05
GGGAGAGGAGAGAACGTTCTACGGAAAAAGATATGAAAGAAAGGATTAAG
AGACGCTGTCGATCGATCGATCGATGAAGGGCG
SEQ ID No.300 ARX34P2.H02
GGGAGAGGAGAGAACGTTCTACGGAAGGNAACAANAGCACTGTTTGTGCA
GGCGCTGTCGATCNATCNATCNATGAAGGGCG
SEQ ID No.301 ARX34P2.C03
GGGAGAGGAGAGAACGTTCTACGGAGCATANGGCNTGAAACTGAGANAGT
AACGCTGTCGATCGATCGATCGATGAAGGGCG
SEQ ID No.302 ARX34P2.D01
GGGAGAGGAGAGAACGTTCTACGAAAAAGGATATGAGAGAAAGGATTAAG
AGACGCTGTCGATCGATCGATCGATGAAGGGCG
SEQ ID No.303 ARX34P2.A03
GGGAGAGGAGAGAACGTTCTACATACATAGGCGCCGCGAATGGGAAAGAA
AGCGCTGTCGATCGATCGATCGATGAAGGGCG
SEQ ID No.304 ARX34P2.B02
GGGAGAGGAGAGAACGTTCTACTCATGAAGCCATGGTTGTAATTCTGTTT
GGCGCTGTCGATCGATCGATCGATGAAGGGCG
SEQ ID No.305 ARX34P2.C01
GGGAGAGGAGAGAACGTTCTACTAATGCAGGCTCAGTTACTACTGGAAGT
CGCTGTCGATCGATCGATCGATGAAGGGCG
SEQ ID No.306 ARX34P2.D07
GGGAGAGGAGAGAACGTTCTACTTTCATAGGCGGGATTATGGAGGAGTAT
TCGCTGTCGATCGATCGATCGATGAAGGGCG
SEQ ID No.307 ARX34P2.G0S
AGGAGAGGAGAGAACGTTCTACTAGAAGCAGGCTCGAATACAATTCGGAA
GTCGCTGTCGATCGATCGATCGATGAAGGGCG
SEQ ID No.308 ARX34P2.F06
GGGAGAGGAGAGAACGTTCTACTTAGCGATGTCGGAAGAGAGAGTACGAG
GACGCTGTCGATCGATCGATCGATGAAGGGCG
SEQ ID No.309 ARX34P2.F02
GGGAGAGGAGAGAACGTTCTACTTGCGAAGACCGTGGAAGAGGAGTACTG
GTCGCTGTCGATCGATCGATCGATGAAGGGCG
SEQ ID No.310 ARX34P2.E05
GGGAGAGGAGAGAACGTTCTACTTTTGGTGAAGGTGTAAGAGTGGCACTA
CACGCTGTCGATCGATCGATCGATGAAGGGCG
SEQ ID No.311 ARX34P2.A05
GGGAGAGGAGAGAACGTTCTACCATCAGTTGTGGCGATTATGTGGGAGTA
TGCGCTGTCGATCGATCGATCGATGAAGGGCG
SEQ ID No.312 ARX34P2.E03
GGGAGAGGAGAGAACGTTCTACANAANAACATGCGATTAAAGATCATGAA
CAGCGCTGTCGATCGATCGATCGATGAAGGGCG
SEQ ID No.313 ARX34P2.F04
GGGAGAGGAGAGAACGTTCTACATAAGCAGGCTCCGATAGTATTCGGGAA
GTCGCTGTCGATCGATCGATCGATGAAGGGCG

TABLE 17

Corresponding cDNAs of the Individual Clone
Sequences for Round 10 of the rGmH Selection.

SEQ ID No.314 ARX34P2.E10
GGGAGAGGAGAGAACGTTCTACTTTCGGAATGCGATGGGGGTGATTCGTG
GTCGCTGTCGATCGATCGATCGATGAAGGGCG
SEQ ID No.315 ARX34P2.H09
GGGAGAGGAGAGAACGTTCTACCTGTTGAGGCTAAGTGGATGATTGAGGG
CGCTGTCGATCGATCGATCGATGAAGGGCG
SEQ ID No.316 ARX34P2.A07
GGGAGAGGAGAGAACGTTCTACCTGGGTCGGTGCGATTGGAGATGTCGTT
GCGCTGTCGATCGATCGATCGATGAAGGGCG
SEQ ID No.317 ARX34P2.A12
GGGAGAGGAGAGAACGTTCTACCTGATGTCAGGTTGTTTGGAGATTATCT
GACNCTGTCNATCGATCGATCGATGAAGGGCG
SEQ ID No.318 ARX34P2.A08
GGGAGAGGAGAGAACGTTCTACCTCGCGCGACGAGCGAATTTCCGGATGC
GGCGCTGTCGATCGATCGATCGATGAAGGGCG
SEQ ID No.319 ARX34P2.D12
GGGAGAGGAGAGAACGTTCTACCATGAATGATTGCGATCGTTGTTCGTGT
GGCGCTGTCGATCGATCGATCGATGAAGGGCG
SEQ ID No.320 ARX34P2.E11
GGGAGAGGAGAGAACGTTCTACTCCGACCACGCCTGGGTGATTCCTACNA
CGACGCTGTCGATCGATCGATCGATGAAGGGCG
SEQ ID No.321 ARX34P2.E12
GGGAGAGGAGAGAACGTTCTACTACTTTTGGGGATTCACTCCGCGCTGAT
GTCGCTGTCGATCGATCGATCGATGAAGGGCG
SEQ ID No.322 ARX34P2.D08
GGGAGAGGAGANAACGTTCTANTAGTGCTTGCGAGATAGTGTAGGATTAT
ACTGCTGTCGATCGATCGATCGATGAAGGGCG
SEQ ID No.323 ARX34P2.F07
GGGAGAGGAGAGAACGTTCTACTAGTGTCCTTCTCCACGTGGTTGTAATT
GTCGCTGTCGATCGATCGATCGATGAAGGGCG
SEQ ID No.324 ARX34P2.B11
GGGAGAGGAGAGAACGTTCTACTATTGTGGCGCTTGTTGGACTAACTGAC
TACGCTGTCGATCGATCGATCGATGAAGGGCG
SEQ ID No.325 ARX34P2.F12
GGGAGAGGAGAGAACGTCCTACTTCGATTGTGATCTTGTGGCGGCCTGTG
AGCGCTGTCGATCGATCGATCGATGAAGGGCG
SEQ ID No.326 ARX34P2.A09
GGGAGAGGAGAGAACGTTCTACTTGGCGATGTCGGAAGAGAGAGTACGAG
GGCGCTGTCGATCGATCGATCGATGAAGGGCG
SEQ ID No.467 ARX34P2.B07
GGGAGAGGAGAGAACGTTCTACTTGCTGTGACGGACGGGCTTGAGAGGCT
CGCTGTCGATCGATCGATCGATGAAGGGCG
SEQ ID No.327 ARX34P2.D07
GGGAGAGGAGAGAACGTTCTACTTGAANCTGCGTGAATTGANAGTAACGA
AGCGCTGTCAATCGATCNATCAATNAAGGGCG
SEQ ID No.328 ARX34P2.H10
GGGAGAGGAGAGAACGTTCTACTCGAGAGGACATGTGGATCCGGTTCGCG
TGCGCTGTCGATCGATCGATCGATGAAGGGCG
SEQ ID No.329 ARX34P2.H07
GGGAGAGGAGAGAACGTTCTACTGTGATGCGGTTTGCGTCGACCGGATTC
GTCGCTGTCGATCGATCGATCGATGAAGGGCG
SEQ ID No.330 ARX34P2.F11
GGGAGAGGAGAGAACGTTCTACTGTGTGATTGGGCGCATGTCGAGGCGAC
ACGCTGTCGATCGATCGATCGATGAAGGGCG
SEQ ID No.331 ARX34P2.C07
GGGAGAGGAGAGAACGTTCTACTGATTAAGATGCGCTGGTAGAGCGGTGG
GCGCTGTCGATCGATCGATCGATGAAGGGCG
SEQ ID No.332 ARX34P2.A10
GGGAGAGGAGAGAACGTTCTACTGGTTAATTTGCATGCGCGANTAACNTG
NTCGCTGTCGATCGATCGATCGATGAAGGGCG
SEQ ID No.333 ARX34P2.G10
GGGAGAGGAGAGAACGTTCTACTGGGAAGCGGTAACTTGGATTCACCGAT
CCGCTGTCGATCGATCGATCGATGAAGGGCG
SEQ ID No.334 ARX34P2.H11
GGGAGAGGAGAGAACGTTCTACTGTTACGGAGATGATGGGTTTGGCTGTT
GGCGCTGTCGATCGATCGATCGATGAAGGGCG
SEQ ID No.335 ARX34P2.C07
GGGAGAGGAGAGAACGTTCTACTTGTGGACTGAGATACGATTCGGAGCTG
GCGCTGTCGATCGATGATCGATGAAGGGCG
SEQ ID No.336 AR134P2.E08
GGGAGAGGAGAGACGTTCTACTTGTGAGTTTCCTTGGGCCTTGAGCGTGG
GCGCTGTCGATCGATCGATCGATGAAGGGCG
SEQ ID No.337 ARX34P2.A11
GGGAGAGGAGAGAACGTTCTACAGGTGATGTGAGCCGATTGTGAAGTTTT
GTCGCTGTCGATCGATCGATCGATGAAGGGCG
SEQ ID No.338 ARX34P2.B08
GGGAGAGGAGAGAACGTTCTACAGCGGATGTTTGGGGGTGTGTGTTGGTT
GTCGCTGTCGATCGATCGATCGATGAAGGGCG
SEQ ID No.339 ARX34P2.B09
GGGAGAGGAGAGAACGTTCTACATGCGGTGGTGGTCTTCGATGGGTGGAA
GTCGCTGTCGATCGATCGATCGATGAAGGGCG
SEQ ID No.340 ARX34P2.B12
GGGAGAGGAGAGAACGTTCTACATTGGAGGGGCGCATGTGGTCTGTTTGA
TGCGCTGTCGATCGATCGATCGATGAAGGGCG
SEQ ID No.341 ARX34P2.P10
GGGAGAGGAGAGAACGTTCTACGTGTTTCGCGGATTTGAAGAGGAGTAAA
ATCGCTGTCGATCGATCGATCGATGAAGGGCG
SEQ ID No.342 ARX34P2.E10
GGGAGAGGAGAGAACGTTCTACGTGTGCGTGTTCGGGAAGGGAGAGTGCC
GAGGCTGTCGATCGATCGATCGATGAAGGGCG
SEQ ID No.343 ARX34P2.B10
GGGAGAGGAGAGAACGTTCTACGTGTGTGGTGTGCGATGCTTGGCTGTTT
GTCGCTGTCGATCGATCGATCGATGAAGGGCG
SEQ ID No.344 ARX34P2.C08
GGGAGAGGAGAGAACGTTCTACGGTTTGTGTGGCTTGGATCTGAAGACTA
AGCGCTGTCGATCGATCGATCGATGAAGGGCG
SEQ ID No.345 ARX34P2.F09
GGGAGAGGAGAGAACGTTCTACGGTTCTGGGCTTGTGTGTGAGGATTGAC
GGCGCTGTCGATCGATCGATCGATGAAGGGCG
SEQ ID No.346 ARX34P2.C10
GGGAGAGGAGAGAACGTTCTACGATGATGAAGGCGAAAAGACGAGGCTGT
CGATCGATCGATCGATGAAGGGCG
SEQ ID No.347 ARX34P2.C11
GGGAGAGGAGAGAACGTTCTACGAGTGCTGATGCGTGTCCTGGGATGGAA
TTCGCTGTCGATCGATCGATCGATGAAGGGCG
SEQ ID No.348 ARX34P2.D09
GGGAGAGGAGAGAACGTTCTACGCGTTTATAGCGATCGATGATGATATAG
GCCGCTGTCGATCGATCGATCGATGAAGGGCG
SEQ ID No.349 ARX34P2.D10
GGGAGAGGAGAGAACGTTCTACGCGTTCAAATGGGATAGAATTGGCTGCG
GGCGCTGTCGATCGATCGATCGATGAAGGGCG
SEQ ID No.350 ARX34P2.D11
GGGAGAGGAGAGAACGTTCTACGAAATTGTGCGTCAGTGTGAGGCGGTTT
GCTGTCGATCGATCGATCGATGAAGGGCG
SEQ ID No.351 ARX34P2.E07
GGGAGAGGAGAGAACGTTCTACGGTCGAAATGAGGGTCTGGAGTTCCGAC
GACGCTGTCGATCGATCGATCGATGAAGGGCG
SEQ ID No.352 ARX34P2.E09
GGGAGAGGAGAGAACGTTCTACGAATTTGGTAATCTGGGTGACTTAGGAT
GTCGCTGTCGATCGATCGATCGATGAAGGGCG
SEQ ID No.353 ARX34P2.G12
GGGAGAGGAGAGAACGTTCTACGATTTTTTGTGCCGAAGTAAGAGTACGC
GCGCTGTCGATCGATCGATCGATGAAGGGCG
SEQ ID No.354 ARX34P2.H08
AGGAGAGGAGAGAACGTTCTACGGAGTGTGCGCGGATGAAAACAGAAGTT
GTCGCTGTCNATCGATCNATCAATGAAGGGCG

### Example 8

### rRmY 2′-OMe SELEX™ Against Human IgE

Selections were performed to identify aptamers containing 2′-OMe C, U and 2′-OH G, A (rRmY). All selections were direct selections against human IgE protein target which had been immobilized on a hydrophobic plate. Selections yielded pools significantly enriched for h-IgE binding versus naive, unselected pool. Individual clone sequences for h-IgE are reported herein, but h-IgE binding data for the individual clones are not shown.

Pool Preparation. A DNA template with the sequence 5′-GGGAGAGGAGAGAACGTTCTACNNNNNNNNNNNNNNNNNNNNNNNNNNNNCG CTGTCGATCGATCGATCGATG-3′ (SEQ ID NO:459) was synthesized using an ABI EXPEDITE™ DNA synthesizer, and deprotected by standard methods. The templates were amplified with the primers PB.118.95.G 5′-GGGAGAGGAGAGAACGTTCTAC-3′(SEQ ID NO:460) and STC.104.102.A 5′-CATCGATCGATCGATCGACAGC-3′(SEQ ID NO:461) and then used as a template (200 nm template was used for round 1, and for subsequent rounds approximately 50 nM, a 1/10 dilution of an optimized PCR reaction, using conditions described herein, was used) for in vitro transcription with Y639F single mutant T7 RNA polymerase. Transcriptions were done using 200 mM HEPES, 40 mM DTT, 2 mM spermidine, 0.01% TritonX-100, 10% PEG-8000, 5 mM MgCl₂, 1.5 mM MnCl₂, 500 μM NTPs, 500 μM GMP, 0.01 units/μl inorganic pyrophosphatase, and Y639F single mutant T7 polymerase. Selection. Each round of selection was initiated by immobilizing 20 pmoles of h-IgE to the surface Nunc Maxisorp hydrophobic plates for 2 hours at room temperature in 100 μL of 1×Dulbecco's PBS. The supernatant was then removed and the wells were washed 4 times with 120 μL wash buffer (1×DPBS, 0.2% BSA, and 0.05% Tween-20). Pool RNA was heated to 90° C. for 3 minutes and cooled to room temperature for 10 minutes to refold. In round 1, a positive selection step was conducted. Briefly, 1×10¹⁴ molecules (0.2 nmoles) of pool RNA were incubated in 100 μL binding buffer (1×DPBS and 0.05% Tween-20) in the wells with immobilized protein target for 1 hour. The supernatant was then removed and the wells were washed 4× with 120 μL wash buffer. In subsequent rounds a negative selection step was included. The pool RNA was also incubated for 30 minutes at room temperature in empty wells to remove any plastic binding sequences from the pool before the positive selection step. The number of washes was increased after round 4 to increase stringency. In all cases, the pool RNA bound to immobilized h-IgE was reverse transcribed directly in the selection plate after by the addition of RT mix (3′ primer, STC.104.102.A, and Thermoscript RT, Invitrogen) followed by incubated at 65° C. for 1 hour. The resulting cDNA was used as a template for PCR (Taq polymerase, New England Biolabs) “Hot start” PCR conditions coupled with a 60° C. annealing temperature were used to minimize primer-dimer formation. Amplified pool template DNA was desalted with a Centrisep column according to the manufacturer's recommended conditions and used to program transcription of the pool RNA for the next round of selection. The transcribed pool was gel purified on a 10% polyacrylamide gel every round.

rRmY pool selection against h-IgE was enriched after 4 rounds over the naive pool. The selection stringency was increased and the selection was continued for 2 more rounds. At round 6 the pool K_{D} is approximately 500 nM or higher. The pools were cloned using TOPO TA cloning kit (Invitrogen) and submitted for sequencing. The pool contained one dominant clone (AMX(123).A1)—which made up 71% of the clones sequenced. Three additional clones were tested and showed a higher extent of binding than the dominant clone. The K_{D}s for the pools were calculated to be approximately 500 nM. The dissociations constants were also calculated as described above. Table 18 shows the rRmY pool clones after Round 6 of selection to h-IgE where the dominant clone was AMX(123).A1 making up 40% of the 96 clones, along with 8 other sequence families.

TABLE 18
Corresponding cDNAs of the Individual Clone Sequence of rRmY Pool
Clones After Round 6 of Selection to h-IgE.

SEQ ID No.355 ARX(123).A1
GGGAGAGGAGAGAACGTTCTACGATCTGGGCGAGCCAGTCTGACTGAGGAAGCGCTGTCGATCGATCGATCGATGAAGGGCG
SEQ ID No.356 ARX34P1.B07
GGGAGAGGAGAGAACGTTCTACGAAGAAGATATGAGAGAAAGGATTAAGAGACGCTGTCGATCGATCGATCGATGAAGGGCG
SEQ ID No.357 ARX34P1.A07
GGGAGAGGAGAGAACGTTCTACGAAAAAGATATGAGAGAAAGGATTAAGAGACGCTGTCGATCGATCGATCGATGAAGGGCG
SEQ ID No.358 ARX34P1.A01
GGGAGAGGAGAGAACGTTCTACGAAAAAGATATGAGAGAAAGGATTAAGAGGCGCTGTCGATCGATCGATCGATGAAGGGCG
SEQ ID No.359 ARX34P1.G05
GGGAGAGGAGAGAACGTTCTACGAAAAAGACATGAGAGAAAGGATTAAGAGACGCTGTCGATCGATCGATCGATGAAGGGCG
SEQ ID No.360 ARX34P1.F09
GGGAGAGGAGAGAACGTTCTACNAAAAAGTATATGAGAGAAAGGATTAANAGACGCTGTCGATCGATCGATCGATGAAGGGCG
SEQ ID No.361 ARX34P1.E02
GGGAGAGGAGAGAACGTTCTACGAAAAAGATATGAGAGAAAAGGATTGAGAGATGCTGTCGATCGATCGATCGATGAAGGGCG
SEQ ID No.362 ARX34P1.G02
GGGAGAGGAGAGCACGTTCTACGAAAAAGATATGGAGAGAAAGGATTAAGAGACGCTGTCGATCGATCGATCGATGAAGGGCG
SEQ ID No.363 ARX34P1.A04
GGGAGAGGAGAGAACGTTCTACGAAAAAGATATGAGAGAAAGGATTAAAAGAGACGCTGTCATCGATCGATCGATGAAGGGCG
SEQ ID No.364 ARX34P1.G06
GGGAGAGGAGAGAACGTTCTACGAANAAGATACATAGTAGAAAGGATTAATAAGACGCTGTCGATCGATCGATCGATGAAGGGCG
SEQ ID No.365 ARX34P1.E05
GGGAGAGGAGAGAACGTTCTACAGGCGTGTTGGTAGGGTACGACGAGGCATGCGCTGTCGATCGATCGATCGATGAAGGGCG
SEQ ID No.366 ARX34P1.B11
GGGAGAGGAGAGAACGTTCTACGCAAAAATGTGATGCGAGGTAATGGAACGCCGCTGTCGATCGATCGATCGATTGAAGGGCG
SEQ ID No.367 ARX34P1.B01
GGGAGAGGAGAGAACGTTCTACGGACCTCAGCGATAGGGGTTGAAACCGACACGCTGTCGATCGATCGATCGATGAAGGGCG
SEQ ID No.368 ARX34P1.E06
GGGAGAGGAGAGAACGTTCTACATGGTCGGATGCTGGGGAGTAGGCAAGGTTCGCTGTCGATCGATCGATCGATGAAGGGCG
SEQ ID No.369 ARX34P1.C12
GGGAGAGGAGAGAACGTTCTACGTATCGGCGAGCGAAGCATCCGGGAGCGTTCGCTGTCGATCGATCGATCGATGAAGGGCG
SEQ ID No.370 ARX34P1.C09
GGGAGAGGAGAGAACGTTCTACGTATTGGCGCGCGAAGCATCCGGGAGCGTTCGCTGTCGATCGATCGATCGATGAAGGGCG
SEQ ID No.371 ARX34P1.A11
GGGAGAGGAGAGAACGTTCTACTTATACCTGACGGCCGGAGGCGCATAGGTGCGCTGTCGATCGATCGATCGATGAAGGGCG
SEQ ID No.372 ARX34P1.H09
GGGAGAGGAGAGAACGTTCTACATGGTCGGATGCTGGGGAGTAGGCAAGGTTCGCTGTCGATCGATCGATCGATGAAGGGCG
SEQ ID No.373 ARX34P1.805
GGGAGAGGAGAGAACGTTCTACACGAGAGTACTGAGGCGCTTGGTACAGAGTCGCTGTCGATCGATCGATCGATGAAGGGCG
SEQ ID No.374 ARX34P1.B10
GGGAGAGGAGAGAACGTTCTACAGAAGGTAGAAAAAGGATAGCTGTGAGAAGCGCTGTCGATCGATCGATCGATGAAGGGCG
SEQ ID No.375 ARX34P1.CO1
GGGAGAGGAGAGAACGTTCTACTGAGGGATAATACGGGTGGGATTGTCTTCCCGCTGTCGATCGATCGATCGATGAAGGGCG
SEQ ID No.376 ARX34P1.D04
GGGAGAGGAGAGAACGTTCTACATTGAGCGTTGAAGTTGGGGAAGCTCCGAGGCCGCTGTCGATCGATCGATCGATGAAGGGCG
SEQ ID No.377 ARX34P1.E02
GGGAGAGGAGAGAACGTTCTACGCGGAGATATACAGCGAGGTAATGGAACGCCGCTGTCGATCGATCGATCGATGAAGGGCG
SEQ ID No.378 ARX34P1.F01
GGGAGAGGAGAGAACGTTCTACGAAGACAGCCCAATAGCGGCACGGAACTTGCGCTGTCGATCGATCGATCGATGAAGGGCG
SEQ ID No.379 ARX34P1.G03
GGGAGAGGAGAGAACGTTCTACCGGTTGAGGGCTCGCGTGGAAGGGCCAACACGCGCTGTCGATCGATCGATCGATGAAGGGCG
SEQ ID No.380 ARX34P1.E01
GGGAGAGGAGAGAACGTTCTACATATCAATAGACTCTTGACGTTTGGGTTTGCGCTGTCGATCGATCGATCGATGAAGGGCG
SEQ ID No.381 ARX34P1.H02
GGGAGAGGAGAGAACGTTCTACAGTGAAGGAAAAGTAAGTGAAGGTGTGCGCTGTCGATCGATCGATCGATGAAGGGCG
SEQ ID No.382 ARX34P1.H03
GGGAGAGGAGAGAACGTTCTACGGATGAAATGAGTGTCTGCGATAGGTTAAGCGCTGTCGATCGATCGATCGATGAAGGGCG
SEQ ID No.383 ARX34P1.N10
GGGAGAGGAGAGAACGTTCTACGGAAGGAAATGTGTGTCTGCGATAGGTTAAGCGCTGTCGATCGATCGATCGATGAAGGGCG

### Example 9

### rRmY and rGmH 2′-OMe SELEX™ Against PDGF-BB

Selections were performed to identify aptamers containing 2′-OMe C, U and 2′-OH G, A (rRmY), and the other 2′-O-Methyl A, C, and U and 2′-OH G (rGmH). All selections were direct selections against human PDGF-BB protein target which had been immobilized on a hydrophobic plate. Selections yielded pools significantly enriched for h-_PDGF-BB binding versus naive, unselected pool. Individual clone sequences for PDGF-BB are reported herein.

Pool Preparation. A DNA template with the sequence 5′-GGGAGAGGAGAGAACGTTCTACNNNNNNNNNNNNNNNNNNNNNNNNNNNNC GCTGTCGATCGATCGATCGATG-3′ (SEQ ID NO:459) was synthesized using an ABI EXPEDITE™ DNA synthesizer, and deprotected by standard methods. The templates were amplified with the primers PB.118.95.G 5′-GGGAGAGGAGAGAACGTTCTAC-3′ (SEQ ID NO:460) and STC.104.102.A 5′-CATCGATCGATCGATCGACAGC-3′(SEQ ID NO:461) and then used as a template (200 nm template was used for round 1, and for subsequent rounds approximately 50 nM, a 1/10 dilution of an optimized PCR reaction, using conditions described herein, was used) for in vitro transcription with Y639F single mutant T7 RNA polymerase. Transcriptions were done using 200 mM HEPES, 40 mM DTT, 2 mM spermidine, 0.01% TritonX-100, 10% PEG-8000, 5 mM MgCl₂, 1.5 mM MnCl₂, 500 μM NTPs, 500 μM GMP, 0.01 units/μl inorganic pyrophosphatase, and Y639F single mutant T7 polymerase. Two different compositions were transcribed rRmY and rGmH. Selection. Each round of selection was initiated by immobilizing 20 pmoles of PDGF-BB to the surface Nunc Maxisorp hydrophobic plates for 2 hours at room temperature in 100 μL of 1×Dulbecco's PBS. The supernatant was then removed and the wells were washed 4 times with 120 μL wash buffer (1×DPBS, 0.2% BSA, and 0.05% Tween-20). Pool RNA was heated to 90° C. for 3 minutes and cooled to room temperature for 10 minutes to refold. In round 1, a positive selection step was conducted. Briefly, 1×10¹⁴ molecules (0.2 nmoles) of pool RNA were incubated in 100 μL binding buffer (1×DPBS and 0.05% Tween-20) in the wells with immobilized protein target for 1 hour. The supernatant was then removed and the wells were washed 4× with 120 μL wash buffer. In subsequent rounds a negative selection step was included. The pool RNA was also incubated for 30 minutes at room temperature in empty wells to remove any plastic binding sequences from the pool before the positive selection step. The number of washes was increased after round 4 to increase stringency. In all cases, the pool RNA bound to immobilized PDGF-BB was reverse transcribed directly in the selection plate after by the addition of RT mix (3′ primer, STC.104.102.A, and Thermoscript RT, Invitrogen) followed by incubated at 65° C. for 1 hour. The resulting cDNA was used as a template for PCR (Taq polymerase, New England Biolabs) “Hot start” PCR conditions coupled with a 60° C. annealing temperature were used to minimize primer-dimer formation. Amplified pool template DNA was desalted with a Centrisep column according to the manufacturer's recommended conditions and used to program transcription of the pool RNA for the next round of selection. The transcribed pool was gel purified on a 10% polyacrylamide gel every round.

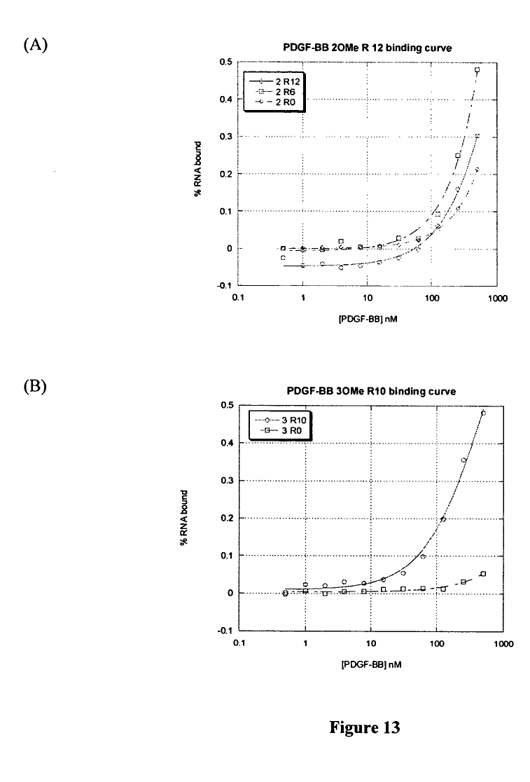

Although the naïve pool does bind to PDGF-BB, the rRmY PDGF-BB selection was enriched after 4 rounds over the naive pool. The selection stringency was increased and the selection was continued for 8 more rounds. At round 12 the pool is enriched over the naïve pool, but the K_{D} is very high. The rGmH selection was enriched over the naive pool binding at round 10. The pool K_{D} is also approximately 950 nM or higher. The pools were cloned using TOPO TA cloning kit (Invitrogen) and submitted for sequencing. After 12 rounds of PDGF-BB pool selection clones were transcribed and sequenced. Table 19 shows the clone sequences. FIG. 13(A) shows a binding plot of round 12 pools for rRmY pool PDGF-BB selection and FIG. 13(B) shows a binding plot of round 10 pools for rGmH pool PDGF-BB selection. Dissociation constants were again measured using the sandwich filter binding technique. Dissociation constants (K_{D}s) were estimated fitting the data to the equation: fraction RNA bound=amplitude*K_{D}/(K_{D}+[PDGF-BB]).

TABLE 19

Corresponding cDNAs of the Individual Clone
Sequence of rRmY Pool Clones After Round 12 of
Selection to PDGF-BB.

SEQ ID No.384 PDGF-BB ARX36.SCK.E05
GGGAGAGGAGAGAACGTTCTACATCCTTGCGTATGATCGGCATCGTAAGA
CACGCTGTCGATCGATCGATCGATGAAGGGCG
SEQ ID No.385 PDGF-BB ARX36.SCK.F05
GGGAGAGGAGAGAACGTTCTACATCCTTGCGTATGATCGGCATCGTAAGA
CACGCTGTCGATCGATCGATCGATGAAGGGCG
SEQ ID No.386 PDGF-BB ARX36.SCK.E01
GGGAGAGGAGAGAACGTTCTACGATCGAAGTCGTGACAGAAACCACTCGC
TGTCGATCGATCGATCGATGAAGGGCG
SEQ ID No.387 PDGF-BB ARX36.SCK.F01
GGGAGAGGAGAGAACGTTCTACGATCGAAGTCGTGACAGAAACCACTCGC
TGTCGATCGATCGATCGATGAAGGGCG
SEQ ID No.388 PDGF-BB ARX36.SCK.G01
GGGAGAGGAGAGAACGTTCTACGGAAAAGGTTGGCGAAACGAAGAAGAAT
TTCGCTGTCGATCGATCGATCGATGAAGGGCG
SEQ ID No.389 PDGF-BB ARX36.SCK.G02
GGGAGAGGAGAGAACGTTCTACGGAAAAGGTTGGCGAAACGAAGAANAAT
TTCGCTGTCGATCGATCGATCGATGAAGGGCG
SEQ ID No.390 PDGF-BB ARX36.SCK.F04
GGGAGAGGAGAGAACGTTCTACTGGGAGTTGCGGTGTTTTGCGGTGGATT
TGACGCTGTCGATCGATCGATCGATGAAGGGCG
SEQ ID No.391 PDGF-BB ARX36.SCK.E04
GGGAGAGGAGAGAACGTTCTACTGGGAGTTGCGGTGTTTTGCGGTGGATT
TGACGCTGTCGATCGATCGATCGATGAAGGGCG
SEQ ID No.392 PDGF-BB ARX36.SCK.F02
GGGAGAGGAGAGAACGCTCTACAAGATTGTAGATCAACAGCGAAGGCGTG
GGCGCTGTCGATCGATCGATCGATGAAGGGCG
SEQ ID No.393 PDGF-BB ARX36.SCK.E02
GGGAGAGGAGAGAACGCTCTACAAGATTGTAGATCAACAGCGAAGGCGTG
GGCGCTGTCGATCGATCGATCGATGAAGGGCG
SEQ ID No.394 PDGF-BB ARX36.SCK.A02
GGGAGAGGAGAGAACGTTCTACAAANAAGATNNCCANCNNGAGANAAAGG
AGCGCTGTCGATCGATCGATCGATGAAGGGCG
SEQ ID No.395 PDGF-BB ARX36.SCK.A03
GGGAGAGGAGAGAACGTTCTACAAACATCGAAGATCGAACTGAAAAGGAG
GGCGCTGTCGATCGATCGATCGATGAAGGGCG
SEQ ID No.396 PDGF-BB ARX36.SCK.A06
GGGAGAGGAGAGAACGTTCTACATGTGCATGCAAGGTGGGGCTGACACGA
GCCGCTGTCGATCGATCGATCGATGAAGGGCG
SEQ ID No.397 PDGF-BB ARX36.SCK.B01
GGGAGAGGAGAGAACGTTCTACAAGGAGTAGATCGACAGAATAGAAAAAT
CGCTGTCGATCGATCGATCGATGAAGGGCG
SEQ ID No.398 PDGF-BB ARX36.SCK.B02
GGGAGAGGAGAGAACGTTCTACAAAAGGTAAGGTCAAAAAAGCGCAACGT
TGACGCTGTCGATCGATCGATCGATGAAGGGCG
SEQ ID No.399 PDGF-BB ARX36.SCK.D04
GGGAGAGGAGAGAACGTTCTACAAAAGGAGGCGAAATAAGTGAGACAATG
TGCGCTGTCGATCGATCGATCGATGAAGGGCG
SEQ ID No.400 PDGF-BB ARX36.SCK.B04
GGGAGAGGAGAGAACGTTCTACAAAAATCCACAAACATAGCTGTAATTGC
TCGCTGTCGATCGATCGATCGATGAAGGGCG
SEQ ID No.401 PDGF-BB ARX36.SCK.B05
GGGAGAGGAGAGACGTTCTACAAGAACATATAACATTTTGGTTGAGAGCA
ACGCTGTCGATCGATCGATCGATGAAGGGCG
SEQ ID No.402 PDGF-BB ARX36.SCK.D03
GGGAGAGAGAGAACGTTCTACAAGAGTCNACGATTTCNATCACAAATGTG
GCTGCTGTCNATCGATCGATCNATGAAGGGCG
SEQ ID No.403 PDGF-BB ARX36.SCK.C01
GGGAGAGGAGAGAACGTTCTACAAGCAAGCAAAAAAAGTATCGACAGAAG
TGGCGCTGTCGATCGATCGATCGATGAAGGGCG
SEQ ID No.404 PDGF-BB ARX36.SCK.D06
GGGAGAGGAGAGAACGTTCTACAAGTAATATCAGAGCAATCGGAATAAGA
GTCGCTGTCGATCGATCGATCGATGAAGGGCG
SEQ ID No.405 PDGF-BB ARX36.SCK.D02
GGGAGAGGAGAGAACGTTCTACAGACTTCGATGCGATGGATTTGGAAATG
TGCGCTGTCGATCGATCGATCGATGAAGGGCG
SEQ ID No.406 PDGF-BB ARX36.SCK.C03
GGGAGAGGAGAGAACGTTCTACAGAAAGAATTACAGGAACAAATACACGT
GCGGCTGTCGATCGATCGATCGATGAAGGGCG
SEQ ID No.407 PDGF-BB ARX36.SCK.F06
GGGAGAGGAGAGAACGTTCTACAGAAATCAATCGAGGTGATCGTTATATA
GGCGCTGTCGATCGATCGATCGATGAAGGGCG
SEQ ID No.408 PDGF-BB ARX36.SCK.C04
GGGAGAGGAGAGAACGTTCTACAGATTTGGATCGACAATCTCGTAGAAGA
GACGCTGTCGATCGATCGATCGATGAAGGGCG
SEQ ID No.409 PDGF-BB ARX36.SCK.C06
GGGAGAGGAGAGAACGTTCTACAATGCAAGTTTAAGTGTGGTGTCAAACG
CACGCTGTCGATCGATCGATCGATGAAGGGCG
SEQ ID No.410 PDGF-BB ARX36.SCK.G03
GGGAGAGGAGAGAACGTTCTACAAATAAAGACACGAAGATCGACGGAGAC
TCGCTGTCGATCGATCGATCGATGAAGGGCG
SEQ ID No.411 PDGF-BB ARX36.SCK.F03
GGGAGAGGAGAGAACGTTCTACGAAGATGTGTTTAAGAATCGAGGTTTTC
GACGCTGTCGATCGATCGATCGATGAAGGGCG
SEQ ID No.412 PDGF-BB ARX36.SCK.C02
GGGAGAGGAGAGAACGTTCTACGAGTTGGCACGCATGTATAGGTATTTTG
GCGCTGTCGATCGATCGATCGATGAAGGGCG
SEQ ID No.413 PDGF-BB ARX36.SCK.B03
GGGAGAGGAGAGAACGTTCTACGAAAAAAAGAGATGAGAGAAAGGATTAA
GAGACGCTGTCGATCGATCGATCGATGAAGGGCG
SEQ ID No.414 PDGF-BB ARX36.SCK.B06
GGGAGAGGAGAGAACGTTCTACGAAAAGGAAAAAAAACGATCGGCAGAGT
CCCGCTGTCGATCGATCGATCGATGAAGGGCG
SEQ ID No.415 PDGF-BB ARX36.SCK.C05
GGGAGAGGAGAGAACGTTCTACGATTAAGGAAACATTTACGCGAATACAT
GACGCTGTCGATCGATCGATCGATGAAGGGCG
SEQ ID No.416 PDGF-BB ARX36.SCK.D01
GGGAGAGGAGAGAACGTTCTACGACGTTTGCTCTGAAAATAGGACAGAAG
GCGCTGTCGATCGATCGATCGATGAAGGGCG
SEQ ID No.417 PDGF-BB ARX36.SCK.E03
GGGAGAGGAGAGAACGTTCTACGAAGATGTGTTTAAGAATCGAGGTTTTC
GACGCTGTCGATCGATCGATCGATGAAGGGCG
SEQ ID No.418 PDGF-BB ARX36.SCK.A04
GGGAGAGGAGAGAACGTTCTACCGAGATCGAAAGGTAAGAGAAAATTCAT
GGCGCTGTCGATCGATCGATCGATGAAGGGCG
SEQ ID No.419 PDGF-BB ARX36.SCK.A05
GGGAGAGGAGAGAACGTTCTACTAAGATTCGTCGTTCAGACAGAGAAAGC
GACGCTGTCGATCGATCGATCGATGAAGGGCG

TABLE 20

Corresponding cDNAs of the Individual Clone
Sequence of rGmH Pool Clones After Round 10
of Selection to PDGF-BB.

SEQ ID No.420 PDGF-BB ARX36.SCK.E08.M13F
GGGAGAGGAGAGAACGTTCTACCTTGGCGACGATCTGTGACCTGAATTTT
TGTCCGCTGTCGATCGATCGATCGATGAAGGGCG
SEQ ID No.421 PDGF-BB ARX36.SCK.F08.M13F
GGGAGAGGAGAGAACGTTCTACCTTGGCGACGATCTGTGACCTGAATTTT
TGTCCGCTGTCGATCGATCGATCGATGAAGGGCG
SEQ ID No.422 PDGF-BB ARX36.SCK.E09.M13F
GGGAGAGGAGAGAACGTTCTACCTTGGTCTCAGCAGCTTTTAACAAAGTA
TCCCGCTGTCGATCGATCGATCGATGAAGGGCG
SEQ ID No.423 PDGF-BB ARX36.SCK.F09.M13F
GGGAGAGGAGAGAACGTTCTACCTTGGTCTCAGCAGCTTTTAACAAAGTA
TCCCGCTGTCGATCGATCGATCGATGAAGGGCG
SEQ ID No.424 PDGF-BB ARX36.SCK.F07.M13F
GGGAGAGGAGAGAACGTTCTACCGCTATTTTGTTCATTGAAGGACTTGTC
ACGCTGTCGATCGATCGATCGATGAAGGGCG
SEQ ID No.425 PDGF-BB ARX36.SCK.E07.M13F
GGGAGAGGAGAGAACGTTCTACCGCTATTTTGTTCATTGAAGGACTTGTC
ACGCTGTCGATCGATCGATCGATGAAGGGCG
SEQ ID No.426 PDGF-BB ARX36.SCK.E11.M13F
GGGAGAGGAGAGAACGTTCTACCCTATTGAGGTTGATTGGAAGTGCCTAT
GTCGCTGTCGATCGATCGATCGATGAAGGGCG
SEQ ID No.427 PDGF-BB ARX36.SCK.F11.M13F
GGGAGAGGAGAGAACGTTCTACCCTATTGAGGTTGATTGGAAGTGCCTAT
GTCGCTGTCGATCGATCGATCGATGAAGGGCG
SEQ ID No.428 PDGF-BB ARX36.SCK.F10.M13F
GGGAGAGGAGAGAACGTTCTACTGAAGATGTTATGATGATTGACGAGGAG
GCGCTGTCGATCGATCGATCGATGAAGGGCG
SEQ ID No.429 PDGF-BB ARX36.SCK.E10.M13F
GGGAGAGGAGAGAACGTTCTACTGAAGATGTTATGATGATTGACGAGGAG
GCGCTGTCGATCGATCGATCGATGAAGGGCG
SEQ ID No.430 PDGF-BB ARX36.SCK.E12.M13F
GGGAGAGGAGAGAACGTTCTACTGTCTGAGTGTCGCCGCCTTGTGTGATG
TTCGCTGTCGATCGATCGATCGATGAAGGGCG
SEQ ID No.431 PDGF-BB ARX36.SCK.F12.M13F
GGGAGAGGAGAGAACGTTCTACTGTCTGAGTGTCGCCGCCTTGTGTGATG
TTCGCTGTCGATCGATCGATCGATGAAGGGCG
SEQ ID No.432 PDGF-BB ARX36.SCK.A07.M13F
GGGAGAGGAGAGAACGTTCTACGTGATGGCTGTGAATGAGGTAGTTCGAA
TACGCTGTCGATCGATCGATCGATGAAGGGCG
SEQ ID No.433 PDGF-BB ARX36.SCK.C12.M13F
GGGAGAGGAGAGAACGTTCTACGTGAAATCAAGGTTGTTAATTTGGGGAA
TCGCTGTCGATCGATCGATCGATGAAGGGCG
SEQ ID No.434 PDGF-BB ARX36.SCK.B07.M13F
GGGAGAGGAGAGAACGTTCTACGTATAAGGCCGTAACCGGGTAGCGAGTG
GTCGCTGTCGATCGATCGATCGATGAAGGGCG
SEQ ID No.435 PDGF-BB ARX36.SCK.A09.M13F
GGGAGAGGAGAGAACGTTNTACGTGGGCGAAGGAGCTGCGGGCGTTGNAG
TTTGCTGTCGATCGATCGATCGATGAAGGGCG
SEQ ID No.436 PDGF-BB ARX36.SCK.A11.M13F
GGGAGAGGAGAGAACGTTCTACGTCATCCTAGTCTGAGATCGGATTTTCT
TGCGCTGTCGATCGATCGATCGATGAAGGGCG
SEQ ID No.437 PDGF-BB ARX36.SCK.C09.M13F
GGGAGAGGAGAGAACGTTCTACGTTTGCGAGTGTGGTCGACGCTGAATGC
GGCGCTGTCGATCGATCGATCGATGAAGGGCG
SEQ ID No.438 PDGF-BB ARX36.SCK.A08.M13F
GGGAGAGGAGAGAACGTTCTACGGATTGATAGGGATTGAGATGAGGTCTT
GTCGCTGTCGATCGATCGATCGATGAAGGGCG
SEQ ID No.439 PDGF-BB ARX36.SCK.D07.M13F
GGGAGAGGAGAGAACGTTCTACGATGTCGTGTTAGATTACTTATTGCTAT
CTGCTGTCGATCGATCGATCGATGAAGGGCG
SEQ ID No.440 PDGF-BB ARX36.SCK.D08.M13F
GGGAGAGGAGAGAACGTTCTACGATGCCTGGCGGAAACGGAGCCTGGGAT
TTCGCTGTCNATCGATCGATCGATGAAGGGCG
SEQ ID No.441 PDGF-BB ARX36.SCK.B11.M13F
GGGAGAGGAGAGAACGTTCTACGAGGATTTGACGTGTGTGTGCTAGAGTA
CGCTGTCGATCGATCGATCGATGAAGGGCG
SEQ ID No.442 PDGF-BB ARX36.SCK.D09.M13F
GGGAGAGGAGAGAACGTTCTACGAGTATTATGCGTCCCTTGAGGATACAC
GGCGCTGTCGATCGATCGATCGATGAAGGGCG
SEQ ID No.443 PDGF-BB ARX36.SCK.B10.M13F
GGGAGAGGAGAGAACGTTCTACAGGGATAACTGTAGCGATGAAAGTAAAC
GATGCTGTCGATCGATCGATCGATGAAGGGCG
SEQ ID No.444 PDGF-BB ARX36.SCK.C10.M13F
GGGAGAGGAGAGAACGTTCTACAAGAAGTGTGGCCGCAGAGACGAAATGC
ACGCTGTCGATCGATCGATCGATGAAGGGCG
SEQ ID No.445 PDGF-BB ARX36.SCK.A10.M13F
GGGAGAGGAGAGAACGTTCTACCCATATCTTCCTTCTTTATTCCGTTAGT
TGCCGCTGTCGATCGATCGATCGATGAAGGGCG
SEQ ID No.446 PDGF-BB ARX36.SCK.B09.M13F
GGGAGAGGAGAGAACGTTCTACCTGTGTTGATGCTTCCGTTTGAGATTGC
CCCGCTGTCGATCGATCGATCGATGAAGGGCG
SEQ ID No.447 PDGF-BB ARX36.SCK.B12.M13F
GGGAGAGGAGAGAACGTTCTACCNGTAAGANAANCTATTTTAGCCCTTGN
NCTGCGCTGTCGATCGATCGATCGATGAAGGGCG
SEQ ID No.448 PDGF-BB ARX36.SCK.C08.M13F
GGGAGAGGAGAGAACGTTCTACCCTTGTCCTCCAATCCTCTTTTGACTCT
TGCCGCTGTCGATCGATCGATCGATGAAGGGCG
SEQ ID No.449 PDGF-BB ARX36.SCK.D12.M13F
GGGAGAGGAGAGAACGTTCTACCTGATTTTGTCACTGGATTCCGATGGCT
TTCGCTGTCGATCGATCGATCGATGAAGGGCG
SEQ ID No.450 PDGF-BB ARX36.SCK.C11.M13F
GGGAGAGGAGAGAACGTTCTACTGTAATAAGGGATGCGTCAGGAACCTGT
GTTCGCTGTCGATCGATCGATCGATGAAGGGCG
SEQ ID No.451 PDGF-BB ARX36.SCK.D11.M13F
GGGAGAGGAGAGAACGTTCTACTGCTTTCCGGGAATTTGTTTGTTTGCTT
CCGCTGTCGATCGATCGATCGATGAAGGGCG
SEQ ID No.452 PDGF-BB ARX36.SCK.C07.M13F
GGGAGAGGAGAGAACGTTCTACTTCGTCGGTTCACTTTTCTTCGTGTAGT
GTCGCTGTCGATCGATTGATCGATGAAGGGCG
SEQ ID No.189 PDGF-BB ARX36.SCK.A12.M13F
GGGAGAGGAGAGAACGTTCTACTATGAAGGGTTTTAAAGATGACACATTA
GCCGCTGTCGATCGATCGATCGATGAAGGGCG

The present invention having been described by detailed description and the foregoing non-limiting examples, is now defined by the spirit and scope of the following claims.

What is claimed is:

1. A method for identifying nucleic acid ligands comprising a modified nucleotide to a target molecule comprising:
a) preparing a transcription reaction mixture comprising a mutated polymerase, one or more 2′-modified nucleotide triphosphates (NTPs), magnesium ions and one or more oligonucleotide transcription templates;
b) preparing a candidate mixture of single-stranded nucleic acids by transcribing the one or more oligonucleotide transcription templates under conditions whereby the mutated polymerase incorporates at least one of the one or more modified nucleotides into each nucleic acid of said candidate mixture, wherein each nucleic acid of said candidate mixture comprises a 2′-modified nucleotide selected from the group consisting of a 2′-position modified pyrimidine and a 2′-position modified purine;
c) contacting the candidate mixture with said target molecule;
d) partitioning the nucleic acids having an increased affinity to the target molecule relative to the candidate mixture from the remainder of the candidate mixture; and
e) amplifying the increased affinity nucleic acids, in vitro, to yield a ligand-enriched mixture of nucleic acids, whereby nucleic acid ligands of the target molecule are identified.

2. The method of claim 1, wherein the one or more 2′-modified nucleotides are selected from the group consisting of 2′-OH, 2′-deoxy, 2′-O-methyl, 2′-NH₂, 2′-F, and 2′-methoxy ethyl modifications.

3. The method of claim 1, wherein the one or more 2′-modified nucleotides are a 2′-β-methyl modification.

4. The method of claim 1, wherein the one or more 2′-modified nucleotides are a 2′-F modification.

5. The method of claim 1, wherein the mutated polymerase is a mutated T7 RNA polymerase.

6. The method of claim 5, wherein the mutated T7 RNA polymerase comprises a mutation at position 639 from a tyrosine residue to a phenylalanine residue (Y639F).

7. The method of claim 5, wherein the mutated T7 RNA polymerase comprises a mutation at position 784 from a histidine residue to an alanine residue (H784A).

8. The method of claim 5, wherein the mutated T7 RNA polymerase comprises a mutation at position 639 from a tyrosine residue to a phenylalanine residue and a mutation at position 784 from a histidine residue to an alanine residue (Y639F/H784A).

9. The method of claim 1, wherein the oligonucleotide transcription template further comprises a leader sequence incorporated into a fixed region at the 5′ end of the oligonucleotide transcription template.

10. The method of claim 9, wherein the leader sequence comprises an all-purine leader sequence.

11. The method of claim 10, wherein the all-purine leader sequence has a length selected from the group consisting of at least 6 nucleotides long; at least 8 nucleotides long; at least 10 nucleotides long; at least 12 nucleotides long; and at least 14 nucleotides long.

12. The method of claim 1, wherein the transcription reaction mixture further comprises manganese ions.

13. The method of claim 12, wherein the concentration of magnesium ions is between 3.0 and 3.5 times greater than the concentration of manganese ions.

14. The method of claim 1, wherein each NTP is present at a concentration of 0.5 mM, the concentration of magnesium ions is 5.0 mM, and the concentration of manganese ions is 1.5 mM.

15. The method of claim 1, wherein each NTP is present at a concentration of 1.0 mM, the concentration of magnesium ions is 6.5 mM, and the concentration of manganese ions is 2.0 mM.

16. The method of claim 1, wherein each NTP is present at a concentration of 2.0 mM, the concentration of magnesium ions is 9.6 mM, and the concentration of manganese ions is 2.9 mM.

17. The method of claim 1, wherein the transcription reaction mixture further comprises 2′-OH GTP.

18. The method of claim 1, wherein the transcription reaction mixture further comprises a polyalkylene glycol.

19. The method of claim 18, wherein the polyalkylene glycol is polyethylene glycol (PEG).

20. The method of claim 1, wherein the transcription reaction mixture further comprises GMP.

21. The method of claim 1 further comprising
f) repeating steps d) and e).

22. A nucleic acid ligand to thrombin identified according to the method of claim 1.

23. A nucleic acid ligand to vascular endothelial growth factor (VEGF) identified according to the method of claim 1.

24. A nucleic acid ligand to IgE identified according to the method of claim 1.

25. A nucleic acid ligand to IL-23 identified according to the method of claim 1.

26. A nucleic acid ligand to platelet-derived growth factor-BB (PDGF-BB) identified according to the method of claim 1.

27. The method of claim 1, wherein the 2′ modified nucleotide triphosphates comprise a mixture of 2′-OH adenosine triphosphate (ATP), 2′-OH guanosine triphosphate (GTP), 2′-O-methyl cytidine triphosphate (CTP) and 2′-O-methyl uridine triphosphate (UTP).

28. The method of claim 1, wherein the 2′ modified nucleotide triphosphates comprise a mixture of 2′-deoxy purine nucleotide triphosphates and 2′-O-methylpyrimidine nucleotide triphosphates.

29. The method of claim 1, wherein the 2′ modified nucleotide triphosphates comprise a mixture of 2′-O-methyl adenosine triphosphate (ATP), 2′-OH guanosine triphosphate (GTP), 2′-O-methyl cytidine triphosphate (CTP) and 2′-O-methyl uridine triphosphate (UTP).

30. The method of claim 1, wherein the 2′ modified nucleotide triphosphates comprise a mixture of 2′-O-methyl adenosine triphosphate (ATP), 2′-O-methyl cytidine triphosphate (CTP) and 2′-O-methyl uridine triphosphate (UTP), 2′-O-methyl guanosine triphosphate (GTP) and deoxy guanosine triphosphate (GTP), wherein the deoxy guanosine triphosphate comprises a maximum of 10% of the total guanosine triphosphate population.

31. The method of claim 1, wherein the 2′ modified nucleotide triphosphates comprise a mixture of 2′-O-methyl adenosine triphosphate (ATP), 2′-F guanosine triphosphate (GTP), 2′-O-methyl cytidine triphosphate (CTP) and 2′-O-methyl uridine triphosphate (UTP).

32. The method of claim 1, wherein the 2′ modified nucleotide triphosphates comprise a mixture of 2′-deoxy adenosine triphosphate (ATP), 2′-O-methyl guanosine triphosphate (GTP), 2′-O-methyl cytidine triphosphate (CTP) and 2′-O-methyl uridine triphosphate (UTP).

33. A method of preparing a nucleic acid comprising one or more modified nucleotides comprising:
(a) preparing a transcription reaction mixture comprising a mutated polymerase, one or more 2′-modified nucleotide triphosphates (NTPs), magnesium ions and one or more oligonucleotide transcription templates; and
(b) contacting the one or more oligonucleotide transcription templates with the mutated polymerase under conditions whereby the mutated polymerase incorporates the one or more 2′-modified nucleotides into a nucleic acid transcription product.

34. The method of claim 33, wherein the one or more 2′-modified nucleotides are selected from the group consisting of 2′-OH, 2′-deoxy, 2′-O-methyl, 2′-NH₂, 2′-F, and 2′-methoxy ethyl modifications.

35. The method of claim 33, wherein the one or more 2′-modified nucleotides are a 2′-O-methyl modification.

36. The method of claim 33, wherein the one or more 2′-modified nucleotides are a 2′-F modification.

37. The method of claim 33, wherein the mutated polymerase is a mutated T7 RNA polymerase.

38. The method of claim 37, wherein the mutated T7 RNA polymerase comprises a mutation at position 639 from a tyrosine residue to a phenylalanine residue (Y639F).

39. The method of claim 37, wherein the mutated T7 RNA polymerase comprises a mutation at position 784 from a histidine residue to an alanine residue (H784A).

40. The method of claim 37, wherein the mutated T7 RNA polymerase comprises a mutation at position 639 from a tyrosine residue to a phenylalanine residue and a mutation at position 784 from a histidine residue to an alanine residue (Y639F/H784A).

41. The method of claim 33, wherein the oligonucleotide transcription template further comprises a leader sequence incorporated into a fixed region at the 5′ end of the oligonucleotide transcription template.

42. The method of claim 41, wherein the leader sequence comprises an all-purine leader sequence.

43. The method of claim 42, wherein the all-purine leader sequence has a length selected from the group consisting of at least 6 nucleotides long; at least 8 nucleotides long; at least 10 nucleotides long; at least 12 nucleotides long; and at least 14 nucleotides long.

44. The method of claim 33, wherein the transcription reaction mixture further comprises manganese ions.

45. The method of claim 44, wherein the concentration of magnesium ions is between 3.0 and 3.5 times greater than the concentration of manganese ions.

46. The method of claim 33, wherein each NTP is present at a concentration of 0.5 mM each, the concentration of magnesium ions is 5.0 mM, and the concentration of manganese ions is 1.5 mM.

47. The method of claim 33, wherein each NTP is present at a concentration of 1.0 mM each, the concentration of magnesium ions is 6.5 mM, and the concentration of manganese ions is 2.0 mM.

48. The method of claim 33, wherein each NTP is present at a concentration of 2.0 mM each, the concentration of magnesium ions is 9.6 mM, and the concentration of manganese ions is 2.9 mM.

49. The method of claim 33, wherein the transcription reaction mixture further comprises 2′-OH GTP.

50. The method of claim 33, wherein the transcription reaction mixture further comprises a polyalkylene glycol.

51. The method of claim 50, wherein the polyalkylene glycol is polyethylene glycol (PEG).

52. The method of claim 33, wherein the transcription reaction mixture further comprises GMP.

53. An aptamer composition comprising a sequence where substantially all adenosine nucleotides are 2′-OH adenosine, substantially all guanosine nucleotides are 2′-OH guanosine, substantially all cytidine nucleotides are 2′-O-methyl cytidine, and substantially all uridine nucleotides are 2′-O-methyl uridine.

54. The aptamer composition of claim 53 wherein said aptamer comprises a sequence composition where at least 80% of all adenosine nucleotides are 2′-OH adenosine, at least 80% of all guanosine nucleotides are 2′-OH guanosine, at least 80% of all cytidine nucleotides are 2′-O-methyl cytidine and at least 80% of all uridine nucleotides are 2′-O-methyl uridine.

55. The aptamer composition of claim 53 wherein said aptamer comprises a sequence composition where at least 90% of all adenosine nucleotides are 2′-OH adenosine, at least 90% of all guanosine nucleotides are 2′-OH guanosine, at least 90% of all cytidine nucleotides are 2′-O-methyl cytidine and at least 90% of all uridine nucleotides are 2′-O-methyl uridine.

56. The aptamer composition of claim 53 wherein said aptamer comprises a sequence composition where 100% of all adenosine nucleotides are 2′-OH adenosine, at 100% of all guanosine nucleotides are 2′-OH guanosine, 100% of all cytidine nucleotides are 2′-O-methyl cytidine and 100% of all uridine nucleotides are 2′-O-methyl uridine.

57. An aptamer composition comprising a sequence where substantially all purine nucleotides are 2′-deoxy purines and substantially all pyrimidine nucleotides are 2′-O-methyl pyrimidines.

58. The aptamer composition of claim 57 wherein said aptamer comprises a sequence composition where at least 80% of all purine nucleotides are 2′-deoxy purines and at least 80% of all pyrimidine nucleotides are 2′-O-methylpyrimidines.

59. The aptamer composition of claim 57 wherein said aptamer comprises a sequence composition where at least 90% of all purine nucleotides are 2′-deoxy purines and at least 90% of all pyrimidine nucleotides are 2′-O-methylpyrimidines.

60. The aptamer composition of claim 57 wherein said aptamer comprises a sequence composition where 100% of all purine nucleotides are 2′-deoxy purines and 100% of all pyrimidine nucleotides are 2′-O-methyl pyrimidines

61. An aptamer composition comprising a sequence composition where substantially all guanosine nucleotides are 2′-OH guanosine, substantially all cytidine nucleotides are 2′-O-methyl cytidine, substantially all uridine nucleotides are 2′-O-methyl uridine, and substantially all adenosine nucleotides are 2′-O-methyl adenosine.

62. The aptamer composition of claim 61 wherein said aptamer comprises a sequence composition where at least 80% of all guanosine nucleotides are 2′-OH guanosine, at least 80% of all cytidine nucleotides are 2′-O-methyl cytidine, at least 80% of all uridine nucleotides are 2′-O-methyl uridine, and at least 80% of all adenosine nucleotides are 2′-O-methyl adenosine.

63. The aptamer composition of claim 61 wherein said aptamer comprises a sequence composition where at least 90% of all guanosine nucleotides are 2′-OH guanosine, at least 90% of all cytidine nucleotides are 2′-O-methyl cytidine, at least 90% of all uridine nucleotides are 2′-O-methyl uridine, and at least 90% of all adenosine nucleotides are 2′-O-methyl adenosine.

64. The aptamer composition of claim 61 wherein said aptamer comprises a sequence composition where 100% of all guanosine nucleotides are 2′-OH guanosine, 100% of all cytidine nucleotides are 2′-O-methyl cytidine, 100% of all uridine nucleotides are 2′-O-methyl uridine, and 100% of all adenosine nucleotides are 2′-O-methyl adenosine.

65. An aptamer composition comprising a sequence where substantially all adenosine nucleotides are 2′-O-methyl adenosine, substantially all cytidine nucleotides are 2′-O-methyl cytidine, substantially all guanosine nucleotides are 2′-O-methyl guanosine or deoxy guanosine, substantially all uridine nucleotides are 2′-O-methyl uridine, wherein less than about 10% of the guanosine nucleotides are deoxy guanosine.

66. The aptamer composition of claim 65 wherein said aptamer comprises a sequence composition where at least 80% of all adenosine nucleotides are 2′-O-methyl adenosine, at least 80% of all cytidine nucleotides are 2′-O-methyl cytidine, at least 80% of all guanosine nucleotides are 2′-O-methyl guanosine, at least 80% of all uridine nucleotides are 2′-O-methyl uridine, and no more than about 10% of all guanosine nucleotides are deoxy guanosine.

67. The aptamer composition of claim 65 wherein said aptamer comprises a sequence composition where at least 90% of all adenosine nucleotides are 2′-O-methyl adenosine, at least 90% of all cytidine nucleotides are 2′-O-methyl cytidine, at least 90% of all guanosine nucleotides are 2′-O-methyl guanosine, at least 90% of all uridine nucleotides are 2′-O-methyl uridine, and no more than about 10% of all guanosine nucleotides are deoxy guanosine.

68. The aptamer composition of claim 65 wherein said aptamer comprises a sequence composition where 100% of all adenosine nucleotides are 2′-O-methyl adenosine, 100% of all cytidine nucleotides are 2′-O-methyl cytidine, 90% of all guanosine nucleotides are 2′-O-methyl guanosine, and 100% of all uridine nucleotides are 2′-O-methyl uridine and no more than about 10% of all guanosine nucleotides are deoxy guanosine.

69. An aptamer composition comprising a sequence where substantially all adenosine nucleotides are 2′-O-methyl adenosine, substantially all uridine nucleotides are 2′-O-methyl uridine, substantially all cytidine nucleotides are 2′-O-methyl cytidine, and substantially all guanosine nucleotides are 2′-F guanosine sequence.

70. The aptamer composition of claim 69 wherein said aptamer comprises a sequence composition where at least 80% of all adenosine nucleotides are 2′-O-methyl adenosine, at least 80% of all uridine nucleotides are 2′-O-methyl uridine, at least 80% of all cytidine nucleotides are 2′-O-methyl cytidine, and at least 80% of all guanosine nucleotides are 2′-F guanosine.

71. The aptamer composition of claim 69 wherein said aptamer comprises a sequence composition where at least 90% of all adenosine nucleotides are 2′-O-methyl adenosine, at least 90% of all uridine nucleotides are 2′-O-methyl uridine, at least 90% of all cytidine nucleotides are 2′-O-methyl cytidine, and at least 90% of all guanosine nucleotides are 2′-F guanosine

72. The aptamer composition of claim 69 wherein said aptamer comprises a sequence composition where 100% of all adenosine nucleotides are 2′-O-methyl adenosine, 100% of all uridine nucleotides are 2′-O-methyl uridine, 100% of all cytidine nucleotides are 2′-O-methyl cytidine, and 100% of all guanosine nucleotides are 2′-F guanosine.

73. An aptamer composition comprising a sequence where substantially all adenosine nucleotides are 2′-deoxy adenosine, substantially all cytidine nucleotides are 2′-O-methyl cytidine, substantially all guanosine nucleotides are 2′-O-methyl guanosine, and substantially all uridine nucleotides are 2′-O-methyl uridine.

74. The aptamer composition of claim 73 wherein said aptamer comprises a sequence composition where at least 80% of all adenosine nucleotides are 2′-deoxy adenosine, at least 80% of all cytidine nucleotides are 2′-O-methyl cytidine, at least 80% of all guanosine nucleotides are 2′-O-methyl guanosine, and at least 80% of all uridine nucleotides are 2′-O-methyl uridine.

75. The aptamer composition of claim 73 wherein said aptamer comprises a sequence composition where at least 90% of all adenosine nucleotides are 2′-deoxy adenosine, at least 90% of all cytidine nucleotides are 2′-O-methyl cytidine, at least 90% of all guanosine nucleotides are 2′-O-methyl guanosine, and at least 90% of all uridine nucleotides are 2′-O-methyl uridine.

76. The aptamer composition of claim 73 wherein said aptamer comprises a sequence composition where 100% of all adenosine nucleotides are 2′-deoxy adenosine, 100% of all cytidine nucleotides are 2′-O-methyl cytidine, 100% of all guanosine nucleotides are 2′-O-methyl guanosine, and 100% of all uridine nucleotides are 2′-O-methyl uridine.


SEQUENCE LISTING (468 sequences)

SEQ ID NO: 1 - DNA, 93 bp, Artificial
  aptamer library template ARC254
catcgatgctagtcgtaacgatccnnnnnnnnnnnnnnnnnnnnnnnnnnnnnncgagaacgttctctcctctccctatagtgagtcgtatta

SEQ ID NO: 2 - DNA, 92 bp, Artificial
  aptamer library template ARC255
catgcatcgcgactgactagccgnnnnnnnnnnnnnnnnnnnnnnnnnnnnnngtagaacgttctctcctctccctatagtgagtcgtatta

SEQ ID NO: 3 - DNA, 77 bp, Artificial
  clone of aptamer
gggagaggagagaacgttctcgaaatgatgcatgttcgtaaaatggcagtattggatcgttacaactagcatcgatg

SEQ ID NO: 4 - DNA, 76 bp, Artificial
  clone of aptamer
gggagaggagagaacgttctcgtgccgaggtccggaaccttgatgattggcgggatcgttacgactagcatcgatg

SEQ ID NO: 5 - DNA, 76 bp, Artificial
  clone of aptamer
gggagaggagagaacgttctcgcatttgggctagttgtgaaatggcagtattggatcgttacgactagcatcgatg

SEQ ID NO: 6 - DNA, 76 bp, Artificial
  clone of aptamer
gggagaggagagaacgttctcgaatcgtagatagtcgtgaaatggcagtattggatcgttacgactagcatcgatg

SEQ ID NO: 7 - DNA, 76 bp, Artificial
  clone of aptamer
gggagaggagagaacgttctcgttctagtcggtacgatatgttgacgaatccggatcgttacgactagcatcgatg

SEQ ID NO: 8 - DNA, 78 bp, Artificial
  clone of aptamer
gggagaggagagaacgttctcgtttgatgaggcggacataatccgtgccgagcgggatcgttacgactagcatcgatg

SEQ ID NO: 9 - DNA, 77 bp, Artificial
  clone of aptamer
gggagaggagagaacgttctcgaaggaaaagagtttagtattggccgtccgtgggatcgttacgactagcatcgatg

SEQ ID NO: 10 - DNA, 76 bp, Artificial; identical to SEQ ID NO: 4
  clone of aptamer

SEQ ID NO: 11 - DNA, 76 bp, Artificial
  clone of aptamer
gggagaggagagaacgttctcgtacggtccattgagtttgagatgtcgccatggatcgttacgactagcatcgatg

SEQ ID NO: 12 - DNA, 77 bp, Artificial
  clone of aptamer
gggagaggagagaacgttctcgagttagtggtaactgatatgttgaattgtccggatcgttacgactagcatcgatg

SEQ ID NO: 13 - DNA, 76 bp, Artificial
  clone of aptamer
gggagaggagagaacgttctcgcacggatggcgagaacagagattgctaggtggatcgttacgactagcatcgatg

SEQ ID NO: 14 - DNA, 76 bp, Artificial
  clone of aptamer
gggagaggagagaacgttctcgntancgntncgccntgctaacgcntanttgggatcgttacgactagcatcgatg

SEQ ID NO: 15 - DNA, 77 bp, Artificial
  clone of aptamer
gggagaggagagaacgttctcgaagatgagttttgtcgtgaaatggcagtattggatcgttacgactagcatcgatg

SEQ ID NO: 16 - DNA, 76 bp, Artificial
  clone of aptamer
gggagaggagagaacgttctcgggatgccggattgatttctgatgggtactgggatcgttacgactagcatcgatg

SEQ ID NO: 17 - DNA, 76 bp, Artificial
  clone of aptamer
gggagaggagagaacgttctcgaatggaatgcatgtccatcgctagcatttgggatcgttacgactagcatcgatg

SEQ ID NO: 18 - DNA, 76 bp, Artificial
  clone of aptamer
gggagaggagagaacgttctcgtgctgaggtccggaaccttgatgattggcgggatcgttncnactagcatcgatg

SEQ ID NO: 19 - DNA, 76 bp, Artificial
  clone of aptamer
gggagaggagagaacgttctcgctaattgctgagtcgtgaagtggcagtattggatcgttacgactagcatcgatg

SEQ ID NO: 20 - DNA, 76 bp, Artificial
  clone of aptamer
gggagaggagagaacgttctcgtaacgatgtccggggcgaaaggctagcatgggatcgttacgactagcatcgatg

SEQ ID NO: 21 - DNA, 77 bp, Artificial
  clone of aptamer
gggagaggagagaacgttctcgatgcgattgtcgagatttgtaagatagctgtggatcgttacgactagcatcgatg

SEQ ID NO: 22 - DNA, 76 bp, Artificial
  clone of aptamer
gggagaggagagaacgttctcgcagaaaacatctttgcggttgaatacatgtggatcgttacgactagcatcgatg

SEQ ID NO: 23 - DNA, 76 bp, Artificial
  clone of aptamer
gggagaggagagaacgttctcgaaaaaaganancnnccttcngaatacatgcggatcgttacgactagcatcgatg

SEQ ID NO: 24 - DNA, 76 bp, Artificial
  clone of aptamer
gggagaggagagaacgttctcgagagtgattcgatgcttcangaatacatgtggatcgttacgactagcatcgatg

SEQ ID NO: 25 - DNA, 81 bp, Artificial
  clone of aptamer
gggagaggagagaacgttctcgacannncntngctnggttgantacatgtgnntntcnnnancnntnntctntnanagggg

SEQ ID NO: 26 - DNA, 76 bp, Artificial
  clone of aptamer
gggagaggagagaacgttctcgaagaaggaaagctgcaagtcgaatacacgcggatcgttacgactagcatcgatg

SEQ ID NO: 27 - DNA, 76 bp, Artificial
  clone of aptamer
gggagaggagagaacgttctcgcaaaaacatcgattacagttgagtacatgtggatcgttacgactagcatcgatg

SEQ ID NO: 28 - DNA, 73 bp, Artificial
  clone of aptamer
gggagaggagagaacgttctcgagacatcattgctcgttgaatacatgtggatcgttacgactagcatcgatg

SEQ ID NO: 29 - DNA, 76 bp, Artificial
  clone of aptamer
gggagaggagagaacgttctcgccaaagtagcttcgacagtcgaatacatgtggatcgttacgactagcatcgatg

SEQ ID NO: 30 - DNA, 76 bp, Artificial
  clone of aptamer
gggagaggagagaacgttctcgaaaatcagtactgtgcagtcgaatacatgcggatcgttacgactagcatcgatg

SEQ ID NO: 31 - DNA, 76 bp, Artificial
  clone of aptamer
gggagaggagagaacgttctcgtaatgacatcaatgcttcttgaatacaggtggatcgttacgactagcatcgatg

SEQ ID NO: 32 - DNA, 75 bp, Artificial
  clone of aptamer
gggagaggagagaacgttctcgagaaaaacgatctgtgacgtgtaatccgcggatcgttacgactagcatcgatg

SEQ ID NO: 33 - DNA, 76 bp, Artificial
  clone of aptamer
gggagaggagagaacgttctcgcaacaaacgtcgacgcttctgaatacatgtggatcgttacgactagcatcgatg

SEQ ID NO: 34 - DNA, 76 bp, Artificial
  clone of aptamer
gggagaggagagaacgttctcgtgatcatagaaatgctagctgaatacatgtggatcgttacgactagcatcgatg

SEQ ID NO: 35 - DNA, 75 bp, Artificial
  clone of aptamer
gggagaggagagaacgttctcgcagcgtaaaatgcttttcgaagtacatgtggatcgttacgactagcatcgatg

SEQ ID NO: 36 - DNA, 76 bp, Artificial
  clone of aptamer
gggagaggagagaacgttctcgccaagaatcaatcgcttgtcgaatacatgcggatcgttacgactagcatcgatg

SEQ ID NO: 37 - DNA, 76 bp, Artificial
  clone of aptamer
gggagaggagagaacgttctcgtgatcatagaaatgctagctgagtacatgtggatcgttacgactagcatcgatg

SEQ ID NO: 38 - DNA, 76 bp, Artificial; identical to SEQ ID NO: 22
  clone of aptamer

SEQ ID NO: 39 - DNA, 78 bp, Artificial
  clone of aptamer
gggagaggagagaacgttctcgnaaacanncatctattgnagttgaatacatgtggatcgttacgactagcatcgatg

SEQ ID NO: 40 - DNA, 76 bp, Artificial
  clone of aptamer
gggagaggagagaacgttctcgctaaagattcgctgcttgccgaatacatgtggatcgttacgactagcatcgatg

SEQ ID NO: 41 - DNA, 76 bp, Artificial
  clone of aptamer
gggagaggagagaacgttctcgggttttgtctgcgtttgtgcgttgaacccgggatcgttacgactagcatcgatg

SEQ ID NO: 42 - DNA, 77 bp, Artificial
  clone of aptamer
gggagaggagagaacgttctcgtgattacgtgatgaggatccgcgttttctcgggatcgttacgactagcatcgatg

SEQ ID NO: 43 - DNA, 76 bp, Artificial
  clone of aptamer
gggagaggagagaacgttctcgttagtgaaaacgatcatgcatgtggatcgcggatcgttacgactagcatcgatg

SEQ ID NO: 44 - DNA, 75 bp, Artificial
  clone of aptamer
gggagaggagagaacgttctcgtgttcattcgtttgcttatcgttgcatgtggatcgttacgactagcatcgatg

SEQ ID NO: 45 - DNA, 76 bp, Artificial
  clone of aptamer
aggagaggagagaacgttctcggcagagtgtgatgtgcatccgcacgtgccgggatcgttacgactagcatcgatg

SEQ ID NO: 46 - DNA, 76 bp, Artificial
  clone of aptamer
gggagaggagagaacgttctcgttagtaaatacgatcgtgcatgtggatcgcggatcgttacgactagcatcgatg

SEQ ID NO: 47 - DNA, 77 bp, Artificial
  clone of aptamer
gggagaggagagaacgcccccctgattncgtgaagaggatccgcantttcncgggatcgttacgactagcatcgatg

SEQ ID NO: 48 - DNA, 76 bp, Artificial
  clone of aptamer
gggagaggagagaacgttctcgtggctttggaacgggtacggatttggcacgggatcgttacgactagcatcgatg

SEQ ID NO: 49 - DNA, 77 bp, Artificial; identical to SEQ ID NO: 42
  clone of aptamer

SEQ ID NO: 50 - DNA, 76 bp, Artificial
  clone of aptamer
gggagaggagagaacgttctcgtcattggtgacngcgttgcatgtggatcgcggatcgttacgactagcatcgatg

SEQ ID NO: 51 - DNA, 76 bp, Artificial
  clone of aptamer
gggagaggagagaacgttctcgntggtnnaangcttttgtngggntanntgtggatcgttacgactagcatcgatg

SEQ ID NO: 52 - DNA, 76 bp, Artificial
  clone of aptamer
gggagaggagagaacgttctcgtggctttggaacgaattcggatttggcacgggatcgttacgactagcatcgatg

SEQ ID NO: 53 - DNA, 75 bp, Artificial
  clone of aptamer
gggagaggagagaacgttctcgtgcgatgtcgtggatttccgtttcgcaagggatcgttacgactagcatcgatg

SEQ ID NO: 54 - DNA, 76 bp, Artificial
  clone of aptamer
gggagaggagagaacgttctcgtgaagcagatgtcgttggcgacttagagggggatcgttacgactagcatcgatg

SEQ ID NO: 55 - DNA, 77 bp, Artificial
  clone of aptamer
gggagaggagagaacgttctcgtgatttcgtgatgaggatccgcgttttctcgggatcgttacgactagcatcgatg

SEQ ID NO: 56 - DNA, 75 bp, Artificial
  clone of aptamer
gggagaggagagaacgttctcgctagtaacgatgacttgatgagcatccgaggatcgttacgactagcatcgatg

SEQ ID NO: 57 - DNA, 76 bp, Artificial
  clone of aptamer
gggagaggagagaacgttctcgtcataagtaacgacgttgcatgtggatcgcggatcgttacgactagcatcgatg

SEQ ID NO: 58 - DNA, 76 bp, Artificial
  clone of aptamer
gggagaggagagaacgttctcgcaaggagatggttgctagctgagtacatgtggatcgttacgactagcatcgatg

SEQ ID NO: 59 - DNA, 78 bp, Artificial
  clone of aptamer
gggagaggagagaacgttctcgcgatatgcagtttgagaagtcgcgcattcgggggatcgttacgactagcatcgatg

SEQ ID NO: 60 - DNA, 75 bp, Artificial
  clone of aptamer
gggagaggagagaacgttctcgtgcgacgggcttcttgtgtcattcgcatgggatcgttacgactagcatcgatg

SEQ ID NO: 61 - DNA, 76 bp, Artificial
  clone of aptamer
gggagaggagagaacgttctcggcattgcagttgataggtcgcgcagtgctgggatcgttacgactagcatcgatg

SEQ ID NO: 62 - DNA, 78 bp, Artificial
  clone of aptamer
gggagaggagagaacgttctcgcgatatgcagtctgagaagtcgcgcattcgagggatcgttacgactagcatcgatg

SEQ ID NO: 63 - DNA, 76 bp, Artificial
  clone of aptamer
gggagaggagagaacgttctcgtgtagcaagcatgtggatcgcgactgcacgggatcgttacgactagcatcgatg

SEQ ID NO: 64 - DNA, 76 bp, Artificial
  clone of aptamer
gggagaggagagaacgttctcggataagcagttgagatgtcgcgctttgacgggatcgttacgactagcatcgatg

SEQ ID NO: 65 - DNA, 75 bp, Artificial
  clone of aptamer
gggagaggagagaacgttctcgatgancantttgagaagtcgcgcttgtcgggatcgttacgactagcatcgatg

SEQ ID NO: 66 - DNA, 75 bp, Artificial
  clone of aptamer
gggagaggagagaacgttctcgagtaatgcagtggaagtcgcgcattacctgggatcgttacgactagcatcatg

SEQ ID NO: 67 - DNA, 78 bp, Artificial; identical to SEQ ID NO: 59
  clone of aptamer

SEQ ID NO: 68 - DNA, 73 bp, Artificial
  clone of aptamer
gggagaggagagaacgttctcgtgatncagttganaagtcncgcatacaggatcgttacgactagcatcgatg

SEQ ID NO: 69 - DNA, 76 bp, Artificial
  clone of aptamer
gggagaggagagaacgttctcgagtaatgctgtggaagtcgcgcatttcctgggatcgttacgactagcatcgatg

SEQ ID NO: 70 - DNA, 76 bp, Artificial; identical to SEQ ID NO: 61
  clone of aptamer

SEQ ID NO: 71 - DNA, 78 bp, Artificial
  clone of aptamer
gggagaggagagaacgttctcgcgatatgcagtttgggaagtcgcgcattcgagggatcgttacgactagcatcgatg

SEQ ID NO: 72 - DNA, 78 bp, Artificial
  clone of aptamer
gggagaggagagaacgttctcgcnatatgctgtttganaantcgcgcattcgggggatcgttacgactagcatcgatg

SEQ ID NO: 73 - DNA, 78 bp, Artificial
  clone of aptamer
gggagaggagagaacgttctcgcgtagattgggctgaatgggatatctttagcgggatcgttacgactagcatcgatg

SEQ ID NO: 74 - DNA, 78 bp, Artificial
  clone of aptamer
gggagaggagagaacgttctcgcgatatgcagtttgagaagtcgcgctttcgagggatcgttacgactagcatcgatg

SEQ ID NO: 75 - DNA, 78 bp, Artificial
  clone of aptamer
gggagaggagagaacgttctcgtcaatctgatgtagcctcacgtgggcggagtcggatcgttacgactagcatcgatg

SEQ ID NO: 76 - DNA, 45 bp, Artificial
  clone of aptamer
gggagaggagagaacgttctcggatcgttacgactagcatcgatg

SEQ ID NO: 77 - DNA, 45 bp, Artificial; identical to SEQ ID NO: 76
  clone of aptamer

SEQ ID NO: 78 - DNA, 76 bp, Artificial
  clone of aptamer
gggagaggagagaacgttctcggtggtgttgctgaactgtcgcgtttcgccgggatcgttacgactagcatcgatg

SEQ ID NO: 79 - DNA, 77 bp, Artificial
  clone of aptamer
gggagaggagagaacgttctcgtcgcgattgcatattttccgccttgctgtgaggatcgttacgactagcatcgatg

SEQ ID NO: 80 - DNA, 78 bp, Artificial
  clone of aptamer
gggagaggagagaacgttctcgcgatttgcagtttgagatgtcgcgcattcgagggatcgttacgactagcatcgatg

SEQ ID NO: 81 - DNA, 78 bp, Artificial; identical to SEQ ID NO: 59
  clone of aptamer

SEQ ID NO: 82 - DNA, 76 bp, Artificial
  clone of aptamer
gggagaggagagaacgttctcgttggtgcagtttgagatgtcgcgcaccttgggatcgttacgactagcatcgatg

SEQ ID NO: 83 - DNA, 80 bp, Artificial
  clone of aptamer
gggagaggagagaacgttctcggtattggttccattaagctggacactctgctccgggatcgttacgactagcatcgatg

SEQ ID NO: 84 - DNA, 76 bp, Artificial
  clone of aptamer
gggagaggagagaacgttctcgttggtgcagtttgagatgtcgcgcgccttgggatcgttacgactagcatcgatg

SEQ ID NO: 85 - DNA, 78 bp, Artificial
  clone of aptamer
gggagaggagagaacgttctcgcgatatgcagtttgagaagtcgcgcattcgagggatcgttacnactagcatcgatg

SEQ ID NO: 86 - DNA, 78 bp, Artificial; identical to SEQ ID NO: 59
  clone of aptamer

SEQ ID NO: 87 - DNA, 80 bp, Artificial
  clone of aptamer
gggagaggagagaacgctctcggggacnnaaanncgaattgncgcgtgngtccgggggagcgcccgactagtcatcgatg

SEQ ID NO: 88 - DNA, 78 bp, Artificial
  clone of aptamer
gggagaggagagaacgttctcgcgatatgnantttgagaagtcgcgcattcgggggatcgttacgactagcatcgatg

SEQ ID NO: 89 - DNA, 75 bp, Artificial
  clone of aptamer
gggagaggagagaacgttctcggtgtacagcttgagatgtcgcgtactccgggatcgttacgactagcatcgatg

SEQ ID NO: 90 - DNA, 78 bp, Artificial; identical to SEQ ID NO: 59
  clone of aptamer

SEQ ID NO: 91 - DNA, 76 bp, Artificial
  clone of aptamer
gggagaggagagaacgttctcgagtaagaaagctgaatggtcgcacttctcgggatcgttacgactagcatcgatg

SEQ ID NO: 92 - DNA, 78 bp, Artificial
  clone of aptamer
agggagaggaagaacgttctcgcgatgtgcagtttgagaagtcgcgcattcgagggatcgttacgactagcatcgatg

SEQ ID NO: 93 - DNA, 76 bp, Artificial
  clone of aptamer
gggagaggagagaacgttctcgaaagaatcagcatgcggatcgcggctttcgggatcgttacgactagcatcgatg

SEQ ID NO: 94 - DNA, 79 bp, Artificial
  clone of aptamer
gggagaggagagaacgttctcgantccantntncntggaggagtaagtacctgagggatcgttacgactagcatcgatg

SEQ ID NO: 95 - DNA, 76 bp, Artificial
  clone of aptamer
gggagaggagagaacgttctcgggaaacaaggaacttagagttanttgaccgggatcgttacgactagcatcgatg

SEQ ID NO: 96 - DNA, 76 bp, Artificial
  clone of aptamer
gggagaggagagaacgttctcgtaccatgcaaggaacataatagttagcgtgggatcgttacgactagcatcgatg

SEQ ID NO: 97 - DNA, 76 bp, Artificial
  clone of aptamer
gggagaggagagaacgttctcgggacacaaggaacacaatagttagtgtacgggatcgttacgactagcatcgatg

SEQ ID NO: 98 - DNA, 76 bp, Artificial
  clone of aptamer
gggagaggagagaacgttctcgtctgcaaggaacacaatagttagcattgcgggatcgttacgactagcatcgatg

SEQ ID NO: 99 - DNA, 76 bp, Artificial
  clone of aptamer
gggagaggagagaacgttctcgcgccaacaaagctggagtacttagagcgcgggatcgttacgactagcatcgatg

SEQ ID NO: 100 - DNA, 76 bp, Artificial
  clone of aptamer
gggagaggagagaacgttctcgattgcaaaatagctgtagaactaagcaatcggatcgttacgactagcatcgatg

SEQ ID NO: 101 - DNA, 76 bp, Artificial
  clone of aptamer
gggagaggagagaacgttctcgtgagatgactatgttaagatgacgctgttgggatcgttacgactagcatcgatg

SEQ ID NO: 102 - DNA, 76 bp, Artificial
  clone of aptamer
gggagaggagagaacgttctcggganacaaggaacncaatatttagtgaacgggatcgttacgactagcatcgatg

SEQ ID NO: 103 - DNA, 76 bp, Artificial
  clone of aptamer
gggagaggagagaacgttctcgccaaggaacacaatagttaggtgagaatcgggatcgttacgactagcatcgatg

SEQ ID NO: 104 - DNA, 75 bp, Artificial
  clone of aptamer
gggagaggagagaacgttctcggtacaaggaacacaatagttagtgccgtgggatcgttacgactagcatcgatg

SEQ ID NO: 105 - DNA, 77 bp, Artificial
  clone of aptamer
gggagaggagagaacgttctcgattcaacggtccaaaaaagctgtagtacttaggatcgttacgactagcatcgatg

SEQ ID NO: 106 - DNA, 76 bp, Artificial
  clone of aptamer
gggagaggagagaacgttctcgcaatgcaaggaacacaatagttagcagccgggatcgttacgactagcatcgatg

SEQ ID NO: 107 - DNA, 76 bp, Artificial
  clone of aptamer
gggagaggagagaacgttctcgaaaggagaaagctgaagtacttactatgcgggatcgttacgactagcatcgatg

SEQ ID NO: 108 - DNA, 76 bp, Artificial
  clone of aptamer
gggagaggagagaacgttctcgcacaaggaacacaatagttagtgcaagacgggatcgttacgactagcatcgatg

SEQ ID NO: 109 - DNA, 76 bp, Artificial
  clone of aptamer
gggagaggagagaacgttctcgcacaaggaactacgagttagtgtgggagtgggatcgttacgactagcatcgatg

SEQ ID NO: 110 - DNA, 76 bp, Artificial
  clone of aptamer
gggagaggagagaacgttctcgcacaaggaacacaatagttagtgcaagacgggatcgttacgactagcatcgata

SEQ ID NO: 111 - DNA, 75 bp, Artificial
  clone of aptamer
gggagaggagagaacgttctcggcgggaaaatagctgtagtactaacccacggatcgttacgactagcatcgatg

SEQ ID NO: 112 - DNA, 76 bp, Artificial
  clone of aptamer
gggagaggagagaacgttctcggcctcaaggaaaagaaaatttagaggcccgggatcgttacgactagcatcgatg

SEQ ID NO: 113 - DNA, 76 bp, Artificial
  clone of aptamer
gggagaggagagaacgttctcggaacaagatagctgaaggactaagtttacgggatcgttacgactagcatcgatg

SEQ ID NO: 114 - DNA, 76 bp, Artificial; identical to SEQ ID NO: 113
  clone of aptamer

SEQ ID NO: 115 - DNA, 77 bp, Artificial
  clone of aptamer
gggagaggagagaacgttctcggagccaaggaaacgaagatttaggctcattgggatcgttacgactagcatcgatg

SEQ ID NO: 116 - DNA, 76 bp, Artificial
  clone of aptamer
gggagaggagagaacgttctcgatcacaagaaatgtggganggtagtgatncnnntcgttncgactagcatcgatg

SEQ ID NO: 117 - DNA, 76 bp, Artificial
  clone of aptamer
gggagaggagagaacgttctcgtcgaaagggagctttgtctcgggacagaacggatcgttacgactagcatcgatg

SEQ ID NO: 118 - DNA, 76 bp, Artificial
  clone of aptamer
gggagaggagagaacgntctcgtgcaaagatagctggaggactaatgcggcgggatcgttacgactagcatcgatg

SEQ ID NO: 119 - DNA, 76 bp, Artificial; identical to SEQ ID NO: 117
  clone of aptamer

SEQ ID NO: 120 - DNA, 76 bp, Artificial
  clone of aptamer
gggagaggagagaacgttctcgncnaaggngagctttgtcccnggacanaangnatcgttacaactagcatcgatg

SEQ ID NO: 121 - DNA, 76 bp, Artificial; identical to SEQ ID NO: 113
  clone of aptamer

SEQ ID NO: 122 - DNA, 76 bp, Artificial; identical to SEQ ID NO: 113
  clone of aptamer

SEQ ID NO: 123 - DNA, 78 bp, Artificial
  clone of aptamer
gggagaggagagaacgttctcggcgcaaaaaaagctggagtacttagtgtcgagggatcgttacgactagcatcgatg

SEQ ID NO: 124 - DNA, 76 bp, Artificial; identical to SEQ ID NO: 117
  clone of aptamer

SEQ ID NO: 125 - DNA, 76 bp, Artificial
  clone of aptamer
gggagaggagagaacgttctcgacacaagaaagctgcagaacttagggtcgtggatcgttacgactagcatcgatg

SEQ ID NO: 126 - DNA, 76 bp, Artificial
  clone of aptamer
gggagaggagagaacgttctcggaacnggattgttgaaggactaantttacgggatcgttacgactagcatcgatg

SEQ ID NO: 127 - DNA, 76 bp, Artificial
  clone of aptamer
gggagaggagagaacgttctcggcctcaagggaaagaaaatttagaggcccgggatcgttacgactagcatcgatg

SEQ ID NO: 128 - DNA, 77 bp, Artificial
  clone of aptamer
gggagaggagagaacgttctcggaaacaagcttagaaattcgcacccttgccgggatcgttacgactagcatcgatg

SEQ ID NO: 129 - DNA, 75 bp, Artificial
  clone of aptamer
gggagaggagagaacgttctcgaaagaaaaaagctggagaacttacttccgggatcgttacgactagcatcgatg

SEQ ID NO: 130 - DNA, 78 bp, Artificial
  clone of aptamer
gggagaggagagaacgttctcggtgattgtactcacatagaaatggcaacactgggatcgttacgactagcatcgatg

SEQ ID NO: 131 - DNA, 76 bp, Artificial
  clone of aptamer
gggagaggagagaacgttctcgggttcaaggaacatgatagttagaacccgcggatcgttacgactagcatcgatg

SEQ ID NO: 132 - DNA, 77 bp, Artificial
  clone of aptamer
gggagaggagagaacgttctcgttccgaaaggaacacaatagttatcggattgggatcgttacgactagcatcgatg

SEQ ID NO: 133 - DNA, 76 bp, Artificial; identical to SEQ ID NO: 98
  clone of aptamer

SEQ ID NO: 134 - DNA, 74 bp, Artificial
  clone of aptamer
gggagaggagagaacgttctcggtacaaggaacacaatagttagtgccggggatcgttacgactagcatcgatg

SEQ ID NO: 135 - DNA, 75 bp, Artificial
  clone of aptamer
gggagaggagagaacgttctcggaactcagagatcctatgtggaccagagaggatcgttacgactagcatcgatg

SEQ ID NO: 136 - DNA, 76 bp, Artificial
  clone of aptamer
gggagaggagagaacgttctcgctgagcaaggaacgtaatagttagcctgcgggatcgttacgactagcatcgatg

SEQ ID NO: 137 - DNA, 77 bp, Artificial
  clone of aptamer
gggagaggagagaacgttctcgnannnataaatgatggatcncttattgtnnaggatcgttacgactagcatcgatg

SEQ ID NO: 138 - DNA, 74 bp, Artificial
  clone of aptamer
gggagaggagagaacgttctcggcttggaaaaatagcttttgggcatccgggatcgttacgactagcatcgatg

SEQ ID NO: 139 - DNA, 76 bp, Artificial
  clone of aptamer
gggagaggagagaacgttctcgggttcaaggaacatgatagctagaacccgcggatcgttacgactagcatcgatg

SEQ ID NO: 140 - DNA, 76 bp, Artificial; identical to SEQ ID NO: 131
  clone of aptamer

SEQ ID NO: 141 - DNA, 76 bp, Artificial
  clone of aptamer
gggagaggagagaacgttctcgtgggcagggaacacaatagttagcctacgcggatcgttacgactagcatcgatg

SEQ ID NO: 142 - DNA, 75 bp, Artificial
  clone of aptamer
gggagaggagagaacgttctcgcgtgaaaggaacacaatagttatcgtgcgggatcgttacgactagcatcgatg

SEQ ID NO: 143 - DNA, 77 bp, Artificial
  clone of aptamer
gggagaggagagaacgttctcgcgaggtttatcctagacgactaaccgcctggggatcgttacgactagcatcgatg

SEQ ID NO: 144 - DNA, 76 bp, Artificial
  clone of aptamer
gggagaggagagaacgttctcgtctgctaggaacacaatagttagcattgcgggatcgttacgactagcatcgatg

SEQ ID NO: 145 - DNA, 77 bp, Artificial
  clone of aptamer
gggagaggagagaacgttctcgcacaaggaactacgagttagtgtgggagtggggatcgttacgactagcatcgatg

SEQ ID NO: 146 - DNA, 77 bp, Artificial
  clone of aptamer
gggagaggagagaacgttctcgtgacacgaggaacttagagttagtagcacgaggatcgttacgactagcatcgatg

SEQ ID NO: 147 - DNA, 76 bp, Artificial
  clone of aptamer
gggagaggagagaacgttctcggcggcgaaggaacacaatagttacgtcccgggatcgttacgactagcatcgatg

SEQ ID NO: 148 - DNA, 76 bp, Artificial
  clone of aptamer
gggagaggagagaacgttctcgagcccaaaaaagctgaagtactttgggcagggatcgttacgactagcatcgatg

SEQ ID NO: 149 - DNA, 75 bp, Artificial; identical to SEQ ID NO: 104
  clone of aptamer

SEQ ID NO: 150 - DNA, 45 bp, Artificial; identical to SEQ ID NO: 76
  clone of aptamer

SEQ ID NO: 151 - DNA, 76 bp, Artificial
  clone of aptamer
gggagaggagagaacgttctcgtgcgcaaggaacacaatagttagggcgcgaggatcgttacgactagcattgatg

SEQ ID NO: 152 - DNA, 76 bp, Artificial
  clone of aptamer
gggagaggagagaacgttctcggaatggaaggaacacaatagttaccagacgggatcgttacgactagcatcgatg

SEQ ID NO: 153 - DNA, 76 bp, Artificial; identical to SEQ ID NO: 98
  clone of aptamer

SEQ ID NO: 154 - DNA, 76 bp, Artificial
  clone of aptamer
gggagaggagagaacgttctcgagacaagacagctggaggactaagtcacgaggatcgttacgactagcatcgatg

SEQ ID NO: 155 - DNA, 76 bp, Artificial
  clone of aptamer
gggagaggagagaacgttctcgatgcccgcaaaggaacacgatagttatgcgggatcgttacgactagcatcgatg

SEQ ID NO: 156 - DNA, 76 bp, Artificial
  clone of aptamer
gggagaggagagaacgttctcgtctgnnaggaacacaatatttagcattgcgggatcgttacgactagcatcgatg

SEQ ID NO: 157 - DNA, 76 bp, Artificial
  clone of aptamer
gggagaggagagaacgttctcgaatgtgcggagcagtattggtacactttcgggatcgttacgactagcatcgatg

SEQ ID NO: 158 - DNA, 76 bp, Artificial; identical to SEQ ID NO: 103
  clone of aptamer

SEQ ID NO: 159 - DNA, 76 bp, Artificial; identical to SEQ ID NO: 103
  clone of aptamer

SEQ ID NO: 160 - DNA, 76 bp, Artificial
  clone of aptamer
gggagaggagagaacgttctcgggaagcaaggaacttagagttagttgaccgggatcgttacgactagcatcgatg

SEQ ID NO: 161 - DNA, 76 bp, Artificial
  clone of aptamer
gggagaggagagaacgttctcgtgggcaaggaacacaatagttagcctacgcggatcgttacgactagcatcgatg

SEQ ID NO: 162 - DNA, 76 bp, Artificial
  clone of aptamer
gggagaggagagaacgttctcgtcgggcatggaacacaatagttagaccgcgggatcgttacgactagcatcgatg

SEQ ID NO: 163 - DNA, 75 bp, Artificial
  clone of aptamer
gggagaggagagaacgttctcggtcgcaaggaacataatagttagcggaggggatcgttacgactagcatcgatg

SEQ ID NO: 164 - DNA, 76 bp, Artificial; identical to SEQ ID NO: 98
  clone of aptamer

SEQ ID NO: 165 - DNA, 77 bp, Artificial
  clone of aptamer
gggagaggagagaacgttctcgccgacaatcagctcggatcgtgtgctacgctggatcgttacgactagcatcgatg

SEQ ID NO: 166 - DNA, 77 bp, Artificial
  clone of aptamer
gggagaggagagaacgttctcgagacaagatagctgaaggactaagtcacgagggatcgttacgactagcatcgatg

SEQ ID NO: 167 - DNA, 76 bp, Artificial
  clone of aptamer
gggagaggagagaacgttctcggaacaagatagctgaaggactaagtttgcgggatcgttacgactagcatcgatg

SEQ ID NO: 168 - DNA, 77 bp, Artificial
  clone of aptamer
gggagaggagagaacgttctcggagncaaggaaacnaatatttaggctcantggnnncnttncanctagcnncnnta

SEQ ID NO: 169 - DNA, 76 bp, Artificial; identical to SEQ ID NO: 98
  clone of aptamer

SEQ ID NO: 170 - DNA, 76 bp, Artificial; identical to SEQ ID NO: 113
  clone of aptamer

SEQ ID NO: 171 - DNA, 45 bp, Artificial; identical to SEQ ID NO: 76
  clone of aptamer

SEQ ID NO: 172 - DNA, 78 bp, Artificial
  clone of aptamer
gggagaggagagaacgttctcggtgatagtactcacatagaaatggctacactgggatcgttacgactagcatcgatg

SEQ ID NO: 173 - DNA, 76 bp, Artificial
  clone of aptamer
gggagaggagagaacgttctcgcctgggcaaggaacagaaaagttagcgccaggatcgttacgactagcatcgatg

SEQ ID NO: 174 - DNA, 76 bp, Artificial
  clone of aptamer
gggagaggagagaacgttctcgtaacggacaaaaggaaccgggaagttatctggatcgttacgactagcatcgatg

SEQ ID NO: 175 - DNA, 76 bp, Artificial
  clone of aptamer
gggagaggagagaacgttctcgcgcacaagatagagaagactaagtccgcggggatcgttacgactagcatcgatg

SEQ ID NO: 176 - DNA, 76 bp, Artificial
  clone of aptamer
gggagaggagagaacgttctcgcgcacaagatagagaagactaagttcgcggggatcgttacgactagcatcgatg

SEQ ID NO: 177 - DNA, 76 bp, Artificial
  clone of aptamer
gggagaggagagaacgttctcgcgccaataaagctggagtacttagagcgcgggatcgttacgactagcatcgatg

SEQ ID NO: 178 - DNA, 76 bp, Artificial
  clone of aptamer
gggagaggagagaacgttctcgggaaacaaggaacttagagttagttgaccgggatcgttacgactagcatcgatg

SEQ ID NO: 179 - DNA, 76 bp, Artificial
  clone of aptamer
gggagaggagagaacgttctcgctagcaagataggtgggactaagctagtgaggatcgttacgactagcatcgatg

SEQ ID NO: 180 - DNA, 76 bp, Artificial
  clone of aptamer
gggagaggagagaacgttctcgtcgaaggggagctttgtctcgggacagaacggatcgttacgactagcatcgatg

SEQ ID NO: 181 - DNA, 76 bp, Artificial; identical to SEQ ID NO: 113
  clone of aptamer

SEQ ID NO: 182 - DNA, 76 bp, Artificial; identical to SEQ ID NO: 167
  clone of aptamer

SEQ ID NO: 183 - DNA, 77 bp, Artificial
  clone of aptamer
gggagaggaganntccccncncggaaaaanaaaaaagaagaantangttngggggatcgttacgactagcatcgatg

SEQ ID NO: 184 - RNA, 30 nt, Artificial
  r/mGmH aptamer ARC224 -Stabilized VEGF Aptamer
cgnunugcnguuugngnngucgcgcnuucg

SEQ ID NO: 185 - RNA, 30 nt, Artificial; identical to SEQ ID NO: 184
  r/mGmH aptamer ARC225 - Stabilized VEGF Aptamer

SEQ ID NO: 186 - RNA, 24 nt, Artificial
  r/mGmH aptamer ARC226 Single-hydroxy VEGF aptamer
gnucnugcnuguggnucgcggnuc

SEQ ID NO: 187 - RNA, 23 nt, Artificial
  r/mGmH aptamer ARC245 VEGF Aptamer
nugcnguuugngnngucgcgcnu

SEQ ID NO: 188 - RNA, 23 nt, Artificial
  r/mGmH aptamer ARC259 hVEGF Aptamer
ncgcnguuugngnngucgcgcgu

SEQ ID NO: 189 - DNA, 82 bp, Artificial
  clone of aptamer
gggagaggagagaacgttctactatgaagggttttaaagatgacacattagccgctgtcgatcgatcgatcgatgaagggcg

SEQ ID NO: 190 - DNA, 75 bp, Artificial
  clone of aptamer
gggagaggagagaacgttctacaggcagttctggggacccatgggggaagtgcgctgtcgatcgatcgatcgatg

SEQ ID NO: 191 - DNA, 75 bp, Artificial
  clone of aptamer
gggagaggagagaacgttctacgattagcagggagggagagtgcgaagaggacgctgtcgatcgatcgatcgatg

SEQ ID NO: 192 - DNA, 75 bp, Artificial
  clone of aptamer
gggagaggagagaacgttctacactctggggacccgtgggggagtgcagcaacgctgtcgatcgatcgatcgatg

SEQ ID NO: 193 - DNA, 75 bp, Artificial
  clone of aptamer
gggagaggagagaacgttctacaagcagttctggggacccatgggggaagtgcgctgtcgatcgatcgatcgatg

SEQ ID NO: 194 - DNA, 74 bp, Artificial
  clone of aptamer
gggagaggagagaacgttctacgaggtgagggtctacaatggagggatggtcgctgtcgatcgatcgatcgatg

SEQ ID NO: 195 - DNA, 75 bp, Artificial
  clone of aptamer
gggagaggagagaacgttctacccgcagcatagcctgnggacccatgnggggcgctgtcgatcgatcgatcgatg

SEQ ID NO: 196 - DNA, 75 bp, Artificial
  clone of aptamer
gggagaggagagaacgttctactggggggcgtgttcattagcagcgtcgtgtcgctgtcgatcgatcgatcgatg

SEQ ID NO: 197 - DNA, 75 bp, Artificial; identical to SEQ ID NO: 190
  clone of aptamer

SEQ ID NO: 198 - DNA, 75 bp, Artificial
  clone of aptamer
gggagaggagagaacgttctacgcagcgcatctggggacccaagaggggattcgctgtcgatcgatcgatcgatg

SEQ ID NO: 199 - DNA, 75 bp, Artificial; identical to SEQ ID NO: 190
  clone of aptamer

SEQ ID NO: 200 - DNA, 73 bp, Artificial
  clone of aptamer
gggagaggagagaacgttctacgggatgggtagttggatggaaatgggaacgctgtcgatcgatcgatcgatg

SEQ ID NO: 201 - DNA, 74 bp, Artificial
  clone of aptamer
gggagaggagagaacgttctacgaggtgtagggatagaggggtgtaggtaacgctgtcgatcgatcgatcgatg

SEQ ID NO: 202 - DNA, 75 bp, Artificial
  clone of aptamer
gggagaggagagaacgttctacaggagtggagctacagagagggttaggggtcgctgtcgatcgatcgatcgatg

SEQ ID NO: 203 - DNA, 75 bp, Artificial
  clone of aptamer
gggagaggagagaacgttctacggatgttgggagtgatagaaggaaggggagcgctgtcgatcgatcgatcgatg

SEQ ID NO: 204 - DNA, 75 bp, Artificial; identical to SEQ ID NO: 190
  clone of aptamer

SEQ ID NO: 205 - DNA, 75 bp, Artificial; identical to SEQ ID NO: 190
  clone of aptamer

SEQ ID NO: 206 - DNA, 75 bp, Artificial; identical to SEQ ID NO: 190
  clone of aptamer

SEQ ID NO: 207 - DNA, 76 bp, Artificial
  clone of aptamer
gggagaggagagaacgttctacttggggtggaaggagtaagggaggtgctgatcgctgtcgatcgatcgatcgatg

SEQ ID NO: 208 - DNA, 75 bp, Artificial
  clone of aptamer
gggagaggagagaacgttctacgtattaggggggaaggggaggaatagatcacgctgtcgatcgatcgatcgatg

SEQ ID NO: 209 - DNA, 76 bp, Artificial
  clone of aptamer
gggagaggagagaacgttctacagggagagagtgttgagtgaagaggaggagtcgctgtcgatcgatcgatcgatg

SEQ ID NO: 210 - DNA, 75 bp, Artificial
  clone of aptamer
gggagaggagagaacgttctacattgtgctcctggggcccagtggggagccacgctgtcgatcgatcgatcgatg

SEQ ID NO: 211 - DNA, 75 bp, Artificial
  clone of aptamer
gggagaggagagaacgttctacgagcagccctggggcccggagggggatggtcgctgtcgatcgatcgatcgatg

SEQ ID NO: 212 - DNA, 75 bp, Artificial; identical to SEQ ID NO: 190
  clone of aptamer

SEQ ID NO: 213 - DNA, 75 bp, Artificial
  clone of aptamer
gggagaggagagaacgttctaccaacggcatcctgggccccacaggggatgtcgctgtcgatcgatcgatcgatg

SEQ ID NO: 214 - DNA, 74 bp, Artificial
  clone of aptamer
gggagaggagagaacgttctacgagtggatagggaagaaggggagtagtcacgctgtcgatcgatcgatcgatg

SEQ ID NO: 215 - DNA, 75 bp, Artificial
  clone of aptamer
gggagaggagagaacgttctacccgcagcatagcctggggacccatggggggcgctgtcgatcgatcgatcgatg

SEQ ID NO: 216 - DNA, 76 bp, Artificial
  clone of aptamer
gggagaggagagaacgttctacggtcgcgtgtgggggacggatgggtattggtcgctgtcnatcgatcgatcnatg

SEQ ID NO: 217 - DNA, 75 bp, Artificial; identical to SEQ ID NO: 215
  clone of aptamer

SEQ ID NO: 218 - DNA, 75 bp, Artificial; identical to SEQ ID NO: 215
  clone of aptamer

SEQ ID NO: 219 - DNA, 75 bp, Artificial
  clone of aptamer
gggagaggagagaacgttctacggggttacgtcgcacgatacatgcattcatcgctgtcgatcgatcgatcgatg

SEQ ID NO: 220 - DNA, 75 bp, Artificial
  clone of aptamer
gggagaggagagaacgttctactagcgaggaggggttttctatttttgcgatcgctgtcgatcgatcgatcgatg

SEQ ID NO: 221 - DNA, 75 bp, Artificial
  clone of aptamer
gggagaggagagaacgttctacgtgtgatggggtgagaggatgagttagtgacgctgtcgatcgatcgatcgatg

SEQ ID NO: 222 - DNA, 74 bp, Artificial
  clone of aptamer
gggagaggagagaacgttctacaatgggagggtaatagtgatgaggagaggcgctgtcgatcgatcgatcgatg

SEQ ID NO: 223 - DNA, 75 bp, Artificial
  clone of aptamer
gggagaggagagaacgttctacgggtcgtgagataatggctcccgtattcagcgctgtcgatcgatcgatcgatg

SEQ ID NO: 224 - DNA, 75 bp, Artificial; identical to SEQ ID NO: 223
  clone of aptamer

SEQ ID NO: 225 - DNA, 75 bp, Artificial
  clone of aptamer
gggagaggagagaacgttctacaggtagcgtgagggggtgttaatagaggggcgctgtcgatcgatcgatcgatg

SEQ ID NO: 226 - DNA, 75 bp, Artificial
  clone of aptamer
gggagaggagagaacgttctacgataggatgggtgggacaggagagggagtgcgctgtcgatcgatcgatcgatg

SEQ ID NO: 227 - DNA, 75 bp, Artificial
  clone of aptamer
gggagaggagagaacgttctaccagtgagggcagtgtcagattgagaggaggcgctgtcgatcgatcgatcgatg

SEQ ID NO: 228 - DNA, 75 bp, Artificial
  clone of aptamer
gggagaggagagaacgttctaccttgcctaacaggaggtggagtattggacccgctgtcgatcgatcgatcgatg

SEQ ID NO: 229 - DNA, 75 bp, Artificial; identical to SEQ ID NO: 228
  clone of aptamer

SEQ ID NO: 230 - DNA, 73 bp, Artificial
  clone of aptamer
gggagaggagagaacgttctacgtcgtgagtaatggctcgtagatgaggtcgctgtcgatcgatcgatcgatg

SEQ ID NO: 231 - DNA, 74 bp, Artificial
  clone of aptamer
gggagaggagagaacgttctacgggattaagaggggagaggagcagttgagcgctgtcgatcgatcgatcgatg

SEQ ID NO: 232 - DNA, 75 bp, Artificial
  clone of aptamer
gggagaggagagaacgttctactccggttggggtatcaggtctacggactgacgctgtcgatcgatcgatcgatg

SEQ ID NO: 233 - DNA, 75 bp, Artificial; identical to SEQ ID NO: 223
  clone of aptamer

SEQ ID NO: 234 - DNA, 75 bp, Artificial; identical to SEQ ID NO: 223
  clone of aptamer

SEQ ID NO: 235 - DNA, 75 bp, Artificial
  clone of aptamer
gggagaggagagaacgttctacatgacaagagggggttgtgtgggatggcagcgctgtcgatcgatcgatcgatg

SEQ ID NO: 236 - DNA, 76 bp, Artificial
  clone of aptamer
gggagaggagagaacgttctacacagggaggggagcggagaggagagagggtacgctgtcgatcgatcgatcgatg

SEQ ID NO: 237 - DNA, 75 bp, Artificial; identical to SEQ ID NO: 223
  clone of aptamer

SEQ ID NO: 238 - DNA, 73 bp, Artificial; identical to SEQ ID NO: 230
  clone of aptamer

SEQ ID NO: 239 - DNA, 75 bp, Artificial; identical to SEQ ID NO: 223
  clone of aptamer

SEQ ID NO: 240 - DNA, 75 bp, Artificial; identical to SEQ ID NO: 223
  clone of aptamer

SEQ ID NO: 241 - DNA, 75 bp, Artificial; identical to SEQ ID NO: 228
  clone of aptamer

SEQ ID NO: 242 - DNA, 75 bp, Artificial
  clone of aptamer
gggagaggagagaacgttctacggctatgcgtcgtgagtcaatggcccgcatcgctgtcgatcgatcgatcgatg

SEQ ID NO: 243 - DNA, 75 bp, Artificial
  clone of aptamer
gggagaggagagaacgttctacgggtcgtgagatagtggctcccgtattcagcgctgtcgatcgatcgatcgatg

SEQ ID NO: 244 - DNA, 75 bp, Artificial; identical to SEQ ID NO: 223
  clone of aptamer

SEQ ID NO: 245 - DNA, 75 bp, Artificial; identical to SEQ ID NO: 223
  clone of aptamer

SEQ ID NO: 246 - DNA, 75 bp, Artificial
  clone of aptamer
gggagaggagagaacgttctaccttgtctaacaggaggtggagtattggacccgctgtcgatcgatcgatcgatg

SEQ ID NO: 247 - DNA, 75 bp, Artificial
  clone of aptamer
gggagaggagagaacgttctacgactttgagggtggtgagagtggaagagagcgctgtcgatcgatcgatcgatg

SEQ ID NO: 248 - DNA, 75 bp, Artificial
  clone of aptamer
gggagaggagagaacgttctacggtagggtatgaccagggaggtattggaggcgctgtcgatcgatcgatcgatg

SEQ ID NO: 249 - DNA, 75 bp, Artificial; identical to SEQ ID NO: 223
  clone of aptamer

SEQ ID NO: 250 - DNA, 75 bp, Artificial; identical to SEQ ID NO: 223
  clone of aptamer

SEQ ID NO: 251 - DNA, 72 bp, Artificial
  clone of aptamer
gggagaggagagaacgttctacgttatgcatgtggagagtgagagagggcgctgtcgatcgatcgatcgatg

SEQ ID NO: 252 - DNA, 75 bp, Artificial
  clone of aptamer
gggagaggagagaacgttctaccatgtctgcgggaggtgagtagtgatcctgcgctgtcgatcgatcgatcgatg

SEQ ID NO: 253 - DNA, 75 bp, Artificial
  clone of aptamer
gggagaggagagaacgttctacagagtgggagggatgtgtgacacaggtaggcgctgtcgatcgatcgatcgatg

SEQ ID NO: 254 - DNA, 73 bp, Artificial
  clone of aptamer
gggagaggagagaacgttctacgctccatgacagtgaggtgagtagtgatcgctgtcgatcgatcgatcgatg

SEQ ID NO: 255 - DNA, 74 bp, Artificial
  clone of aptamer
gggagaggagagaacgttctcgatgctgacagggtgtgttcagtaatggctcgctgtcgatcgatcgatcgatg

SEQ ID NO: 256 - DNA, 75 bp, Artificial
  clone of aptamer
gggagaggagagaacgttctaccagcaaacagggtcaggtgagtagtgatgacgctgtcgatcgatcgatcgatg

SEQ ID NO: 257 - DNA, 75 bp, Artificial
  clone of aptamer
gggagaggagagaacgttctacgacaagccgggggtgttcagtagtggcaaccgctgtcgatcgatcgatcgatg

SEQ ID NO: 258 - DNA, 75 bp, Artificial
  clone of aptamer
gggagaggagagaacgttctacatatggcgctggaggtgagtaatgatcgtgcgctgtcgatcgatcgatcgatg

SEQ ID NO: 259 - DNA, 75 bp, Artificial
  clone of aptamer
gggagaggagagaacgttctacggggcgatagcgttcagtagtggcgccggtcgctgtcgatcgatcgatcgatg

SEQ ID NO: 260 - DNA, 74 bp, Artificial
  clone of aptamer
gggagaggagagaacgttctacatagcggactgggtgcatggagcggcgcacgctgtcgatcgatcgatcgatg

SEQ ID NO: 261 - DNA, 74 bp, Artificial
  clone of aptamer
gggagaggagagaacgttctacgggtcaacaggggcgttcagtagtggcggcgctgtcgatcgatcgatcgatg

SEQ ID NO: 262 - DNA, 75 bp, Artificial
  clone of aptamer
gggagaggagagaacgttctacgcatgcgagctgaggtgagtagtgatcagtcgctgtcgatcgatcgatcgatg

SEQ ID NO: 263 - DNA, 74 bp, Artificial
  clone of aptamer
gggagaggagagaacgttctacatgcgacaggggagtgttcagtagtggcacgctgtcgatcgatcgatcgatg

SEQ ID NO: 264 - DNA, 75 bp, Artificial
  clone of aptamer
gggagaggagagaacgttctaccccatcgtatggagtgcggaacggggcatacgctgtcgatcgatcgatcgatg

SEQ ID NO: 265 - DNA, 72 bp, Artificial
  clone of aptamer
gggagaggagagaacgttctacagtgaggcgggagcgtttcagtaatggcgctgtcgatcgatcgatcgatg

SEQ ID NO: 266 - DNA, 74 bp, Artificial
  clone of aptamer
gggagaggagagaacgttctacacagcgtcgggtgttcagtaatggcgcagcgctgtcgatcgatcgatcgatg

SEQ ID NO: 267 - DNA, 75 bp, Artificial
  clone of aptamer
gggagaggagagaacgttctacggtgttcagtagtggcacaggaggaagggatgctgtcgatcgatcgatcgatg

SEQ ID NO: 268 - DNA, 75 bp, Artificial
  clone of aptamer
gggagaggagagaacgttctacagttcaggcgttaggcatgggtgtcgctttcgctgtcgatcgatcgatcgatg

SEQ ID NO: 269 - DNA, 75 bp, Artificial
  clone of aptamer
gggagaggagagaacgttctacatgcgacatgcgagtgttcagtagcggcagcgctgtcgatcgatcgatcgatg

SEQ ID NO: 270 - DNA, 75 bp, Artificial
  clone of aptamer
gggagaggagagaacgttctacctatggcgttacagcgaggtgagtagtgatcgctgtcgatcgatcgatcgatg

SEQ ID NO: 271 - DNA, 75 bp, Artificial
  clone of aptamer
gggagaggagagaacgttctaccagccgatccagccaggcgttcagtagtggcgctgtcgatcgatcgatcgatg

SEQ ID NO: 272 - DNA, 74 bp, Artificial
  clone of aptamer
gggagaggagagaacgttctacggcacaggcacggcgaggtgagtaatgatcgctgtcgatcgatcgatcgatg

SEQ ID NO: 273 - DNA, 73 bp, Artificial
  clone of aptamer
gggagaggagagaacgttctactgtggacagcgggagtgcggaacggggtcgctgtcgatcgatcgatcgatg

SEQ ID NO: 274 - DNA, 75 bp, Artificial
  clone of aptamer
gggagaggagagaacgttctactgatgctgcgagtgcatggggcaggcgcttcgctgtcgatcgatcgatcgatg

SEQ ID NO: 275 - DNA, 75 bp, Artificial
  clone of aptamer
gggagaggagagaacgttctacggtacaatgggaatgacagtgatgggtagccgctgtcgatcgatcgatcgatg

SEQ ID NO: 276 - DNA, 73 bp, Artificial
  clone of aptamer
gggagaggagagaacgttctacatggacagcgaagcatgggggaggcgcacgctgtcgatcgatcgatcgatg

SEQ ID NO: 277 - DNA, 75 bp, Artificial
  clone of aptamer
gggagaggagagaacgttctactgggagcgacagtgagcatggggtaggcgccgctgtcgatcgatcgatcgatg

SEQ ID NO: 278 - DNA, 74 bp, Artificial
  clone of aptamer
gggagaggagagaacgttctaccggcgagcaggtgttcagtagtggctttgcgctgtcgatcgatcgatcgatg

SEQ ID NO: 279 - DNA, 75 bp, Artificial
  clone of aptamer
gggagaggagagaacgttctacgatcagtgagggagtgcagtagtggctcgtcgctgtcgatcgatcgatcgatg

SEQ ID NO: 280 - DNA, 81 bp, Artificial
  clone of aptamer
gggagaggagagaacgttctacaaatgagagcaggccgaaaaggagtcgctcgctgtcgatcgatcgatcgatgaagggcg

SEQ ID NO: 281 - DNA, 82 bp, Artificial
  clone of aptamer
gggagaggagagaacgttctacaaaggatcaatctttcggcgtatgtgtgagcgctgtcgatcgatcgatcgatgaagggcg

SEQ ID NO: 282 - DNA, 82 bp, Artificial
  clone of aptamer
gggagaggagagaacgttctacggtaaagcaggctgactgaaaggttgaagtcgctgtcgatcgatcgatcgatgaagggcg

SEQ ID NO: 283 - DNA, 81 bp, Artificial
  clone of aptamer
gggagaggagagaacgttctacaggttaaaagcaggctcaggaatggaagtcgctgtcgatcgatcgatcgatgaagggcg

SEQ ID NO: 284 - DNA, 82 bp, Artificial
  clone of aptamer
gggagaggagagaacgttctacaacaaagcaggctcatagtaatatggaagtcgctgtcgatcgatcgatcgatgaagggcg

SEQ ID NO: 285 - DNA, 81 bp, Artificial
  clone of aptamer
gggagaggagagaacgttctacaaaagagagcaggccgaaaaggagtcgctcgctgtcgatcgatcgatcgatgaagggcg

SEQ ID NO: 286 - DNA, 82 bp, Artificial
  clone of aptamer
gggagaggagagaacgttctacaaaaggcaggctcaggggatcactggaagtcgctgtcgatcgatcgatcgatgaagggcg

SEQ ID NO: 287 - DNA, 82 bp, Artificial
  clone of aptamer
gggagaggagagaacgttctacaaaaagcaggccgtatggatataagggagtcgctgtcgatcgatcgatcgatgaagggcg

SEQ ID NO: 288 - DNA, 82 bp, Artificial
  clone of aptamer
gggagaggagagaacgttctacaaaagtgcaggctgcagacatatgcgaagtcgctgtcgatcgatcgatcgatgaagggcg

SEQ ID NO: 289 - DNA, 81 bp, Artificial
  clone of aptamer
gggagaggagagaacgttctacaaaggagagcaggccgaaaaggagtcgctcgctgtcgatcgatcgatcgatgaagggcg

SEQ ID NO: 290 - DNA, 83 bp, Artificial
  clone of aptamer
gggagaggagagaacgttctacaagatataattaaggataagtgcaaaggagacgctgtcgatcgatcgatcgatgaagggcg

SEQ ID NO: 291 - DNA, 84 bp, Artificial
  clone of aptamer
gggagaggagagaacgttctacagacaacagcnagagggaatcncanacaaagacgctgtcgatcgatcgatcgatgaagggcg

SEQ ID NO: 292 - DNA, 82 bp, Artificial
  clone of aptamer
gggagaggagagaacgttctacagattctaagcgcaggaataagtcaccagacgctgtcgatcgatcgatcgatgaagggcg

SEQ ID NO: 293 - DNA, 82 bp, Artificial
  clone of aptamer
gggagaggagagaacgttctacgaaaatgagcatggaagtgggagtacgtgccgctgtcgatcgatcgatcgatgaagggcg

SEQ ID NO: 294 - DNA, 82 bp, Artificial
  clone of aptamer
gggagaggagagaacgttctacgaaaagaggcgccggaagtgagagtaagtgcgctgtcgatcgatcgatcgatgaagggcg

SEQ ID NO: 295 - DNA, 82 bp, Artificial
  clone of aptamer
gggagaggagagaacgttctacgaagtgagtttccgaagtgagagtacgaaacgctgtcgatcgatcgatcgatgaagggcg

SEQ ID NO: 296 - DNA, 81 bp, Artificial
  clone of aptamer
gggagaggagagaacgttctacgaatgagagcaggccgaaaaggagtcgctcgctgtcgatcgatcgatcgatgaagggcg

SEQ ID NO: 297 - DNA, 82 bp, Artificial
  clone of aptamer
gggagaggagagaacgttctacgagaggcaagagagagtcgcataaaaaagacgctgtcgatcgatcgatcgatgaagggcg

SEQ ID NO: 298 - DNA, 82 bp, Artificial
  clone of aptamer
gggagaggagagaacgttctacgcaggctgtcgtagacaaacgatgaagtcgcgctgtcgatcgatcgatcgatgaagggcg

SEQ ID NO: 299 - DNA, 83 bp, Artificial
  clone of aptamer
gggagaggagagaacgttctacggaaaaagatatgaaagaaaggattaagagacgctgtcgatcgatcgatcgatgaagggcg

SEQ ID NO: 300 - DNA, 82 bp, Artificial
  clone of aptamer
gggagaggagagaacgttctacggaaggnaacaanagcactgtttgtgcaggcgctgtcgatcnatcnatcnatgaagggcg

SEQ ID NO: 301 - DNA, 82 bp, Artificial
  clone of aptamer
gggagaggagagaacgttctacggagcatanggcntgaaactgaganagtaacgctgtcgatcgatcgatcgatgaagggcg

SEQ ID NO: 302 - DNA, 83 bp, Artificial
  clone of aptamer
gggagaggagagaacgttctacgaaaaaggatatgagagaaaggattaagagacgctgtcgatcgatcgatcgatgaagggcg

SEQ ID NO: 303 - DNA, 82 bp, Artificial
  clone of aptamer
gggagaggagagaacgttctacatacataggcgccgcgaatgggaaagaaagcgctgtcgatcgatcgatcgatgaagggcg

SEQ ID NO: 304 - DNA, 82 bp, Artificial
  clone of aptamer
gggagaggagagaacgttctactcatgaagccatggttgtaattctgtttggcgctgtcgatcgatcgatcgatgaagggcg

SEQ ID NO: 305 - DNA, 80 bp, Artificial
  clone of aptamer
gggagaggagagaacgttctactaatgcaggctcagttactactggaagtcgctgtcgatcgatcgatcgatgaagggcg

SEQ ID NO: 306 - DNA, 81 bp, Artificial
  clone of aptamer
gggagaggagagaacgttctactttcataggcgggattatggaggagtattcgctgtcgatcgatcgatcgatgaagggcg

SEQ ID NO: 307 - DNA, 82 bp, Artificial
  clone of aptamer
aggagaggagagaacgttctactagaagcaggctcgaatacaattcggaagtcgctgtcgatcgatcgatcgatgaagggcg

SEQ ID NO: 308 - DNA, 82 bp, Artificial
  clone of aptamer
gggagaggagagaacgttctacttagcgatgtcggaagagagagtacgaggacgctgtcgatcgatcgatcgatgaagggcg

SEQ ID NO: 309 - DNA, 82 bp, Artificial
  clone of aptamer
gggagaggagagaacgttctacttgcgaagaccgtggaagaggagtactggtcgctgtcgatcgatcgatcgatgaagggcg

SEQ ID NO: 310 - DNA, 82 bp, Artificial
  clone of aptamer
gggagaggagagaacgttctacttttggtgaaggtgtaagagtggcactacacgctgtcgatcgatcgatcgatgaagggcg

SEQ ID NO: 311 - DNA, 82 bp, Artificial
  clone of aptamer
gggagaggagagaacgttctaccatcagttgtggcgattatgtgggagtatgcgctgtcgatcgatcgatcgatgaagggcg

SEQ ID NO: 312 - DNA, 83 bp, Artificial
  clone of aptamer
gggagaggagagaacgttctacanaanaacatgcgattaaagatcatgaacagcgctgtcgatcgatcgatcgatgaagggcg

SEQ ID NO: 313 - DNA, 82 bp, Artificial
  clone of aptamer
gggagaggagagaacgttctacataagcaggctccgatagtattcgggaagtcgctgtcgatcgatcgatcgatgaagggcg

SEQ ID NO: 314 - DNA, 82 bp, Artificial
  clone of aptamer
gggagaggagagaacgttctactttcggaatgcgatgggggtgattcgtggtcgctgtcgatcgatcgatcgatgaagggcg

SEQ ID NO: 315 - DNA, 80 bp, Artificial
  clone of aptamer
gggagaggagagaacgttctacctgttgaggctaagtggatgattgagggcgctgtcgatcgatcgatcgatgaagggcg

SEQ ID NO: 316 - DNA, 81 bp, Artificial
  clone of aptamer
gggagaggagagaacgttctacctgggtcggtgcgattggagatgtcgttgcgctgtcgatcgatcgatcgatgaagggcg

SEQ ID NO: 317 - DNA, 82 bp, Artificial
  clone of aptamer
gggagaggagagaacgttctacctgatgtcaggttgtttggagattatctgacnctgtcnatcgatcgatcgatgaagggcg

SEQ ID NO: 318 - DNA, 82 bp, Artificial
  clone of aptamer
gggagaggagagaacgttctacctcgcgcgacgagcgaatttccggatgcggcgctgtcgatcgatcgatcgatgaagggcg

SEQ ID NO: 319 - DNA, 82 bp, Artificial
  clone of aptamer
gggagaggagagaacgttctaccatgaatgattgcgatcgttgttcgtgtggcgctgtcgatcgatcgatcgatgaagggcg

SEQ ID NO: 320 - DNA, 83 bp, Artificial
  clone of aptamer
gggagaggagagaacgttctactccgaccacgcctgggtgattcctacnacgacgctgtcgatcgatcgatcgatgaagggcg

SEQ ID NO: 321 - DNA, 82 bp, Artificial
  clone of aptamer
gggagaggagagaacgttctactacttttggggattcactccgcgctgatgtcgctgtcgatcgatcgatcgatgaagggcg

SEQ ID NO: 322 - DNA, 82 bp, Artificial
  clone of aptamer
gggagaggaganaacgttctantagtgcttgcgagatagtgtaggattatactgctgtcgatcgatcgatcgatgaagggcg

SEQ ID NO: 323 - DNA, 82 bp, Artificial
  clone of aptamer
gggagaggagagaacgttctactagtgtccttctccacgtggttgtaattgtcgctgtcgatcgatcgatcgatgaagggcg

SEQ ID NO: 324 - DNA, 82 bp, Artificial
  clone of aptamer
gggagaggagagaacgttctactattgtggcgcttgttggactaactgactacgctgtcgatcgatcgatcgatgaagggcg

SEQ ID NO: 325 - DNA, 82 bp, Artificial
  clone of aptamer
gggagaggagagaacgtcctacttcgattgtgatcttgtggcggcctgtgagcgctgtcgatcgatcgatcgatgaagggcg

SEQ ID NO: 326 - DNA, 82 bp, Artificial
  clone of aptamer
gggagaggagagaacgttctacttggcgatgtcggaagagagagtacgagggcgctgtcgatcgatcgatcgatgaagggcg

SEQ ID NO: 327 - DNA, 82 bp, Artificial
  clone of aptamer
gggagaggagagaacgttctacttgaanctgcgtgaattganagtaacgaagcgctgtcaatcgatcnatcaatnaagggcg

SEQ ID NO: 328 - DNA, 82 bp, Artificial
  clone of aptamer
gggagaggagagaacgttctactcgagaggacatgtggatccggttcgcgtgcgctgtcgatcgatcgatcgatgaagggcg

SEQ ID NO: 329 - DNA, 82 bp, Artificial
  clone of aptamer
gggagaggagagaacgttctactgtgatgcggtttgcgtcgaccggattcgtcgctgtcgatcgatcgatcgatgaagggcg

SEQ ID NO: 330 - DNA, 81 bp, Artificial
  clone of aptamer
gggagaggagagaacgttctactgtgtgattgggcgcatgtcgaggcgacacgctgtcgatcgatcgatcgatgaagggcg

SEQ ID NO: 331 - DNA, 81 bp, Artificial
  clone of aptamer
gggagaggagagaacgttctactgattaagatgcgctggtagagcggtgggcgctgtcgatcgatcgatcgatgaagggcg

SEQ ID NO: 332 - DNA, 82 bp, Artificial
  clone of aptamer
gggagaggagagaacgttctactggttaatttgcatgcgcgantaacntgntcgctgtcgatcgatcgatcgatgaagggcg

SEQ ID NO: 333 - DNA, 81 bp, Artificial
  clone of aptamer
gggagaggagagaacgttctactgggaagcggtaacttggattgaccgatccgctgtcgatcgatcgatcgatgaagggcg

SEQ ID NO: 334 - DNA, 82 bp, Artificial
  clone of aptamer
gggagaggagagaacgttctactgttacggagatgatgggtttggctgttggcgctgtcgatcgatcgatcgatgaagggcg

SEQ ID NO: 335 - DNA, 81 bp, Artificial
  clone of aptamer
gggagaggagagaacgttctacttgtggactgagatacgattcggagctggcgctgtcgatcgatcgatcgatgaagggcg

SEQ ID NO: 336 - DNA, 82 bp, Artificial
  clone of aptamer
gggagaggagagaacgttctacttgtgagtttccttgggccttgagcgtgggcgctgtcgatcgatcgatcgatgaagggcg

SEQ ID NO: 337 - DNA, 82 bp, Artificial
  clone of aptamer
gggagaggagagaacgttctacaggtgatgtgagccgattgtgaagttttgtcgctgtcgatcgatcgatcgatgaagggcg

SEQ ID NO: 338 - DNA, 82 bp, Artificial
  clone of aptamer
gggagaggagagaacgttctacagcggatgtttgggggtgtgtgttggttgtcgctgtcgatcgatcgatcgatgaagggcg

SEQ ID NO: 339 - DNA, 82 bp, Artificial
  clone of aptamer
gggagaggagagaacgttctacatgcggtggtggtcttcgatgggtggaagtcgctgtcgatcgatcgatcgatgaagggcg

SEQ ID NO: 340 - DNA, 82 bp, Artificial
  clone of aptamer
gggagaggagagaacgttctacattggaggggcgcatgtggtctgtttgatgcgctgtcgatcgatcgatcgatgaagggcg

SEQ ID NO: 341 - DNA, 82 bp, Artificial
  clone of aptamer
gggagaggagagaacgttctacgtgtttcgcggatttgaagaggagtaaaatcgctgtcgatcgatcgatcgatgaagggcg

SEQ ID NO: 342 - DNA, 82 bp, Artificial
  clone of aptamer
gggagaggagagaacgttctacgtgtgcgtgttcgggaagggagagtgccgaggctgtcgatcgatcgatcgatgaagggcg

SEQ ID NO: 343 - DNA, 82 bp, Artificial
  clone of aptamer
gggagaggagagaacgttctacgtgtgtggtgtgcgatgcttggctgtttgtcgctgtcgatcgatcgatcgatgaagggcg

SEQ ID NO: 344 - DNA, 82 bp, Artificial
  clone of aptamer
gggagaggagagaacgttctacggtttgtgtggcttggatctgaagactaagcgctgtcgatcgatcgatcgatgaagggcg

SEQ ID NO: 345 - DNA, 82 bp, Artificial
  clone of aptamer
gggagaggagagaacgttctacggttctgggcttgtgtgtgaggattgacggcgctgtcgatcgatcgatcgatgaagggcg

SEQ ID NO: 346 - DNA, 74 bp, Artificial
  clone of aptamer
gggagaggagagaacgttctacgatgatgaaggcgaaaagacgaggctgtcgatcgatcgatcgatgaagggcg

SEQ ID NO: 347 - DNA, 82 bp, Artificial
  clone of aptamer
gggagaggagagaacgttctacgagtgctgatgcgtgtcctgggatggaattcgctgtcgatcgatcgatcgatgaagggcg

SEQ ID NO: 348 - DNA, 82 bp, Artificial
  clone of aptamer
gggagaggagagaacgttctacgcgtttatagcgatcgatgatgatataggccgctgtcgatcgatcgatcgatgaagggcg

SEQ ID NO: 349 - DNA, 82 bp, Artificial
  clone of aptamer
gggagaggagagaacgttctacgcgttcaaatgggatagaattggctgcgggcgctgtcgatcgatcgatcgatgaagggcg

SEQ ID NO: 350 - DNA, 79 bp, Artificial
  clone of aptamer
gggagaggagagaacgttctacgaaattgtgcgtcagtgtgaggcggtttgctgtcgatcgatcgatcgatgaagggcg

SEQ ID NO: 351 - DNA, 82 bp, Artificial
  clone of aptamer
gggagaggagagaacgttctacggtcgaaatgagggtctggagttccgacgacgctgtcgatcgatcgatcgatgaagggcg

SEQ ID NO: 352 - DNA, 82 bp, Artificial
  clone of aptamer
gggagaggagagaacgttctacgaatttggtaatctgggtgacttaggatgtcgctgtcgatcgatcgatcgatgaagggcg

SEQ ID NO: 353 - DNA, 81 bp, Artificial
  clone of aptamer
gggagaggagagaacgttctacgattttttgtgccgaagtaagagtacgcgcgctgtcgatcgatcgatcgatgaagggcg

SEQ ID NO: 354 - DNA, 82 bp, Artificial
  clone of aptamer
aggagaggagagaacgttctacggagtgtgcgcggatgaaaacagaagttgtcgctgtcnatcgatcnatcaatgaagggcg

SEQ ID NO: 355 - DNA, 82 bp, Artificial
  clone of aptamer
gggagaggagagaacgttctacgatctgggcgagccagtctgactgaggaagcgctgtcgatcgatcgatcgatgaagggcg

SEQ ID NO: 356 - DNA, 82 bp, Artificial
  clone of aptamer
gggagaggagagaacgttctacgaagaagatatgagagaaaggattaagagacgctgtcgatcgatcgatcgatgaagggcg

SEQ ID NO: 357 - DNA, 82 bp, Artificial
  clone of aptamer
gggagaggagagaacgttctacgaaaaagatatgagagaaaggattaagagacgctgtcgatcgatcgatcgatgaagggcg

SEQ ID NO: 358 - DNA, 82 bp, Artificial
  clone of aptamer
gggagaggagagaacgttctacgaaaaagatatgagagaaaggattaagaggcgctgtcgatcgatcgatcgatgaagggcg

SEQ ID NO: 359 - DNA, 82 bp, Artificial
  clone of aptamer
gggagaggagagaacgttctacgaaaaagacatgagagaaaggattaagagacgctgtcgatcgatcgatcgatgaagggcg

SEQ ID NO: 360 - DNA, 83 bp, Artificial
  clone of aptamer
gggagaggagagaacgttctacnaaaaagtatatgagagaaaggattaanagacgctgtcgatcgatcgatcgatgaagggcg

SEQ ID NO: 361 - DNA, 83 bp, Artificial
  clone of aptamer
gggagaggagagaacgttctacgaaaaagatatgagagaaaaggattgagagatgctgtcgatcgatcgatcgatgaagggcg

SEQ ID NO: 362 - DNA, 83 bp, Artificial
  clone of aptamer
gggagaggagagcacgttctacgaaaaagatatggagagaaaggattaagagacgctgtcgatcgatcgatcgatgaagggcg

SEQ ID NO: 363 - DNA, 84 bp, Artificial
  clone of aptamer
gggagaggagagaacgttctacgaaaaagatatgagagaaaggattaaaagagacgctgtcgatcgatcgatcgatgaagggcg

SEQ ID NO: 364 - DNA, 85 bp, Artificial
  clone of aptamer
gggagaggagagaacgttctacgaanaagatacatagtagaaaggattaataagacgctgtcgatcgatcgatcgatgaagggcg

SEQ ID NO: 365 - DNA, 82 bp, Artificial
  clone of aptamer
gggagaggagagaacgttctacaggcgtgttggtagggtacgacgaggcatgcgctgtcgatcgatcgatcgatgaagggcg

SEQ ID NO: 366 - DNA, 82 bp, Artificial
  clone of aptamer
gggagaggagagaacgttctacgcaaaaatgtgatgcgaggtaatggaacgccgctgtcgatcgatcgatcgatgaagggcg

SEQ ID NO: 367 - DNA, 82 bp, Artificial
  clone of aptamer
gggagaggagagaacgttctacggacctcagcgataggggttgaaaccgacacgctgtcgatcgatcgatcgatgaagggcg

SEQ ID NO: 368 - DNA, 82 bp, Artificial
  clone of aptamer
gggagaggagagaacgttctacatggtcggatgctggggagtaggcaaggttcgctgtcgatcgatcgatcgatgaagggcg

SEQ ID NO: 369 - DNA, 82 bp, Artificial
  clone of aptamer
gggagaggagagaacgttctacgtatcggcgagcgaagcatccgggagcgttcgctgtcgatcgatcgatcgatgaagggcg

SEQ ID NO: 370 - DNA, 82 bp, Artificial
  clone of aptamer
gggagaggagagaacgttctacgtattggcgcgcgaagcatccgggagcgttcgctgtcgatcgatcgatcgatgaagggcg

SEQ ID NO: 371 - DNA, 82 bp, Artificial
  clone of aptamer
gggagaggagagaacgttctacttatacctgacggccggaggcgcataggtgcgctgtcgatcgatcgatcgatgaagggcg

SEQ ID NO: 372 - DNA, 82 bp, Artificial; identical to SEQ ID NO: 368
  clone of aptamer

SEQ ID NO: 373 - DNA, 82 bp, Artificial
  clone of aptamer
gggagaggagagaacgttctacacgagagtactgaggcgcttggtacagagtcgctgtcgatcgatcgatcgatgaagggcg

SEQ ID NO: 374 - DNA, 82 bp, Artificial
  clone of aptamer
gggagaggagagaacgttctacagaaggtagaaaaaggatagctgtgagaagcgctgtcgatcgatcgatcgatgaagggcg

SEQ ID NO: 375 - DNA, 82 bp, Artificial
  clone of aptamer
gggagaggagagaacgttctactgagggataatacgggtgggattgtcttcccgctgtcgatcgatcgatcgatgaagggcg

SEQ ID NO: 376 - DNA, 84 bp, Artificial
  clone of aptamer
gggagaggagagaacgttctacattgagcgttgaagttggggaagctccgaggccgctgtcgatcgatcgatcgatgaagggcg

SEQ ID NO: 377 - DNA, 82 bp, Artificial
  clone of aptamer
gggagaggagagaacgttctacgcggagatatacagcgaggtaatggaacgccgctgtcgatcgatcgatcgatgaagggcg

SEQ ID NO: 378 - DNA, 82 bp, Artificial
  clone of aptamer
gggagaggagagaacgttctacgaagacagcccaatagcggcacggaacttgcgctgtcgatcgatcgatcgatgaagggcg

SEQ ID NO: 379 - DNA, 84 bp, Artificial
  clone of aptamer
gggagaggagagaacgttctaccggttgagggctcgcgtggaagggccaacacgcgctgtcgatcgatcgatcgatgaagggcg

SEQ ID NO: 380 - DNA, 82 bp, Artificial
  clone of aptamer
gggagaggagagaacgttctacatatcaatagactcttgacgtttgggtttgcgctgtcgatcgatcgatcgatgaagggcg

SEQ ID NO: 381 - DNA, 79 bp, Artificial
  clone of aptamer
gggagaggagagaacgttctacagtgaaggaaaagtaagtgaaggtgtgcgctgtcgatcgatcgatcgatgaagggcg

SEQ ID NO: 382 - DNA, 82 bp, Artificial
  clone of aptamer
gggagaggagagaacgttctacggatgaaatgagtgtctgcgataggttaagcgctgtcgatcgatcgatcgatgaagggcg

SEQ ID NO: 383 - DNA, 83 bp, Artificial
  clone of aptamer
gggagaggagagaacgttctacggaaggaaatgtgtgtctgcgataggttaagcgctgtcgatcgatcgatcgatgaagggcg

SEQ ID NO: 384 - DNA, 82 bp, Artificial
  clone of aptamer
gggagaggagagaacgttctacatccttgcgtatgatcggcatcgtaagacacgctgtcgatcgatcgatcgatgaagggcg

SEQ ID NO: 385 - DNA, 82 bp, Artificial; identical to SEQ ID NO: 384
  clone of aptamer

SEQ ID NO: 386 - DNA, 77 bp, Artificial
  clone of aptamer
gggagaggagagaacgttctacgatcgaagtcgtgacagaaaccactcgctgtcgatcgatcgatcgatgaagggcg

SEQ ID NO: 387 - DNA, 77 bp, Artificial; identical to SEQ ID NO: 386
  clone of aptamer

SEQ ID NO: 388 - DNA, 82 bp, Artificial
  clone of aptamer
gggagaggagagaacgttctacggaaaaggttggcgaaacgaagaagaatttcgctgtcgatcgatcgatcgatgaagggcg

SEQ ID NO: 389 - DNA, 82 bp, Artificial
  clone of aptamer
gggagaggagagaacgttctacggaaaaggttggcgaaacgaagaanaatttcgctgtcgatcgatcgatcgatgaagggcg

SEQ ID NO: 390 - DNA, 83 bp, Artificial
  clone of aptamer
gggagaggagagaacgttctactgggagttgcggtgttttgcggtggatttgacgctgtcgatcgatcgatcgatgaagggcg

SEQ ID NO: 391 - DNA, 83 bp, Artificial; identical to SEQ ID NO: 390
  clone of aptamer

SEQ ID NO: 392 - DNA, 82 bp, Artificial
  clone of aptamer
gggagaggagagaacgctctacaagattgtagatcaacagcgaaggcgtgggcgctgtcgatcgatcgatcgatgaagggcg

SEQ ID NO: 393 - DNA, 82 bp, Artificial; identical to SEQ ID NO: 392
  clone of aptamer

SEQ ID NO: 394 - DNA, 82 bp, Artificial
  clone of aptamer
gggagaggagagaacgttctacaaanaagatnnccancnngaganaaaggagcgctgtcgatcgatcgatcgatgaagggcg

SEQ ID NO: 395 - DNA, 82 bp, Artificial
  clone of aptamer
gggagaggagagaacgttctacaaacatcgaagatcgaactgaaaaggagggcgctgtcgatcgatcgatcgatgaagggcg

SEQ ID NO: 396 - DNA, 82 bp, Artificial
  clone of aptamer
gggagaggagagaacgttctacatgtgcatgcaaggtggggctgacacgagccgctgtcgatcgatcgatcgatgaagggcg

SEQ ID NO: 397 - DNA, 80 bp, Artificial
  clone of aptamer
gggagaggagagaacgttctacaaggagtagatcgacagaatagaaaaatcgctgtcgatcgatcgatcgatgaagggcg

SEQ ID NO: 398 - DNA, 83 bp, Artificial
  clone of aptamer
gggagaggagagaacgttctacaaaaggtaaggtcaaaaaagcgcaacgttgacgctgtcgatcgatcgatcgatgaagggcg

SEQ ID NO: 399 - DNA, 82 bp, Artificial
  clone of aptamer
gggagaggagagaacgttctacaaaaggaggcgaaataagtgagacaatgtgcgctgtcgatcgatcgatcgatgaagggcg

SEQ ID NO: 400 - DNA, 81 bp, Artificial
  clone of aptamer
gggagaggagagaacgttctacaaaaatccacaaacatagctgtaattgctcgctgtcgatcgatcgatcgatgaagggcg

SEQ ID NO: 401 - DNA, 81 bp, Artificial
  clone of aptamer
gggagaggagagacgttctacaagaacatataacattttggttgagagcaacgctgtcgatcgatcgatcgatgaagggcg

SEQ ID NO: 402 - DNA, 83 bp, Artificial
  clone of aptamer
gggagaggagagaacgttctacaagagtcnacgatttcnatcacaaatgtggctgctgtcnatcgatcgatcnatgaagggcg

SEQ ID NO: 403 - DNA, 83 bp, Artificial
  clone of aptamer
gggagaggagagaacgttctacaagcaagcaaaaaaagtatcgacagaagtggcgctgtcgatcgatcgatcgatgaagggcg

SEQ ID NO: 404 - DNA, 82 bp, Artificial
  clone of aptamer
gggagaggagagaacgttctacaagtaatatcagagcaatcggaataagagtcgctgtcgatcgatcgatcgatgaagggcg

SEQ ID NO: 405 - DNA, 82 bp, Artificial
  clone of aptamer
gggagaggagagaacgttctacagacttcgatgcgatggatttggaaatgtgcgctgtcgatcgatcgatcgatgaagggcg

SEQ ID NO: 406 - DNA, 82 bp, Artificial
  clone of aptamer
gggagaggagagaacgttctacagaaagaattacaggaacaaatacacgtgcggctgtcgatcgatcgatcgatgaagggcg

SEQ ID NO: 407 - DNA, 82 bp, Artificial
  clone of aptamer
gggagaggagagaacgttctacagaaatcaatcgaggtgatcgttatataggcgctgtcgatcgatcgatcgatgaagggcg

SEQ ID NO: 408 - DNA, 82 bp, Artificial
  clone of aptamer
gggagaggagagaacgttctacagatttggatcgacaatctcgtagaagagacgctgtcgatcgatcgatcgatgaagggcg

SEQ ID NO: 409 - DNA, 82 bp, Artificial
  clone of aptamer
gggagaggagagaacgttctacaatgcaagtttaagtgtggtgtcaaacgcacgctgtcgatcgatcgatcgatgaagggcg

SEQ ID NO: 410 - DNA, 81 bp, Artificial
  clone of aptamer
gggagaggagagaacgttctacaaataaagacacgaagatcgacggagactcgctgtcgatcgatcgatcgatgaagggcg

SEQ ID NO: 411 - DNA, 82 bp, Artificial
  clone of aptamer
gggagaggagagaacgttctacgaagatgtgtttaagaatcgaggttttcgacgctgtcgatcgatcgatcgatgaagggcg

SEQ ID NO: 412 - DNA, 81 bp, Artificial
  clone of aptamer
gggagaggagagaacgttctacgagttggcacgcatgtataggtattttggcgctgtcgatcgatcgatcgatgaagggcg

SEQ ID NO: 413 - DNA, 84 bp, Artificial
  clone of aptamer
gggagaggagagaacgttctacgaaaaaaagagatgagagaaaggattaagagacgctgtcgatcgatcgatcgatgaagggcg

SEQ ID NO: 414 - DNA, 82 bp, Artificial
  clone of aptamer
gggagaggagagaacgttctacgaaaaggaaaaaaaacgatcggcagagtcccgctgtcgatcgatcgatcgatgaagggcg

SEQ ID NO: 415 - DNA, 82 bp, Artificial
  clone of aptamer
gggagaggagagaacgttctacgattaaggaaacatttacgcgaatacatgacgctgtcgatcgatcgatcgatgaagggcg

SEQ ID NO: 416 - DNA, 81 bp, Artificial
  clone of aptamer
gggagaggagagaacgttctacgacgtttgctctgaaaataggacagaaggcgctgtcgatcgatcgatcgatgaagggcg

SEQ ID NO: 417 - DNA, 82 bp, Artificial; identical to SEQ ID NO: 411
  clone of aptamer

SEQ ID NO: 418 - DNA, 82 bp, Artificial
  clone of aptamer
gggagaggagagaacgttctaccgagatcgaaaggtaagagaaaattcatggcgctgtcgatcgatcgatcgatgaagggcg

SEQ ID NO: 419 - DNA, 82 bp, Artificial
  clone of aptamer
gggagaggagagaacgttctactaagattcgtcgttcagacagagaaagcgacgctgtcgatcgatcgatcgatgaagggcg

SEQ ID NO: 420 - DNA, 84 bp, Artificial
  clone of aptamer
gggagaggagagaacgttctaccttggcgacgatctgtgacctgaatttttgtccgctgtcgatcgatcgatcgatgaagggcg

SEQ ID NO: 421 - DNA, 84 bp, Artificial; identical to SEQ ID NO: 420
  clone of aptamer

SEQ ID NO: 422 - DNA, 83 bp, Artificial
  clone of aptamer
gggagaggagagaacgttctaccttggtctcagcagcttttaacaaagtatcccgctgtcgatcgatcgatcgatgaagggcg

SEQ ID NO: 423 - DNA, 83 bp, Artificial; identical to SEQ ID NO: 422
  clone of aptamer

SEQ ID NO: 424 - DNA, 81 bp, Artificial
  clone of aptamer
gggagaggagagaacgttctaccgctattttgttcattgaaggacttgtcacgctgtcgatcgatcgatcgatgaagggcg

SEQ ID NO: 425 - DNA, 81 bp, Artificial; identical to SEQ ID NO: 424
  clone of aptamer

SEQ ID NO: 426 - DNA, 82 bp, Artificial
  clone of aptamer
gggagaggagagaacgttctaccctattgaggttgattggaagtgcctatgtcgctgtcgatcgatcgatcgatgaagggcg

SEQ ID NO: 427 - DNA, 82 bp, Artificial; identical to SEQ ID NO: 426
  clone of aptamer

SEQ ID NO: 428 - DNA, 81 bp, Artificial
  clone of aptamer
gggagaggagagaacgttctactgaagatgttatgatgattgacgaggaggcgctgtcgatcgatcgatcgatgaagggcg

SEQ ID NO: 429 - DNA, 81 bp, Artificial; identical to SEQ ID NO: 428
  clone of aptamer

SEQ ID NO: 430 - DNA, 82 bp, Artificial
  clone of aptamer
gggagaggagagaacgttctactgtctgagtgtcgccgccttgtgtgatgttcgctgtcgatcgatcgatcgatgaagggcg

SEQ ID NO: 431 - DNA, 82 bp, Artificial; identical to SEQ ID NO: 430
  clone of aptamer

SEQ ID NO: 432 - DNA, 82 bp, Artificial
  clone of aptamer
gggagaggagagaacgttctacgtgatggctgtgaatgaggtagttcgaatacgctgtcgatcgatcgatcgatgaagggcg

SEQ ID NO: 433 - DNA, 81 bp, Artificial
  clone of aptamer
gggagaggagagaacgttctacgtgaaatcaaggttgttaatttggggaatcgctgtcgatcgatcgatcgatgaagggcg

SEQ ID NO: 434 - DNA, 82 bp, Artificial
  clone of aptamer
gggagaggagagaacgttctacgtataaggccgtaaccgggtagcgagtggtcgctgtcgatcgatcgatcgatgaagggcg

SEQ ID NO: 435 - DNA, 82 bp, Artificial
  clone of aptamer
gggagaggagagaacgttntacgtgggcgaaggagctgcgggcgttgnagtttgctgtcgatcgatcgatcgatgaagggcg

SEQ ID NO: 436 - DNA, 82 bp, Artificial
  clone of aptamer
gggagaggagagaacgttctacgtcatcctagtctgagatcggattttcttgcgctgtcgatcgatcgatcgatgaagggcg

SEQ ID NO: 437 - DNA, 82 bp, Artificial
  clone of aptamer
gggagaggagagaacgttctacgtttgcgagtgtggtcgacgctgaatgcggcgctgtcgatcgatcgatcgatgaagggcg

SEQ ID NO: 438 - DNA, 82 bp, Artificial
  clone of aptamer
gggagaggagagaacgttctacggattgatagggattgagatgaggtcttgtcgctgtcgatcgatcgatcgatgaagggcg

SEQ ID NO: 439 - DNA, 81 bp, Artificial
  clone of aptamer
gggagaggagagaacgttctacgatgtcgtgttagattacttattgctatctgctgtcgatcgatcgatcgatgaagggcg

SEQ ID NO: 440 - DNA, 82 bp, Artificial
  clone of aptamer
gggagaggagagaacgttctacgatgcctggcggaaacggagcctgggatttcgctgtcnatcgatcgatcgatgaagggcg

SEQ ID NO: 441 - DNA, 80 bp, Artificial
  clone of aptamer
gggagaggagagaacgttctacgaggatttgacgtgtgtgtgctagagtacgctgtcgatcgatcgatcgatgaagggcg

SEQ ID NO: 442 - DNA, 82 bp, Artificial
  clone of aptamer
gggagaggagagaacgttctacgagtattatgcgtcccttgaggatacacggcgctgtcgatcgatcgatcgatgaagggcg

SEQ ID NO: 443 - DNA, 82 bp, Artificial
  clone of aptamer
gggagaggagagaacgttctacagggataactgtagcgatgaaagtaaacgatgctgtcgatcgatcgatcgatgaagggcg

SEQ ID NO: 444 - DNA, 81 bp, Artificial
  clone of aptamer
gggagaggagagaacgttctacaagaagtgtggccgcagagacgaaatgcacgctgtcgatcgatcgatcgatgaagggcg

SEQ ID NO: 445 - DNA, 83 bp, Artificial
  clone of aptamer
gggagaggagagaacgttctacccatatcttccttctttattccgttagttgccgctgtcgatcgatcgatcgatgaagggcg

SEQ ID NO: 446 - DNA, 82 bp, Artificial
  clone of aptamer
gggagaggagagaacgttctacctgtgttgatgcttccgtttgagattgccccgctgtcgatcgatcgatcgatgaagggcg

SEQ ID NO: 447 - DNA, 84 bp, Artificial
  clone of aptamer
gggagaggagagaacgttctaccngtaaganaanctattttagcccttgnnctgcgctgtcgatcgatcgatcgatgaagggcg

SEQ ID NO: 448 - DNA, 83 bp, Artificial
  clone of aptamer
gggagaggagagaacgttctacccttgtcctccaatcctcttttgactcttgccgctgtcgatcgatcgatcgatgaagggcg

SEQ ID NO: 449 - DNA, 82 bp, Artificial
  clone of aptamer
gggagaggagagaacgttctacctgattttgtcactggattccgatggctttcgctgtcgatcgatcgatcgatgaagggcg

SEQ ID NO: 450 - DNA, 83 bp, Artificial
  clone of aptamer
gggagaggagagaacgttctactgtaataagggatgcgtcaggaacctgtgttcgctgtcgatcgatcgatcgatgaagggcg

SEQ ID NO: 451 - DNA, 81 bp, Artificial
  clone of aptamer
gggagaggagagaacgttctactgctttccgggaatttgtttgtttgcttccgctgtcgatcgatcgatcgatgaagggcg

SEQ ID NO: 452 - DNA, 82 bp, Artificial
  clone of aptamer
gggagaggagagaacgttctacttcgtcggttgacttttcttcgtgtagtgtcgctgtcgatcgattgatcgatgaagggcg

SEQ ID NO: 453 - DNA, 92 bp, Artificial
  aptamer library template
catcgatcgatcgatcgacagcgnnnnnnnnnnnnnnnnnnnnnnnnnnnnnngtagaacgttctctcctctccctatagtgagtcgtatta

SEQ ID NO: 454 - DNA, 24 bp, Artificial
  PCR 3′-primer
catcgatgctagtcgtaacgatcc

SEQ ID NO: 455 - DNA, 40 bp, Artificial
  PCR 5′-primer
taatacgactcactatagggagaggagagaaacgttctcg

SEQ ID NO: 456 - RNA, 75 nt, Artificial
  rRmY aptamer ARC256 RNA transcription product
gggagaggagagaacguucuacnnnnnnnnnnnnnnnnnnnnnnnnnnnnnncgcugucgaucgaucgaucgaug

SEQ ID NO: 457 - RNA, 11 nt, Artificial
  mN PEG 5′ oligonucleotide
ggngcngcncc

SEQ ID NO: 458 - RNA, 19 nt, Artificial
  mN PEG 3′ oligonucleotide
ggugccnngucguugcucc

SEQ ID NO: 459 - DNA, 75 bp, Artificial
  aptamer library template
gggagaggagagaacgttctacnnnnnnnnnnnnnnnnnnnnnnnnnnnnnncgctgtcgatcgatcgatcgatg

SEQ ID NO: 460 - DNA, 22 bp, Artificial
  PCR primer
gggagaggagagaacgttctac

SEQ ID NO: 461 - DNA, 22 bp, Artificial
  PCR primer
catcgatcgatcgatcgacagc

SEQ ID NO: 462 - RNA, 75 nt, Artificial
  rGmH aptamer ARC256 transcription product
gggngnggngngnncguucuncnnnnnnnnnnnnnnnnnnnnnnnnnnnnnncgcugucgnucgnucgnucgnug

SEQ ID NO: 463 - RNA, 75 nt, Artificial; identical to SEQ ID NO: 462
  r/mGmH aptamer ARC256 transcription product

SEQ ID NO: 464 - DNA, 75 bp, Artificial
  dRmY aptamer ARC256 transcription product
gggagaggagagaacguucuacnnnnnnnnnnnnnnnnnnnnnnnnnnnnnncgcugucgaucgaucgaucgaug

SEQ ID NO: 465 - DNA, 11 bp, Artificial
  dRmY PEG 5′ oligonucleotide
ggagcagcacc

SEQ ID NO: 466 - DNA, 19 bp, Artificial
  dRmY PEG 3′ oligonucleotide
ggugccaagucguugcucc

SEQ ID NO: 467 - DNA, 80 bp, Artificial
  clone of aptamer
gggagaggagagaacgttctacttgctgtgacggacgggcttgagaggctcgctgtcgatcgatcgatcgatgaagggcg

SEQ ID NO: 468 - RNA, 75 nt, Artificial; identical to SEQ ID NO: 456
  rN aptamer ARC256 transcription product